(19) United States
(12) Patent Application Publication
(10) Pub. No.: US 2017/0369492 A1
(43) Pub. Date: Dec. 28, 2017

(54) 1,3-SUBSTITUTED 2-AMINO-INDOLE DERIVATIVES AND ANALOGUES USEFUL IN THE TREATMENT OR PREVENTION OF DIABETES MELLITUS, OBESITY, AND INFLAMMATORY BOWEL DISEASE

(71) Applicant: Takeda Pharmaceutical Company Limited, Osaka-shi, Osaka (JP)

(72) Inventors: Richard DAVENPORT, Cambridgeshire (GB); Jonathan DUNN, Cambridgeshire (GB); William FARNABY, Cambridgeshire (GB); Duncan HANNAH, Cambridgeshire (GB); David HARRISON, Cambridgeshire (GB); Susanne WRIGHT, Cambridgeshire (GB)

(21) Appl. No.: 15/320,863

(22) PCT Filed: Jun. 24, 2015
(86) PCT No.: PCT/GB2015/051841

§ 371 Date: Dec. 21, 2016

(30) Foreign Application Priority Data
Jun. 25, 2014 (GB) 1411239.5

(51) Int. Cl. C07D 487/04; C07D 471/04; A61K 31/55; A61K 31/5377; A61K 31/437; A61K 31/519; A61K 31/5025; A61K 31/4985; C07D 519/00; A61K 31/541

(52) U.S. Cl. CPC C07D 487/04; C07D 519/00; C07D 471/04; A61K 31/55; A61K 31/541; A61K 31/437; A61K 31/519; A61K 31/5025; A61K 31/4985; A61K 31/5377

(57) ABSTRACT

The present invention provides compounds of formula (I) and pharmaceutically acceptable salts thereof, wherein Q, X⁴, X⁵, X⁶, X⁷, R¹, R², R³ and R⁸ are as defined in the specification, processes for the preparation of such compounds, pharmaceutical compositions containing them and the use of such compounds in therapy.

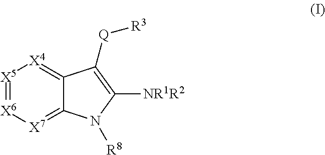

### TECHNICAL FIELD

The present invention relates to 1,3-substituted 2-amino-indole derivatives and analogues, processes for their preparation, pharmaceutical compositions containing them and their use in therapy, particularly in the treatment or prevention of conditions having an association with the GPR43 receptor, such as diabetes mellitus, obesity and inflammatory bowel disease.

### BACKGROUND OF THE INVENTION

Targeting the release of anorectic and antidiabetic gut peptides is the focus of many ongoing drug development programs, as evidence is accumulating that enhanced secretion of Peptide YY (PYY) and Glucagon-Like Peptide-1 (GLP-1) from intestinal L-cells may translate into beneficial effects in subjects with diabetes and obesity.

Short chain fatty acids (SCFA), derived from bacterial fermentation of macrofibrous material reaching the distal gut are known to reach high concentrations under physiological conditions in the colons of healthy subjects. Non-digestible and fermentable dietary fibre, as well as SCFA themselves, have been shown to increase GLP-1 and PYY secretion in humans (Zhou et al., Am. J. Physiol. Endocrinol. Metab., 2008, vol. 295(5), pp. E1160-E1166), and enhanced PYY release has been proposed as a link between luminal SCFA and altered gut motility (Dumoulin et al., Endocrinology, 1998, vol. 139(9), pp. 3780-3786).

SCFA act as a local nutrient source, but can also trigger cell-specific signalling cascades by activation of the G-protein coupled free fatty acid receptors, GPR41 (FFAR³) and GPR43 (FFAR²) (Brown et al., J. Biol. Chem., 2003, vol. 278(13), pp. 11312-11319). The finding that both receptors are located in colonic L cells by immunostaining (Tazoe et al., Biomed. Res., 2009, vol. 30(3), pp. 149-156), suggests that short chain fatty acids may utilise this pathway to modulate L-cell function. In addition to L cells, GPR43 is also expressed in Islets of Langerhans, white adipose tissue, bone marrow and spleen.

GPR43 knockout mice have impaired glucose tolerance, with reduced insulin secretion and reduced GLP-1 secretion (Tolhurst et al., Diabetes, 2012, vol. 61, pp. 364-371). They have increased fat mass and a mild increase in food intake. From this it can be deduced that activation of the GPR43 receptor should lead to beneficial effects in the treatment of diabetes and obesity.

GPR43 is also expressed on a variety of immune cells, so may represent a potential treatment for certain inflammatory diseases and conditions (Bindels L B, Dewulf E M, Delzenne N M., Trends Pharmacol Sci., 2013, 34(4), Pp. 226-32; Macia L et al., Nat Commun, 2015, 6, article 6734; and Smith, P M et al, Science, 2013, 341 (6145), pp. 569-573).

There is therefore a need for compounds that activate the GPR43 receptor.

Certain 3-substituted 2-amino-indole analogues are known in the art. WO 2004/060893 describes a broad class of such compounds useful for treating a variety of diseases modulated by potassium channels. Other substituted indole analogues are known from WO 2012/064897, WO 2005/023818, WO 2011/140164, WO 2011/153553 and US 2014/0018361.

### SUMMARY OF THE INVENTION

In accordance with the present invention, there is provided a compound of formula (I):

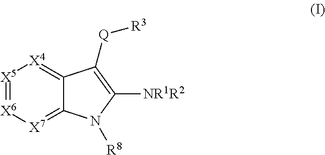

or a pharmaceutically acceptable salt thereof, wherein
Q represents —O—, —S—, —SO—, —SO₂—, —SO₂NR—, —SO₂(CH₂)ₘ— or —SO₂O—;
R represents a hydrogen atom or a C₁-C₆ alkyl group;
m is 1 or 2;
X⁴ represents N or CR⁴;
X⁵ represents N or CR⁵;
X⁶ represents N or CR⁶;
X⁷ represents N or CR⁷;
   provided that one or two of X⁴, X⁵, X⁶ and X⁷ represents a nitrogen atom;
R¹ and R² each independently represent a hydrogen atom or a C₁-C₆ alkyl, C₃-C₈ cycloalkyl or C₁-C₆ alkoxycarbonyl group, each of which may be optionally substituted by at least one halogen atom;
R³ represents a saturated or unsaturated 3- to 10-membered ring system which may comprise at least one ring heteroatom independently selected from nitrogen, oxygen and sulphur, wherein the 3- to 10-membered ring system is optionally substituted by at least one substituent independently selected from halogen, hydroxyl, cyano, C₁-C₆ alkyl, C₁-C₆ haloalkyl, C₁-C₆ hydroxyalkyl, C₁-C₆ alkoxy, C₁-C₆ haloalkoxy, C₃-C₆ cycloalkylC₁-C₆ alkoxy, C₁-C₆ alkoxyC₁-C₆ alkyl, C₁-C₆ alkylC(O)NR¹⁴—, phenyl, (halo)phenylcarbonyl, phenoxy, benzyl, benzyloxycarbonyl and a saturated or unsaturated 4- to 6-membered heterocyclyl group, which heterocyclyl group is itself optionally substituted by at least one C₁-C₆ alkyl group,
and when Q represents —SO₂NR—, R³ may additionally represent a C₁-C₆ alkyl group optionally substituted by at least one substituent independently selected from halogen, C₁-C₆ alkoxy, C₃-C₆ cycloalkyl, phenyl and a saturated or unsaturated 4- to 6-membered heterocyclyl group;
R⁴, R⁵ and R⁶ each independently represent a hydrogen or a halogen atom, or a C₁-C₆ alkyl, C₁-C₆ alkoxy, C₁-C₆ alkylthio, C₁-C₆ haloalkyl, NR¹²R¹³, C₃-C₈ cycloalkyl or C₅-C₈ cycloalkenyl group;
R⁷ represents a hydrogen or a halogen atom, hydroxyl, cyano, NR⁹R¹⁰, or a C₁-C₆ alkyl, C₃-C₈ cycloalkyl, C₂-C₆ alkenyl, C₅-C₈ cycloalkenyl, C₁-C₆ alkoxy, C₃-C₈ cycloalkyloxy, benzyloxy, 3- to 11-membered saturated heterocyclyl, 3- to 11-membered saturated heterocyclyloxy, C₆-C₁₀ aryl or heteroaryl group, each of which may be optionally substituted by at least one substituent independently selected from halogen, cyano, C₁-C₆ alkyl, C₁-C₆ alkoxy, C₃-C₈ cycloalkyl, phenyl and a saturated or unsaturated 4- to 6-membered heterocyclyl group wherein each C₁-C₆ alkyl, C₁-C₆ alkoxy, C₃-C₈ cycloalkyl, phenyl or saturated or unsaturated 4- to 6-membered heterocyclyl substituent group may itself be optionally substituted by at least one substituent independently selected from halogen, C₁-C₃ alkyl, C₁-C₃ alkoxy and C₃-C₆ cycloalkyl;
either R⁸ represents a saturated 3- to 8-membered ring system which may comprise at least one ring heteroatom independently selected from nitrogen, oxygen and sulphur, wherein the 3- to 8-membered ring system is optionally substituted by at least one substituent independently selected from halogen, hydroxyl and C₁-C₆ alkyl, or R⁸ represents a C₁-C₆ alkyl group optionally substituted by at least one substituent independently selected from phenyl and C₃-C₆ cycloalkyl, the cycloalkyl group itself being optionally substituted by at least one C₁-C₆ alkyl group;
R⁹ and R¹⁰ each independently represent a hydrogen atom, or a C₁-C₆ alkyl or —(CH₂)ₚ—R¹¹ group, each of which may be optionally substituted by at least one substituent independently selected from halogen, C₁-C₃ alkyl and C₁-C₃ alkoxy;
p is 0 or 1;
R¹¹ represents C₃-C₆ cycloalkyl, phenyl or a saturated or unsaturated 5- to 6-membered heterocyclyl group; and
R¹², R¹³ and R¹⁴ each independently represent a hydrogen atom or a C₁-C₆ alkyl group.

In the context of the present specification, unless otherwise stated, an “alkyl” substituent group or an alkyl moiety in a substituent group may be linear or branched. Examples of alkyl groups/moieties include methyl, ethyl, n-propyl, i-propyl, n-butyl, i-butyl, t-butyl, n-pentyl, and n-hexyl.

A “haloalkyl” substituent group or a haloalkyl moiety in a substituent group refers to an alkyl group or moiety in which one or more, e.g. one, two, three, four or five, hydrogen atoms are replaced independently by halogen atoms, i.e. by fluorine, chlorine, bromine or iodine atoms. Examples of haloalkyl groups/moieties include fluoromethyl, difluoromethyl, trifluoromethyl, 2,2-difluoroethyl and 2,2,2-trifluoroethyl.

A “hydroxyalkyl” substituent group or a hydroxyalkyl moiety in a substituent group refers to an alkyl group or moiety in which one or more, e.g. one, two, three, four or five, hydrogen atoms are replaced by hydroxyl groups, examples of which include —CH₂OH, —CH₂CH₂OH, —CH₂CH₂CH₂OH, —CH(OH)CH₂OH, —CH(CH₃)OH and —CH(CH₂OH)₂.

The term “(halo)phenylcarbonyl” denotes a phenylcarbonyl group which is optionally substituted with from 1 to 5 independently selected halogen atoms, an example of which is fluorophenylcarbonyl.

A “cycloalkyl” substituent group or a cycloalkyl moiety in a substituent group refers to a saturated hydrocarbyl ring containing, for example, from 3 to 8 carbon atoms, examples of which include cyclopropyl, cyclobutyl, cyclopentyl and cyclohexyl. Unless stated otherwise, a cycloalkyl substituent group or moiety may include monocyclic, bicyclic (e.g. fused or spiro) and polycyclic hydrocarbyl rings.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl.

A “cycloalkenyl” substituent group or a cycloalkenyl moiety in a substituent group refers to an unsaturated hydrocarbyl ring having one or more carbon-carbon double bonds and containing, for example, from 3 to 8 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic (e.g. fused or spiro) and polycyclic hydrocarbyl rings.

A “C₆-C₁₀ aryl” group refers to a group derived from an aromatic hydrocarbon containing from six to ten carbon atoms. The aryl group may be monocyclic or polycyclic (e.g. bicyclic) in which the two or more rings are fused, examples of which include phenyl, 1-naphthyl and 2-naphthyl. Also included within the scope of the term “aryl”, as it is used herein, is a group in which an aromatic ring is fused to one or more non-aromatic rings as exemplified by indanyl and tetrahydronaphthyl. An aryl group may be bonded at any suitable ring atom.

A “heteroaryl” group is a 5- to 10-membered aryl group in which from 1 to 4 ring carbon atoms are replaced by heteroatoms independently selected from nitrogen, oxygen and sulphur. The heteroaryl group can be bonded at any suitable ring atom (i.e. at any carbon or heteroatom of the heteroaryl ring system). Examples of heteroaryl groups include the following:

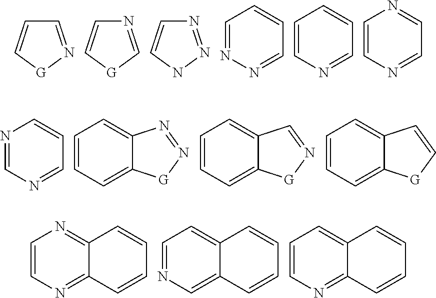

G=O, S or NH

The term “halogen” includes fluorine, chlorine, bromine and iodine.

When a group or moiety is described as being ‘unsaturated’, it should be understood that the group or moiety may be partially or fully unsaturated and thus may have aliphatic or aromatic properties.

For the purposes of the present invention, where a combination of moieties is referred to as one group, for example, arylalky or alkoxycarbonyl, the last mentioned moiety contains the atom by which the group is attached to the rest of the molecule. An example of an arylalkyl group is benzyl and an example of an alkoxycarbonyl group is —C(O)OCH₃.

It will be appreciated that the invention does not encompass any unstable structures or any divalent —O—O—, —O—S— or —S—S— moieties. When any chemical moiety or group is described as being optionally substituted, it will be appreciated that the moiety or group may be either unsubstituted or substituted by one or more of the specified substituents. It will be appreciated that the number and nature of substituents will be selected so as to avoid sterically undesirable combinations.

In an embodiment of the invention, one of X⁴, X⁵, X⁶ and X⁷ is N, e.g. X⁴ is N or X⁷ is N.

In another embodiment of the invention, two of X⁴, X⁵, X⁶ and X⁷ are N, e.g.

X⁴ and X⁷ are N, X⁵ is CR⁵ and X⁶ is CR⁶, or
X⁵ and X⁷ are N, X⁴ is CR⁴ and X⁶ is CR⁶, or
X⁴ and X⁶ are N, X⁵ is CR⁵ and X⁷ is CR⁷, or
X⁶ and X⁷ are N, X⁴ is CR⁴ and X⁵ is CR⁵.

In a particular embodiment, X⁴ and X⁷ are N, X⁵ is CR⁵ and X⁶ is CR⁶.

As stated above, Q represents —O—, —S—, —SO—, —SO₂—, —SO₂NR—, —SO₂(CH₂)ₘ— or —SO₂O—. When Q represents an SO₂NR—, —SO₂(CH₂)ₘ— or —SO₂O— group, the group will be attached to the central ring system through the sulphur atom.

In one embodiment of the invention, Q represents —SO₂— or —SO₂NR—.

R represents a hydrogen atom or a C₁-C₆, or C₁-C₄, or C₁-C₂ alkyl group. In one embodiment, R represents a hydrogen atom or a methyl group.

In a further embodiment, Q represents —SO₂—.

As stated above, R¹ and R² each independently represent a hydrogen atom or a C₁-C₆, or C₁-C₄, or C₁-C₂ alkyl, C₃-, C₄-, C₅- or C₆-C₈ cycloalkyl or C₁-C₆, or C₁-C₄, or C₁-C₂ alkoxycarbonyl group, each of which may be optionally substituted by at least one halogen atom, e.g. one, two, three or four halogen atoms independently selected from fluorine and chlorine atoms.

In one embodiment, R¹ and R² each independently represent a hydrogen atom or a C₁-C₆, or C₁-C₄, or C₁-C₂ alkyl, C₃-C₆ cycloalkyl or C₁-C₆, or C₁-C₄, or C₁-C₂ alkoxycarbonyl group, each of which may be optionally substituted by one or two halogen atoms independently selected from fluorine and chlorine atoms.

In another embodiment, R¹ and R² each independently represent a hydrogen atom.

In a further embodiment, one of R¹ and R² represents a hydrogen atom and the other of R¹ and R² represents a C₁-C₂ alkyl (such as methyl), C₃-C₆ cycloalkyl (such as cyclohexyl) or C₁-C₂ alkoxycarbonyl (such as methoxycarbonyl) group, each of which may be optionally substituted by one or two fluorine atoms.

Examples of R¹ and R² substituents include hydrogen atoms and methyl, 4,4-difluorocyclohexyl and methoxycarbonyl groups.

As stated above, R⁵ represents a saturated or unsaturated 3- to 10-membered (e.g. 3-, 4-, 5- or 6- to 7-, 8-, 9- or 10-membered) ring system which may comprise at least one ring heteroatom (e.g. one, two, three or four ring heteroatoms) independently selected from nitrogen, oxygen and sulphur, wherein the 3- to 10-membered ring system is optionally substituted by at least one substituent (e.g. one, two, three or four substituents) independently selected from halogen (e.g. fluorine, chlorine, bromine or iodine), hydroxyl, cyano, C₁-C₆, or C₁-C₄, or C₁-C₂, alkyl, C₁-C₆, or C₁-C₄, or C₁-C₂ haloalkyl, C₁-C₆, or C₁-C₄, or C₁-C₂ hydroxyalkyl, C₁-C₆, or C₁-C₄, or C₁-C₂ alkoxy, C₁-C₆, or C₁-C₄, or C₁-C₂ haloalkoxy, C₃-C₆ cycloalkylC₁-C₆ alkoxy (e.g. cyclopropylC₁-C₆, or C₁-C₄, or C₁-C₂ alkoxy, specifically cyclopropylmethoxy), C₁-C₆ alkoxyC₁-C₆ alkyl (e.g. C₁-C₆, or C₁-C₄, or C₁-C₂ alkoxymethyl, specifically methoxymethyl), C₁-C₆, or C₁-C₄, or C₁-C₂ alkylC(O)NR¹⁴, phenyl, (halo)phenylcarbonyl, phenoxy, benzyl, benzyloxycarbonyl and a saturated or unsaturated 4- to 6-membered heterocyclyl group, which heterocyclyl group is itself optionally substituted by at least one C₁-C₆, or C₁-C₄, or C₁-C₂ alkyl group,

and when Q represents —SO₂NR—, R³ may additionally represent a C₁-C₆, or C₁-C₄, or C₁-C₂ alkyl group optionally substituted by at least one substituent (e.g. one, two, three or four substituents) independently selected from halogen (e.g. fluorine, chlorine, bromine or iodine), C₁-C₆, or C₁-C₄, or C₁-C₂ alkoxy, C₁-C₆ cycloalkyl, phenyl and a saturated or unsaturated 4- to 6-membered heterocyclyl group.

This R³ saturated or unsaturated 3- to 10-membered ring system may comprise one or more (e.g. one, two, three or four) ring heteroatoms independently selected from nitrogen, oxygen and sulphur. The ring system may be monocyclic or polycyclic (e.g. bicyclic) in which the two or more rings are fused, bridged or spiro. If the ring system is unsaturated, it may be partially or fully unsaturated. The ring system can be bonded to Q at any suitable ring atom (i.e. at any carbon or heteroatom of the ring system).

Examples of R⁵ saturated or unsaturated 3- to 10-membered ring systems include cyclopropyl, cyclobutyl, cyclopentyl, cyclohexyl, cyclopentenyl, cyclohexenyl, bicyclo[2.2.1]heptyl, azabicyclo[3.2.1]octanyl, phenyl, azetidinyl, pyrrolidinyl, piperidinyl, piperazinyl, morpholinyl, thiomorpholinyl, oxadiazolyl (e.g. 1,2,4-oxadiazolyl), tetrahydrofuranyl, naphthyl, benzofuranyl, benzothienyl, benzodioxolyl, 2,3-dihydro-1,4-benzodioxinyl, benzoxazolyl, quinolinyl, isoquinolinyl, 1,2,3,4-tetrahydroquinolinyl, 1,2,3,4-tetrahydroisoquinolinyl, oxazolyl, thiadiazolyl (e.g. 1,2,3-thiadiazolyl), 2,3-dihydroindenyl, 1,4-oxazepanyl, azepanyl, 2,3-dihydrobenzofuranyl, 2,3-dihydroisoindolyl, tetrahydropyranyl, 2,3-dihydro-1H-pyrrolo[3,4-c]pyridinyl, pyrazolyl, imidazo[1,2-a]pyridinyl, pyrazinyl, thiazolidinyl, indanyl, thienyl, isoxazolyl, pyridazinyl, pyrrolyl, furanyl, thiazolyl, isothiazolyl, indolyl, isoindolyl, imidazolyl, pyrimidinyl, benzimidazolyl, triazolyl, tetrazolyl and pyridinyl.

In one aspect, the R³ saturated or unsaturated 3- to 10-membered ring system is selected from phenyl, thienyl, cyclopropyl, cyclohexyl, pyridinyl, pyrrolidinyl, piperidinyl, piperazinyl, morpholinyl, azetidinyl, 1,4-oxazepanyl, azepanyl, thiomorpholinyl, 1,2,3,4-tetrahydroisoquinolinyl, 2,3-dihydroisoindolyl, azabicyclo[3.2.1]octanyl and 2,3-dihydro-1,4-benzodioxinyl.

If present in R³, a saturated or unsaturated 4- to 6-membered heterocyclyl substituent group contains from 1 to 4 ring heteroatoms independently selected from nitrogen, oxygen and sulphur, examples of which include azetidinyl, oxetanyl, pyrrolidinyl, piperidinyl, morpholinyl, thiomorpholinyl, piperazinyl, tetrahydrofuranyl, tetrahydropyranyl, oxadiazolyl, pyrrolyl, imidazolyl, pyridinyl, pyrazinyl, pyridazinyl, pyrimidinyl, thienyl and furanyl.

In one embodiment of the invention, R⁵ represents a saturated or 3-, 4-, 5- or 6-membered ring system which may comprise at least one ring heteroatom (e.g. one, two, three or four ring heteroatoms) independently selected from nitrogen, oxygen and sulphur, wherein the 3-, 4-, 5- or 6-membered ring system is optionally substituted by at least one substituent (e.g. one, two, three or four substituents) independently selected from halogen (e.g. fluorine, chlorine, bromine or iodine), hydroxyl, cyano, C₁-C₂ alkyl, C₁-C₂ haloalkyl, C₁-C₂ hydroxyalkyl, C₁-C₂ alkoxy, C₁-C₂ haloalkoxy, C₃-C₆ cycloalkylC₁-C₂ alkoxy, C₁-C₂ alkoxyC₁-C₂ alkyl, C₁-C₂ alkylC(O)NR¹⁴—, phenyl, (halo)phenylcarbonyl, phenoxy, benzyl, benzyloxycarbonyl and a saturated or unsaturated 4- to 6-membered heterocyclyl group, which heterocyclyl group is itself optionally substituted by at least one, e.g. one or two, C₁-C₆, or C₁-C₄, or C₁-C₂ alkyl groups which may be the same or different to one another,

and when Q represents —SO₂NR—, R³ may additionally represent a C₁-C₄ alkyl group optionally substituted by at least one substituent (e.g. one, two, three or four substituents) independently selected from halogen (e.g. fluorine, chlorine, bromine or iodine), C₁-C₂ alkoxy, C₃-C₆ cycloalkyl, phenyl and a saturated or unsaturated 4- to 6-membered heterocyclyl group.

In another embodiment, R³ represents a saturated 4- to 6-membered ring system which may comprise one or two ring heteroatoms independently selected from nitrogen, oxygen and sulphur (e.g. cyclohexyl, azetidinyl, pyrrolidinyl, piperidinyl, piperazinyl, thiomorpholinyl or morpholinyl), wherein the saturated 4- to 6-membered ring system is optionally substituted by at least one substituent (e.g. one, two, three or four substituents) independently selected from halogen (e.g. fluorine, chlorine, bromine or iodine), hydroxyl, C₁-C₂ alkyl, C₁-C₂ haloalkyl, C₁-C₂ hydroxyalkyl, C₁-C₂ alkoxy, C₁-C₂ haloalkoxy, C₃-C₆ cycloalkylC₁-C₂ alkoxy, C₁-C₂ alkoxyC₁-C₂ alkyl, C₁-C₂ alkylC(O)NR¹⁴—, phenyl, fluorophenylcarbonyl, phenoxy, benzyl and a saturated or unsaturated 4- to 6-membered heterocyclyl group, which heterocyclyl group is itself optionally substituted by at least one C₁-C₂ alkyl group.

In an alternative embodiment, R³ represents an unsaturated, e.g. aromatic, 6- to 10-membered ring system which may comprise at least one ring heteroatom (e.g. one, two, three or four ring heteroatoms) independently selected from nitrogen, oxygen and sulphur, wherein the unsaturated 6- to 10-membered ring system is optionally substituted by at least one substituent (e.g. one, two, three or four substituents) independently selected from halogen (e.g. fluorine, chlorine, bromine or iodine), cyano, C₁-C₆, or C₁-C₄, or C₁-C₂ alkyl, C₁-C₆, or C₁-C₄, or C₁-C₂ haloalkyl, C₁-C₆, or C₁-C₄, or C₁-C₂ alkoxy, C₁-C₆, or C₁-C₄, or C₁-C₂ haloalkoxy, benzyloxycarbonyl and a saturated or unsaturated 5- to 6-membered heterocyclyl group, which heterocyclyl group is itself optionally substituted by at least one, e.g. one or two, C₁-C₆, or C₁-C₄, or C₁-C₂ alkyl groups which may be the same or different to one another.

In a further embodiment, R³ represents a phenyl or pyridinyl group which is optionally substituted by at least one substituent (e.g. one, two, three or four substituents) independently selected from halogen (e.g. fluorine or chlorine), cyano, C₁-C₂ alkyl, C₁-C₂ haloalkyl (e.g. trifluoromethyl), C₁-C₄ alkoxy, C₁-C₂ haloalkoxy (e.g. difluoromethoxy or trifluoromethoxy), benzyloxycarbonyl and a saturated or unsaturated 5- to 6-membered heterocyclyl group (e.g. morpholinyl), which heterocyclyl group is itself optionally substituted by at least one, e.g. one or two, C₁-C₆, or C₁-C₄, or C₁-C₂ alkyl groups which may be the same or different to one another.

In a still further embodiment, R⁵ represents phenyl optionally substituted by one or two substituents independently selected from fluorine, chlorine, cyano, methyl, trifluoromethyl, difluoromethoxy, trifluoromethoxy and C₁-C₃ alkoxy.

In yet another embodiment, R³ represents an unsubstituted phenyl group.

In still another embodiment, when Q represents —SO₂NR—, R³ represents a C₁-C₄ alkyl group optionally substituted by at least one substituent (e.g. one, two, three or four substituents) independently selected from halogen (e.g. fluorine, chlorine, bromine or iodine), C₁-C₂ alkoxy, C₃-C₆ cycloalkyl, phenyl and a saturated or unsaturated 4- to 6-membered heterocyclyl group (e.g. oxetanyl, tetrahydrofuranyl or thiazolyl).

In a particular embodiment of the invention, R³ represents any one of the following moieties or is selected from a group containing any two or more of such moieties:
(i) 1-N-benzylcarboxylate-piperidin-4-yl,
(ii) 2,3-difluorophenyl,
(iii) 2-fluoro-4-methoxyphenyl,
(iv) 2-fluoro-4-methylphenyl,
(v) 2-fluorophenyl,
(vi) 2-methoxyphenyl,
(vii) 2-methylphenyl,
(viii) 3,4-difluorophenyl,
(ix) 3,5-difluorophenyl,
(x) 3-chloro-4-methoxyphenyl,
(xi) 3-fluoro-4-methoxyphenyl,
(xii) 3-fluorophenyl,
(xiii) 3-methoxyphenyl,
(xiv) 3-methylphenyl,
(xv) 4-(difluoromethoxy)phenyl,
(xvi) 4-(fluoromethoxy)phenyl,
(xvii) 4-(propan-2-yloxy)phenyl,
(xviii) 4-(trifluoromethyl)phenyl,
(xix) 4-bromo-2-[(2S)-2-methylmorpholin-4-yl]-phenyl,
(xx) 4-bromo-2-fluorophenyl,
(xxi) 4-chloro-2-fluorophenyl,
(xii) 4-chloro-3-fluorophenyl,
(xxiii) 4-chlorophenyl,
(xxiv) 4-fluoro-2-methoxyphenyl,
(xxv) 4-fluoro-2-methylphenyl,
(xxvi) 4-fluorophenyl,
(xxvii) 4-methoxyphenyl,
(xxviii) 4-methylphenyl,
(xxix) 4-cyanophenyl,
(xxx) 6-methoxypyridin-3-yl,
(xxxi) tetrahydrofuranylmethyl,
(xxxii) 2-methoxyethyl,
(xxxiii) (1,3-thiazol-2-yl)ethyl,
(xxxiv) propyl,
(xxxv) 3,3,3-trifluoropropyl,
(xxxvi) butyl,
(xxxvii) cyclopropyl,
(xxxviii) cyclopropylmethyl,
(xxxix) cyclobutylmethyl,
(xl) cyclohexyl,
(xli) oxan-4-yl,
(xlii) oxolan-3-yl,
(xliii) phenyl,
(xliv) 2-phenylethyl,
(xlv) pyridin-2-yl,
(xlvi) pyridin-3-yl,
(xlvii) benzyl,
(xlviii) thienyl,
(xlix) azetidinyl,
(l) 3-methoxyazetidin-1-yl,
(li) 3-phenoxyazetidin-1-yl,
(lii) 3-(piperidin-1-yl)azetidin-1-yl,
(liii) 3-(pyrazol-1-yl)azetidin-1-yl,
(liv) pyrrolidinyl,
(lv) 2-methylpyrrolidin-1-yl,
(lvi) 3-methylpyrrolidin-1-yl,
(lvii) 3,3-dimethylpyrrolidin-1-yl,
(lviii) 3-methoxypyrrolidin-1-yl,
(lix) 3-(methoxymethyl)pyrrolidin-1-yl,
(lx) 3-phenylpyrrolidin-1-yl,
(lxi) piperidinyl,
(lxii) 4-hydroxypiperidin-1-yl,
(lxiii) 4-hydroxymethylpiperidin-1-yl,
(lxiv) 3-methylpiperidin-1-yl,
(lxv) 4-methylpiperidin-1-yl,
(lxvi) 3,3-dimethylpiperidin-1-yl,
(lxvii) 4,4-dimethylpiperidin-1-yl,
(lxviii) 4-methoxypiperidin-1-yl,
(lxix) 4-ethoxypiperidin-1-yl,
(lxx) 4,4-difluoropiperidin-1-yl,
(lxxi) 4-(trifluoromethyl)piperidin-1-yl,
(lxxii) 4-(cyclopropylmethoxy)piperidin-1-yl,
(lxxiii) 4-phenylpiperidin-1-yl,
(lxxiv) 4-phenoxypiperidin-1-yl,
(lxxv) 4-benzylpiperidin-1-yl,
(lxxvi) piperazinyl,
(lxxvii) 4-methylpiperazin-1-yl,
(lxxviii) (4-fluorophenylcarbonyl)piperazin-1-yl,
(lxxix) 2,2,2-trifluoroethylpiperazinyl,
(lxxx) morpholinyl,
(lxxxi) 2,6-dimethylmorpholin-4-yl,
(lxxxii) thiomorpholinyl,
(lxxxiii) 1,4-oxazepanyl,
(lxxiv) azepanyl,
(lxxxv) 4-(methylacetamido)piperidin-1-yl,
(lxxxvi) oxetanyl,
(lxxxvii) oxetan-3-ylmethyl,
(lxxxviii) tetrahydroiosquinolinyl,
(lxxxix) 2,3-dihydroisoindol-2-yl,
(xc) azabicyclo[3.2.1]octanyl,
(xci) (hydroxy)azabicyclo[3.2.1]octanyl, and
(xcii) 2,3-dihydro-1,4-benzodioxin-6-yl.

If present, R⁴, R⁵ and R⁶ each independently represent a hydrogen or a halogen atom, or a C₁-C₆, or C₁-C₄, or C₁-C₂ alkyl (e.g. methyl or ethyl), C₁-C₆, or C₁-C₄, or C₁-C₂ alkoxy (e.g. methoxy), C₁-C₆, or C₁-C₄, or C₁-C₂ alkylthio (e.g. methylthio), C₁-C₆, or C₁-C₄, or C₁-C₂ haloalkyl (e.g. trifluoromethyl), NR¹²R¹³ (e.g. dimethylamino), C₃-C₈ cycloalkyl (e.g. cyclopropyl or cyclohexyl) or C₅-C₈ cycloalkenyl (e.g. cyclohexenyl) group.

In an embodiment of the invention, R⁴ represents a hydrogen atom.

In an embodiment of the invention, R⁵ represents a hydrogen or halogen (e.g. chlorine) atom, or a C₁-C₆, or C₁-C₄, or C₁-C₂ alkyl (e.g. methyl or ethyl) group.

In an embodiment of the invention, R⁶ represents a hydrogen atom, or a C₁-C₆, or C₁-C₄, or C₁-C₂ alkyl (e.g. methyl or ethyl) group.

In a further embodiment, R⁵ and R⁶ each independently represent a hydrogen or chlorine atom or a methyl group.

As stated above, R⁷ represents a hydrogen or a halogen atom, hydroxyl, cyano, NR⁹R¹⁰, or a C₁-C₆, or C₁-C₄, or C₁-C₂ alkyl, C₃-, C₄- or C₅- to C₆-, C₇- or C₈-cycloalkyl, C₂-C₆ or C₂-C₄ alkenyl, C₅-C₈ or C₅-C₆ cycloalkenyl, C₁-C₆, or C₁-C₄, or C₁-C₂ alkoxy, C₃-, C₄- or C₅- to C₆-, C₇- or C₈-cycloalkyloxy, benzyloxy, 3- to 11-membered saturated heterocyclyl, 3- to 11-membered saturated heterocyclyloxy, C₆-C₁₀ aryl or heteroaryl group, each of which may be optionally substituted by at least one substituent (e.g. one, two, three or four substituents) independently selected from halogen, cyano, C₁-C₆, or C₁-C₄, or C₁-C₂ alkyl, C₁-C₆, or C₁-C₄, or C₁-C₂ alkoxy, C₃-C₈ or C₃-C₆ cycloalkyl, phenyl and a saturated or unsaturated 5- to 6-membered heterocyclyl group wherein each C₁-C₆ alkyl, C₁-C₆ alkoxy, C₃-C₈ cycloalkyl, phenyl or saturated or unsaturated 5- to 6-membered heterocyclyl substituent group may itself be optionally substituted by at least one substituent (e.g. one, two, three or four substituents) independently selected from halogen, C₁-C₃ alkyl, C₁-C₃ alkoxy and C₁-C₆ cycloalkyl.

The R⁷ 3- to 1-membered saturated heterocyclyl group or moiety contains from 1 to 4 ring heteroatoms independently selected from nitrogen, oxygen and sulphur. Furthermore, the group or moiety may be monocyclic or polycyclic (e.g. bicyclic) in which the two or more rings are fused, bridged or spiro. The R⁷ saturated heterocyclyl group can be bonded to the central ring system through any suitable ring atom (i.e. through any carbon or heteroatom of the heterocyclyl group). Examples of such 3- to 11-membered saturated heterocyclyl groups or moieties include azetidinyl, pyrrolidinyl, piperidinyl, morpholinyl, thiomorpholinyl, azepanyl, oxazepanyl, tetrahydrofuranyl, tetrahydropyranyl, 6-azaspiro[2.5]octanyl, 6-oxa-9-azaspiro[4.5]decanyl, 2-oxa-6-azaspiro[3.5]nonanyl, 4-oxa-7-azaspiro[2.5]octanyl, 5-oxa-8-azaspiro[3.5]nonanyl, 8-oxa-3-azabicyclo[3.2.1]octanyl and octahydrocyclopenta[b]morpholinyl.

The R₇ heteroaryl group contains from 1 to 4 ring heteroatoms independently selected from nitrogen, oxygen and sulphur. The group may be monocyclic, or bicyclic in which the rings are fused together. Specific examples of R₇ heteroaryl groups include pyrrolyl, imidazolyl, pyrazolyl, triazolyl, tetrazolyl, pyridinyl, pyrazinyl, pyrimidinyl, pyridazinyl, triazinyl, thienyl, furyl, furazanyl, oxazolyl, thiazolyl, oxadiazolyl, isothiazolyl, isoxazolyl, thiadiazolyl, tetrazinyl, quinoxalinyl, benzothiazolyl, benzoxazolyl, quinolinyl, quinazolinyl, indolyl, 7-azaindolyl, indolizinyl, indazolyl, imidazo[1,2-a]pyridinyl and 7H-pyrrolo[2,3-d]pyrimidinyl. If present, the R⁷ saturated or unsaturated 5- to 6-membered heterocyclyl substituent group contains from 1 to 4 ring heteroatoms independently selected from nitrogen, oxygen and sulphur, examples of which include pyrrolidinyl, piperidinyl, morpholinyl, piperazinyl, tetrahydrofuranyl, tetrahydropyranyl, dioxolanyl, oxadiazolyl, pyrrolyl, imidazolyl, pyridinyl, pyrazinyl, pyridazinyl, pyrimidinyl, thienyl and furanyl.

In an embodiment of the invention, R⁷ represents a hydrogen or a halogen atom (e.g. fluorine, chlorine or bromine), hydroxyl, cyano, NR⁹R¹⁰, or a C₁-C₄ alkyl, C₃-C₆ cycloalkyl, C₂-C₄ alkenyl, C₅-C₆ cycloalkenyl, C₁-C₆ alkoxy, C₃-C₆ cycloalkyloxy, benzyloxy, 3- to 11-membered saturated heterocyclyl, 3- to 6-membered saturated heterocyclyloxy, C₆-C₁₀ aryl or 5- to 6-membered heteroaryl group, each of which may be optionally substituted by at least one substituent (e.g. one, two, three or four substituents) independently selected from halogen, cyano, C₁-C₄ alkyl, C₁-C₄ alkoxy, C₃-C₆ cycloalkyl, phenyl and a saturated or unsaturated 5- to 6-membered heterocyclyl group wherein each C₁-C₄ alkyl, C₁-C₄ alkoxy, C₃-C₆ cycloalkyl, phenyl or saturated or unsaturated 5- to 6-membered heterocyclyl substituent group may itself be optionally substituted by at least one substituent (e.g. one, two, three or four substituents) independently selected from halogen (e.g. fluorine or chlorine), C₁-C₃ alkyl (e.g. methyl), C₁-C₃ alkoxy (e.g. methoxy) and C₃-C₆ cycloalkyl (e.g. cyclopropyl).

In a second embodiment, R⁷ represents a hydrogen or a halogen atom (e.g. fluorine, chlorine or bromine), hydroxyl, cyano, NR⁹R¹⁰, or a C₁-C₄ alkyl, C₃-C₆ cycloalkyl, C₂-C₄ alkenyl, C₅-C₆ cycloalkenyl, C₁-C₆ alkoxy, C₃-C₆ cycloalkyloxy, benzyloxy, 3- to 6-membered saturated heterocyclyl (e.g. azetidinyl, pyrrolidinyl, piperidinyl, morpholinyl or thiomorpholinyl), 5- to 6-membered saturated heterocyclyloxy (e.g. tetrahydrofuranyloxy or tetrahydropyranyloxy), phenyl, pyrazolyl or pyridinyl group, each of which may be optionally substituted by at least one substituent (e.g. one, two, three or four substituents) independently selected from halogen, cyano, C₁-C₄ alkyl, C₁-C₄ alkoxy, C₃-C₆ cycloalkyl, phenyl and a saturated or unsaturated 5- to 6-membered heterocyclyl group (e.g. tetrahydrofuranyl, tetrahydropyranyl, pyridinyl, pyrazolyl, thiazolyl and oxazolyl), wherein each C₁-C₄ alkyl, C₁-C₄ alkoxy, C₃-C₆ cycloalkyl, phenyl or saturated or unsaturated 5- to 6-membered heterocyclyl substituent group may itself be optionally substituted by at least one substituent (e.g. one, two, three or four substituents) independently selected from halogen (e.g. fluorine or chlorine), C₁-C₃ alkyl (e.g. methyl), C₂-C₃ alkoxy (e.g. methoxy) and C₃-C₆ cycloalkyl (e.g. cyclopropyl).

If R⁷ represents a group NR⁹R¹⁰, then as stated above R⁹ and R¹⁰ each independently represent a hydrogen atom, or a C₁-C₆, or C₁-C₄, or C₁-C₂ alkyl or —(CH₂)ₚ—R¹¹ group, each of which may be optionally substituted by at least one substituent (e.g. one, two, three or four substituents) independently selected from halogen (e.g. fluorine or chlorine), C₁-C₃ alkyl (e.g. methyl) and C₁-C₃ alkoxy (e.g. methoxy).

As stated above, p is 0 or 1 and R¹¹ represents C₃-C₆ cycloalkyl, phenyl or a saturated or unsaturated 5- to 6-membered heterocyclyl group. This R¹¹ saturated or unsaturated 5-to 6-membered heterocyclyl group is as defined above for R⁷.

In one aspect, R⁹ and R¹⁰ each independently represent a hydrogen atom, or a C₁-C₄ alkyl or R¹¹ group, each of which may be optionally substituted as previously defined.

In another aspect, R⁹ and R¹⁰ each independently represent a hydrogen atom, or a C₁-C₄ alkyl or R¹¹ group selected from cyclopropyl, tetrahydrofuranyl and tetrahydropyranyl, each of which may be optionally substituted by at least one substituent (e.g. one, two, three or four substituents) independently selected from fluorine and methyl.

In yet another aspect, one of R⁹ and R¹⁰ represents a hydrogen atom or a C₁-C₆ alkyl (e.g. methyl) group and the other of R⁹ and R¹⁰ represents a group —(CH₂)—R¹¹, each of which may be optionally substituted as previously defined.

In still another aspect, one of R⁹ and R¹⁰ represents a hydrogen atom or a methyl group, and the other of R⁹ and R¹⁰ represents a —(CH₂)—R¹¹ group optionally substituted as previously defined, wherein R¹¹ is selected from oxazolyl, pyridinyl, dioxolanyl, phenyl, tetrahydrofuranyl, tetrahydropyranyl, cyclohexyl, furanyl, cyclopropyl and pyrazolyl.

In a third embodiment, R⁷ is represented by a group of formula:

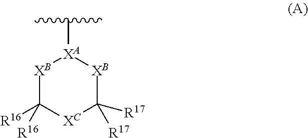

wherein
X^{A} represents N or CH;
each X^{B} independently represents a single bond or —C(R¹⁴)₂—, provided that at least one X^{B} represents —C(R¹⁴)₂—;
each R¹⁴ independently represents a hydrogen or a halogen atom or a cyano, C₁-C₄ alkyl, C₁-C₄ haloalkyl or phenyl group;
X^{C} represents —O—, —S—, —C(R¹⁵)₂— or —NR¹⁵—;
each R¹⁵ independently represents a hydrogen or a halogen atom or a C₁-C₄ alkyl or C₁-C₄ haloalkyl group, or two R¹⁵ groups may together represent a —(C(R¹⁸)₂)ₙ— group, wherein each R¹⁸ independently represents a hydrogen or a halogen atom and n is 2, 3, 4 or 5;
each R¹⁶ independently represents a hydrogen or a halogen atom or a cyano, C₁-C₄ alkyl, C₁-C₄ haloalkyl or phenyl group, or two R¹⁶ may together represent a —(C(R¹⁹)₂)_{q}— group, wherein each R¹⁶ independently represents a hydrogen or a halogen atom and q is 2, 3, 4 or 5; and
each R¹⁷ independently represents a hydrogen or a halogen atom or a cyano, C₁-C₄ alkyl, C₁-C₄ haloalkyl or phenyl group, or two R¹⁷ may together represent a —(C(R²⁰)₂)ₜ— group, wherein each R²⁰ independently represents a hydrogen or a halogen atom and t is 2, 3, 4 or 5.

In one embodiment, X^{A} in formula (A) represents N.

In another embodiment, both X^{B} moieties in formula (A) represent CH₂.

In a further embodiment, in formula (A), one XP represents CH₂ and the other X^{B} represents CH(CH₃), or one X^{B} represents CH₂ and the other X^{B} represents a single bond.

In one embodiment, X^{C} in formula (A) represents —O— or —S—.

In one embodiment, in formula (A), both R¹⁶ represent a hydrogen atom and at least one R¹⁷ is other than a hydrogen atom, or both R¹⁷ represent a hydrogen atom and at least one R is other than a hydrogen atom.

In another embodiment, in formula (A), at least one R¹⁶ is other than a hydrogen atom and at least one R¹⁷ is other than a hydrogen atom.

In one embodiment, if present in formula (A), each R¹⁸ represents a hydrogen atom and n is 2.

In one embodiment, if present in formula (A), each R¹⁹ represents a hydrogen atom and q is 2, 3 or 4.

In one embodiment, if present in formula (A), each R²⁰ represents a hydrogen atom and t is 2, 3 or 4.

In a fourth embodiment, R⁷ is represented by a group of formula (A) wherein
X^{A} represents N;
each X^{B} independently represents a single bond or —C(R¹⁴)₂—, provided that at least one X^{B} represents —C(R¹⁴)₂—;
each R¹⁴ independently represents a hydrogen atom or a methyl group;
X^{C} represents —O—;
each R¹⁶ independently represents a hydrogen or a halogen (e.g. fluorine) atom or a C₁-C₄ alkyl, C₁-C₄ haloalkyl (e.g. trifluoromethyl) or phenyl group, or two R¹⁶ may together represent a —(CH₂)_{q}— group, wherein q is 2, 3 or 4; and
each R¹⁷ independently represents a hydrogen or a halogen (e.g. fluorine) atom or a C₁-C₄ alkyl, C₁-C₄ haloalkyl (e.g. trifluoromethyl) or phenyl group, or two R¹⁷ may together represent a —(CH₂)ₜ— group, wherein t is 2, 3 or 4.

In a fifth embodiment, R⁷ is represented by a group of formula (A) wherein
X^{A} represents N;
each X^{B} independently represents a single bond or —C(R¹⁴)₂—, provided that at least one X^{B} represents —C(R¹⁴)₂—;
each R¹⁴ independently represents a hydrogen atom or a methyl group;
X^{C} represents —O—;
each R¹⁶ independently represents a hydrogen or a fluorine atom or a methyl, trifluoromethyl or phenyl group, or two R¹⁶ may together represent a —(CH)₂)_{q}— group, wherein q is 2, 3 or 4; and
each R¹⁷ independently represents a hydrogen or a fluorine atom or a methyl, trifluoromethyl or phenyl group, or two R¹⁷ may together represent a —(CH₂)ₜ— group, wherein t is 2, 3 or 4.

In a sixth embodiment, R⁷ represents a hydrogen or a halogen atom (e.g. fluorine, chlorine or bromine), hydroxyl, cyano, NR⁹R¹⁰ (e.g. methylamino or dimethylamino), or a C₁-C₆, or C₁-C₄, or C₁-C₂ alkoxy or benzyloxy group.

In a particular embodiment of the invention, R⁷ represents any one of the following moieties or is selected from a group containing any two or more of such moieties: hydrogen, bromine and chlorine atoms and (1-methylcyclopropyl)methoxy, (2,2-difluorocyclopropyl)methoxy, (2,6-dimethyloxan-4-yl)oxy, (2-methylcyclopropyl)methoxy, (2R)-2-(methoxymethyl)pyrrolidin-1-yl, (2R)-2-methylmorpholin-4-yl, (2R)-2-phenylmorpholin-4-yl, (2R,5R)-2,5-dimethylmorpholin-4-yl, (2R,6R)-2,6-dimethylmorpholin-4-yl, (2S)-2-methylmorpholin-4-yl, (2S)-2-phenylmorpholin-4-yl, (2S,5S)-2,5-dimethylmorpholin-4-yl, (3,3-difluorocyclobutyl)methoxy, (3R)-oxolan-3-yloxy, (3S)-oxolan-3-yloxy, (4,4-difluorocyclohexyl)oxy, (4-methyl-1,3-thiazol-2-yl)methoxy, (dimethyl-1,3-oxazol-4-yl)methoxy, (E)-2-cyclopropylethenyl, 1-(pyridin-2-yl)ethoxy, 1,4-oxazepan-4-yl, 1-cyclopentylethoxy, 1-cyclopropylethoxy, 1H-pyrazol-1-yl, 1-phenylethoxy, 2-(2-methylpropy)morpholin-4-yl, 2-(methoxymethyl)morpholin-4-yl, 2-(propan-2-yl)morpholin-4-yl, 2-(trifluoromethyl)morpholin-4-yl, 2,2-diethylmorpholin-4-yl, 2,2-dimethylmorpholin-4-yl, 2,2-dimethylpyrrolidin-1-yl, 2,5-dimethylmorpholin-4-yl, 2,6-dimethylthiomorpholin-4-yl, 2-cyano-morpholin-4-yl, 2-cyclopropylethyl, 2-cyclopropylmorpholin-4-yl, 2-ethyl-2-methylmorpholin-4-yl, 2-ethylmorpholin-4-yl, 2-ethylthiomorpholin-4-yl, 2-methoxyethoxy, 2-methylmorpholin-4-yl, 2-methylphenyl, 2-methylpiperidin-1-yl, 2-methylthiomorpholin-4-yl, 2-oxa-6-azaspiro[3.5]nonan-(6-yl, 3-(1H-pyrazol-1-yl)piperidin-1-yl, 3,3-difluoropiperidin-1-yl, 3,3-difluoropyrrolidin-1-yl, 3,3-dimethylpyrrolidin-1-yl, 3,5-dimethyl-1H-pyrazol-1-yl, 3-ethoxypiperidin-1-yl, 3-methoxypiperidin-1-yl, 3-methoxypyrrolidin-1-yl, 3-methylmorpholin-4-yl, 3-methylphenyl, 3-methylpiperidin-1-yl, 4-(cyclopropylmethoxy)piperidin-1-yl, 4-(methoxymethyl)piperidin-1-yl, 4,4-difluorocyclohex-1-en-1-yl, 4,4-difluorocyclohexyl, 4,4-difluoropiperidin-1-yl, 4-fluoropiperidin-1-yl, 4-methoxypiperidin-1-yl, 4-methylphenyl, 4-methylpiperidin-1-yl, 4-oxa-7-azaspiro[2.5]octan-7-yl, 5-oxa-8-azaspiro[3.5]nonan-8-yl, 6-azaspiro[2.5]octan-6-yl, 6-oxa-9-azaspiro[4.5]decan-9-yl, 8-oxa-3-azabicyclo[3.2.1]octan-3-yl, azepan-1-yl, azetidin-1-yl, benzyloxy, cyclobutoxy, cyclohex-1-en-1-yl, cyclohexyl, cyclohexylmethoxy, cyclohexyloxy, cyclopent-1-en-1-yl, cyclopentyl, cyclopentylmethoxy, cyclopentyloxy, cyclopropylmethoxy, ethylamino, morpholin-4-yl, N-(1,3-dioxolan-2-ylmethyl)-N-methyl-amino, N-(2,2-difluoroethyl)-N-methyl-amino, N-(2,2-dimethyloxan-4-yl)-N-methyl-amino, N-(cyclohexylmethyl)-N-ethylamino, N-(cyclopropylmethyl)-4-N-(oxolan-2 ylmethyl)-amino, N-(cyclopropylmethyl)-amino, N,N-diethylamino, N-[(2-methoxyphenyl)methyl]-N-methyl-amino, N-[(3-chlorophenyl)methyl]-N-methyl-amino, N-cyclopropyl-N-methyl-amino, N-ethyl-4-N-(furan-2-ylmethyl)-amino, N-ethyl-4-N-[(1-methyl-1H-pyrazol-4-yl)methyl]-amino, N-ethyl-N-(oxan-4-ylmethyl)-amino, N-ethyl-N-methyl-amino, N-methyl-4-[(5-methyl-1,2-oxazol-3-yl)methyl]-amino, N-methyl-N-(oxan-2-ylmethyl)-amino, N-methyl-N-(oxan-4-yl)-amino, N-methyl-N-(propan-2-yl)-amino, N-methyl-N-(pyridin-2-ylmethyl)-amino, octahydrocyclopenta[b]morpholin-4-yl, oxan-2-ylmethoxy, oxan-3-ylmethoxy, oxan-4-ylmethoxy, oxan-4-yloxy, oxolan-3-ylmethoxy, pentan-3-yloxy, phenyl, piperidin-1-yl, prop-1-en-2-yl, propan-2-yl, pyridin-3-yl, pyridin-4-yl, pyrrolidin-1-yl, hydroxyl, cyano, methoxy, ethoxy, benzyloxy, N-methylamino and N-dimethylamino.

As stated above, either R⁸ represents a saturated 3- to 8-membered ring system which may comprise at least one ring heteroatom (e.g. one, two, three or four ring heteroatoms) independently selected from nitrogen, oxygen and sulphur, wherein the 3- to 8-membered ring system is optionally substituted by at least one substituent (e.g. one, two, three or four substituents) independently selected from halogen (e.g. fluorine, chlorine, bromine or iodine), hydroxyl and C₁-C₆, or C₁-C₄, or C₁-C₂ alkyl, or R⁸ represents a C₁-C₆, or C₁-C₄, or C₁-C₂ alkyl group optionally substituted by at least one substituent (e.g. one, two, three or four substituents) independently selected from phenyl and C₃-C₆ cycloalkyl, the cycloalkyl group itself being optionally substituted by at least one C₁-C₆, or C₁-C₄, or C₁-C₂ alkyl group.

This R⁸ saturated 3- to 8-membered ring system may comprise one or more (e.g. one, two, three or four) ring heteroatoms independently selected from nitrogen, oxygen and sulphur. The ring system may be monocyclic or bicyclic in which the two or more rings are fused, bridged or spiro, and is attached to the nitrogen atom of the central ring system through a ring carbon atom. Examples of such ring systems include cyclopropyl, cyclobutyl, cyclopentyl, cyclohexyl, tetrahydrofuranyl, tetrahydropyranyl, azetidinyl, pyrrolidinyl, piperidinyl, morpholinyl, thiomorpholinyl, azepanyl, oxazepanyl and bicyclo[2.2.1]heptanyl.

In an embodiment of the invention, R⁸ represents a saturated 4- to 7-membered ring system which may comprise at least one ring heteroatom (e.g. one, two, three or four ring heteroatoms) independently selected from nitrogen, oxygen and sulphur, wherein the 4- to 7-membered ring system is optionally substituted by at least one substituent (e.g. one, two, three or four substituents) independently selected from halogen (e.g. fluorine, chlorine, bromine or iodine), hydroxyl and C₁-C₂ alkyl, or R⁸ represents a C₁-C₆, or C₁-C₄, or C₁-C₂ alkyl group optionally substituted by at least one substituent (e.g. one, two, three or four substituents) independently selected from phenyl and C₃-C₆ cycloalkyl, the cycloalkyl group itself being optionally substituted by at least one (e.g. one or two independently selected) C₁-C₂ alkyl groups.

In one aspect, R⁸ represents a C₄-C₆ cycloalkyl group optionally substituted by at least one substituent (e.g. one, two, three or four substituents) independently selected from fluorine, hydroxyl and methyl.

In another aspect, R⁸ represents a C₁-C₂ alkyl group optionally substituted by at least one substituent (e.g. one, two, three or four substituents) independently selected from phenyl and C₃-C₆ cycloalkyl, the cycloalkyl group itself being optionally substituted by one or two independently selected C₁-C₂ alkyl groups.

In a particular embodiment of the invention, R⁸ represents any one of the following moieties or is selected from a group containing any two or more of such moieties:
(i) cyclohexyl,
(ii) cycloheptyl,
(iii)cyclopentyl,
(iv) 4,4-(difluoro)cyclohexyl,
(v) 4-tetrahydropyranyl,
(vi) cyclobutyl,
(vii) (2-methyl)cyclohexyl,
(viii) n-butyl,
(ix) phenethyl,
(x) 2-(hydroxyl)cyclohexyl,
(xi) (cyclopropyl)ethyl,
(xii) (cyclobutyl)ethyl,
(xiii) 3-tetrahydropyranyl,
(xiv) 3,3-(dimethyl)butyl,
(xv) bicyclo[2.2.1]heptanyl,
(xvi) (cyclopentyl)methyl,
(xvii) (ethyl)cyclopropylmethyl, and
(xviii) 2,2-(dimethyl)cyclopropylmethyl.

As stated above, R¹² and R¹³ each independently represent a hydrogen atom or a C₁-C₆, or C₁-C₄, or C₁-C₂ alkyl (e.g. methyl) group.

In an embodiment of the invention, R¹² and R¹³ both represent a methyl group.

As stated above, R¹⁴ represents a hydrogen atom or a C₁-C₆, or C₁-C₄, or C₁-C₂ alkyl (e.g. methyl) group.

In an embodiment of the invention, R¹⁴ represents a methyl group.

In an embodiment of the invention, the compound of formula (I) is one in which;
Q represents —SO₂—, —SO₂NH— or —SO₂N(CH₃)—;
X⁴ represents N;
X⁵ represents CR⁵;
X⁶ represents CR⁶;
X⁷ represents N;
R¹ and R² each independently represent a hydrogen atom;
R⁵ represents a hydrogen or halogen atom, or a C₁-C₆ alkyl group;
R⁶ represents a hydrogen atom or a C₁-C₆ alkyl group;
R⁸ represents a C₄-C₆ cycloalkyl group optionally substituted by at least one substituent independently selected from fluorine, hydroxyl and methyl; and
R³ and R⁹ to R¹³ are as defined above.

Examples of compounds of the invention include:
7-(benzenesulfonyl)-5-cyclohexyl-5H-pyrrolo[2,3-b]pyrazin-6-amine,
5-cycloheptyl-7-[(4-methylbenzene)sulfonyl]-5H-pyrrolo[2,3-b]pyrazin-6-amine,
5-cycloheptyl-7-[(4-methylbenzene)sulfonyl]-5H-pyrrolo[2,3-b]pyrazin-6-amine,
5-cyclopentyl-7-[(4-methylbenzene)sulfonyl]-5H-pyrrolo[2,3-b]pyrazin-6-amine,
7-[(4-chlorobenzene)sulfonyl]-5-cyclohexyl-5H-pyrrolo[2,3-b]pyrazin-6-amine,
5-cyclohexyl-7-[(4-fluorobenzene)sulfonyl]-5H-pyrrolo[2,3-b]pyrazin-6-amine,
5-cyclohexyl-7-{[4-(propan-2-yloxy)benzene]sulfonyl}-5H-pyrrolo[2,3-b]pyrazin-6-amine,
5-cyclohexyl-7-(thiophene-2-sulfonyl)-5H-pyrrolo[2,3-b]pyrazin-6-amine,
3-(benzenesulfonyl)-1-cyclohexyl-1H-pyrrolo[3,2-b]pyridin-2-amine,
1-cyclopentyl-3-[(4-methylbenzene)sulfonyl]-1H-pyrrolo[3,2-b]pyridin-2-amine,
1-cyclohexyl-3-[(4-methylbenzene)sulfonyl]-1H-pyrrolo[2,3-b]pyridin-2-amine,
7-(cyclohexanesulfonyl)-5-cyclohexyl-5H-pyrrolo[2,3-b]pyrazin-6-amine,
5-(4,4-difluorocyclohexyl)-7-[(4-methoxybenzene)sulfonyl]-5H-pyrrolo[2,3-b]pyrazin-6-amine,
1-(4,4-difluorocyclohexyl)-3-[(4-methoxybenzene)sulfonyl]-1H-pyrrolo[2,3-b]pyridin-2-amine,
3-(benzenesulfonyl)-1-cyclohexyl-1H-pyrrolo[2,3-b]pyridin-2-amine,
3-(benzenesulfonyl)-1-cyclohexyl-1H-pyrrolo[2,3-b]pyridin-2-amine,
3-(benzenesulfonyl)-1-(4,4-difluorocyclohexyl)-1H-pyrrolo[2,3-b]pyridin-2-amine,
7-(benzenesulfonyl)-5-cyclohexyl-5H-pyrrolo[3,2-d]pyrimidin-6-amine,
3-(benzenesulfonyl)-1-cyclohexyl-1H-pyrrolo[2,3-c]pyridin-2-amine,
3-(benzenesulfonyl)-1-(4,4-difluorocyclohexyl)-1H-pyrrolo[3,2-b]pyridin-2-amine,
1-(4,4-difluorocyclohexyl)-3-[(4-methoxybenzene)sulfonyl]-1H-pyrrolo[3,2b]pyridin-2-amine,
3-(benzenesulfonyl)-1-cyclohexyl-1H-pyrrolo[3,2-c]pyridin-2-amine,
methyl N-[7-(benzenesulfonyl)-5-cyclohexyl-5H-pyrrolo[2,3-b]pyrazin-6-yl]carbamate,
3-(benzenesulfonyl)-1-(4,4-difluorocyclohexyl)-6-methyl-1H-pyrrolo[2,3-b]pyridin-2-amine,
7-(benzenesulfonyl)-5-cyclohexyl-4-methoxy-5H-pyrrolo[3,2-d]pyrimidin-6-amine,
5-(benzenesulfonyl)-3-cloro-7-cyclohexyl-7H-pyrrolo[2,3-c]pyridazin-6-amine,
5-(benzenesulfonyl)-7-cyclohexyl-7H-pyrrolo[2,3-c]pyridazin-6-amine,
7-(benzenesulfonyl)-5-(4,4-difluorocyclohexyl)-4-ethoxy-5H-pyrrolo[3,2-d]pyrimidin-6-amine,
7-(benzenesulfonyl)-4-(benzyloxy)-5-(4,4-difluorocyclohexyl)-5H-pyrrolo[3,2d]pyrimidin-6-amine,
6-amino-5-(4,4-difluorocyclohexyl)-7-(phenylsulfonyl)-5H-pyrrolo[3,2-d]pyrimidin-4-ol,
7-(benzenesulfonyl)-4-chloro-5-(4,4-difluorocyclohexyl)-5H-pyrrolo[3,2-d]pyrimidin-6-amine,
7-(benzenesulfonyl)-5-(4,4-difluorocyclohexyl)-4-N-methyl-5H-pyrrolo[3,2-d]pyrimidine-4,6-diamine,
7-(benzenesulfonyl)-5-cyclohexyl-4-N,4-N-dimethyl-5H-pyrrolo[3,2-d]pyrimidine-4,6-diamine,
7-(benzenesulfonyl)-5-cyclohexyl-4-methoxy-5H-pyrrolo[3,2-d]pyrizidin-6-amine,
3-(benzenesulfonyl)-1-cyclohexyl-7-methoxy-1H-pyrrolo[2,3-c]pyridin-2-amine,
6-amino-7-(benzenesulfonyl)-5-cyclohexyl-5H-pyrrolo[3,2-d]pyrimidine-4-carbonitrile,
5-cyclohexyl-7-(2-fluorobenzenesulfonyl)-4-methoxy-2-methyl-5H-pyrrolo[3,2-d]pyrimidin-6-amine,
5-cyclohexyl-7-(3-fluorobenzenesulfonyl)-4-methoxy-2-methyl-5H-pyrrolo[3,2-d]pyrimidin-6-amine,
7-(benzenesulfonyl)-4-methoxy-5-(oxan-4-yl)-5H-pyrrolo[3,2-d]pyrimidin-6-amine,
6-amino-5-cyclohexyl-N-phenyl-5H-pyrrolo[2,3-b]pyrazine-7-sulfonamide,
6-amino-5-cyclohexyl-N-(pyridin-3-yl)-5H-pyrrolo[2,3-b]pyrazine-7-sulfonamide,
5-cyclobutyl-7-(phenylsulfonyl)-5H-pyrrolo[2,3-b]pyrazin-6-amine,
5-(2-methylcyclohexyl)-7-(phenylsulfonyl)-5H-pyrrolo[2,3-b]pyrazin-6-amine,
5-butyl-7-(phenylsulfonyl)-5H-pyrrolo[2,3-b]pyrazin-6-amine,
5-phenethyl-7-(phenylsulfonyl)-5H-pyrrolo[2,3-b]pyrazin-6-amine,
2-(6-amino-7-(phenylsulfonyl)-5H-pyrrolo[2,3-b]pyrazin-5-yl)cyclohexanol,
5-(2-cyclopropylethyl)-7-(phenylsulfonyl)-5H-pyrrolo[2,3-b]pyrazin-6-amine,
5-(4,4-difluoro-cyclohexyl)-7-(phenylsulfonyl)-5H-pyrrolo[2,3-b]pyrazin-6-amine,
5-(2-cyclobutylethyl)-7-(phenylsulfonyl)-5H-pyrrolo[2,3-b]pyrazin-6-amine,
7-(phenylsulfonyl)-5-(tetrahydro-2H-pyran-3-yl)-5H-pyrrolo[2,3-b]pyrazin-6-amine,
5-(3,3-dimethylbutyl)-7-(phenylsulfonyl-5H-pyrrolo[2,3-b]pyrazin-6-amine,
5-(1R*,2R*,4S*)bicyclo[2.2.1]heptan-2-yl)-7-(phenylsulfonyl)-5H-pyrrolo[2,3-b]pyrazin-6-amine,
5-(cyclopentylmethyl)-7-(phenylsulfonyl)-5H-pyrrolo[2,3-b]pyrazin-6-amine,
5-((1-ethylcyclopropyl)-methyl)-7-(phenylsulfonyl)-5H-pyrrolo[2,3-b]pyrazin-6-amine,
5-((2,2-dimethylcyclopropyl)methyl)-7-(phenylsulfonyl)-5H-pyrrolo[2,3-b]pyrazin-6-amine,
5-cyclohexyl-7-(piperidin-1-ylsulfonyl)-5H-pyrrolo[2,3-b]pyrazin-6-amine,
5-cyclohexyl-7-(pyrrolidin-1-ylsulfonyl)-5H-pyrrolo[2,3-b]pyrazin-6-amine,
6-amino-5-cyclohexyl-N-propyl-5H-pyrrolo[2,3-b]pyrazine-7-sulfonamide,
6-amino-5-cyclohexyl-N-methyl-N-propyl-5H-pyrrolo[2,3-b]pyrazine-7-sulfonamide,
5-cyclohexyl-7-(morpholinosulfonyl)-5H-pyrrolo[2,3-b]pyrazin-6-amine,
5-cyclohexyl-7-((4-methylpiperidin-yl)sulfonyl)-5H-pyrrolo[2,3-b]pyrazin-6-amine,
5-cyclohexyl-7-((4-methylpiperazin-1-yl)sulfonyl)-5H-pyrrolo[2,3-b]pyrazin-6-amine,
5-cyclohexyl-7-((3-methoxyazetidin-1-yl)sulfonyl)-5H-pyrrolo[2,3-b]pyrazin-6-amine,
5-cyclohexyl-7-((4-ethoxypiperidin-1-yl)sulfonyl)-5H-pyrrolo[2,3b]pyrazin-6-amine,
5-cyclohexyl-7-((4,4-methylpiperidin-1-yl)sulfonyl)-5H-pyrrolo[2,3-b]pyrazin-6-amine,
5-cyclohexyl-7-((3-methylpyrrolidin-1-yl)sulfonyl)-5H-pyrrolo[2,3-b]pyrazin-6-amine,
5-cyclohexyl-7-((2-methylpyrrolidin-1-yl)sulfonyl)-5H-pyrrolo[2,3-b]pyrazin-6-amine,
5-cyclohexyl-7-((4,4-difluoropiperidin-1-yl)sulfonyl)-5H-pyrrolo[2,3b]pyrazin-6-amine,
6-amino-N-benzyl-5-cyclohexyl-5H-pyrrolo[2,3-b]pyrazine-7-sulfonamide,
6-amino-N,5-dicyclohexyl-N-methyl-5H-pyrrolo[2,3-b]pyrazine-7-sulfonamide,
5-cyclohexyl-7-(1,4-oxazepane-4-sulfonyl)-5H pyrrolo[2,3-b]pyrazin-6-amine,
5-cyclohexyl-7-(4-methoxypiperidine-1-sulfonyl)-5H pyrrolo[2,3-b]pyrazin-6-amine,
6-amino-N-(cyclobutylmethyl)-5-cyclohexyl-5H-pyrrolo[2,3-b]pyrazine-7-sulfonamide,
5-cyclohexyl-7-(3,3-dimethylpyrrolidine-1-sulfonyl)-5H-pyrrolo[2,3-b]pyrazin-6-amine,
5-cyclohexyl-7-(2,6-dimethylmorpholine-4-sulfonyl)-5H-pyrrolo[2,3-b]pyrazin-6-amine,
7-(azepane-1-sulfonyl)-5-cyclohexyl-5H-pyrrolo[2,3-b]pyrazin-6-amine,
5-cyclohexyl-7-(thiomorpholine-4-sulfonyl)-5H-pyrrolo[2,3-b]pyrazin-6-amine,
N-(1-{6-amino-5-cyclohexyl-5H-pyrrolo[2,3-b]pyrazine-7-sulfonyl}piperidin-4-yl)-N-methylacetamide,
6-amino-5-cyclohexyl-N-(oxetan-3-ylmethyl)-5H-pyrrolo[2,3-b]pyrazine-7-sulfonamide,
7-(4-benzylpiperidine-1-sulfonyl)-5-cyclohexyl-5H-pyrrolo[2,3-b]pyrazin-6-amine,
6-amino-5-cyclohexyl-N-(3,3,3-trifluoropropyl)-5H-pyrrolo[2,3-b]pyrazine-7-sulfonamide,
5-cyclohexyl-7-(4-phenylpiperidine-1-sulfonyl)-5H-pyrrolo[2,3-b]pyrazin-6-amine,
6-amino-5-cyclohexyl-N-(2-phenylethyl)-5H-pyrrolo[2,3-b]pyrazine-7-sulfonamide,
5-cyclohexyl-7-(4-phenoxypiperidine-1-sulfonyl)-5H-pyrrolo[2,3-b]pyrazin-6-amine,
5-cyclohexyl-7-(3-phenylpyrrolidine-1-sulfonyl)-5H-pyrrolo[2,3-b]pyrazin-6-amine,
5-cyclohexyl-7-[4-(trifluoromethyl)piperidine-1-sulfonyl]-5H-pyrrolo[2,3-b]pyrazin-6-amine,
5-cyclohexyl-7-[3-(methoxymethyl)pyrrolidine-1-sulfonyl]-5H-pyrrolo[2,3-b]pyrazin-6-amine,
6-amino-5-cyclohexyl N-(cyclopropylmethyl)-5H-pyrrolo[2,3-b]pyrazine-7-sulfonamide,
6-amino-5-cyclohexyl-N-(2-methoxyethyl)-5H-pyrrolo[2,3-b]pyrazine-7-sulfonamide,
5-cyclohexyl-7-(3-methoxypyrrolidine-1-sulfonyl)-5H-pyrrolo[2,3-b]pyrazin-6-amine,
5-cyclohexyl-7-(3,3-dimethylpiperidine-1-sulfonyl)-5H-pyrrolo[2,3-b]pyrazin-6-amine,
1-{6-amino-5-cyclohexyl-5H-pyrrolo[2,3-b]pyrazine-7-sulfonyl}piperidin-4-ol,
5-cyclohexyl-7-(1,2,3,4-tetrahydroisoquinoline-2-sulfonyl)-5H-pyrrolo[2,3-b]pyrazin-6-amine,
6-amino-N-(butan-2-yl)-5-cyclohexyl-5H-pyrrolo[2,3-b]pyrazine-7-sulfonamide,
6-amino-5-cyclohexyl-N-(oxolan-2-ylmethyl)-5H-pyrrolo[2,3-b]pyrazine-7-sulfonamide,
5-cyclohexoyl-7-(2,3-dihydro-1H-isoindole-2-sulfonyl)-5H-pyrrolo[2,3-b]pyrazin-6-amine,
5-cyclohexyl-7-{4-[(4-fluorophenyl)carbonyl]piperazine-1-sulfonyl}-5H-pyrrolo[2,3-b]pyrazin-6-amine,
5-cyclohexyl-7-(3-phenoxyazetidine-1-sulfonyl)-5H-pyrrolo[2,3-b]pyrazin-6-amine,
5-cyclohexyl-7-[3-(piperidin-1-yl)azetidine-1-sulfonyl]-5H-pyrrolo[2,3-b]pyrazin-6-amine,
5-cyclohexyl-7-[3-(1H-pyrazol-1-yl)azetidine-1-sulfonyl]-5H-pyrrolo[2,3-b]pyrazin-6-amine,
5-cyclohexyl-7-(3-methylpiperidine-1-sulfonyl)-5H-pyrrolo[2,3-b]pyrazin-6-amine,
6-amino-5-cyclohexyl-N-[2-(1,3-thiazol-2-yl)ethyl]-5H-pyrrolo[2,3-b]pyrazine-7-sulfonamide,
8-{6-amino-5-cyclohexyl-5H-pyrrolo[2,3-b]pyrazine-7-sulfonyl}-8-azabicyclo[3.2.1]octan-3-ol,
5-cyclohexyl-7-[4-(2,2,2-trifluoroethyl)-piperazine-1-sulfonyl]-5H-pyrrolo[2,3-b]pyrazin-6-amine,
(1-{6-amino-5-cyclohexyl-5H-pyrrolo[2,3-b]pyrazine-7-sulfonyl}piperidin-4-yl)methanol,
5-cyclohexyl-7-[4-(cyclopropylmethoxy)piperidine-1-sulfonyl]-5H pyrrolo[2,3-b]pyrazin-6-amine,
5-cyclohexyl-7-[(4-methoxybenzene)-sulfonyl]-5H pyrrolo[2,3-b]pyrazin-6-amine,
5-cyclohexyl-7-(cyclopropanesufonyl)-5H-pyrrolo[2,3-b]pyrazin-6-amine,
5-cyclohexyl-7-[(3-fluorobenzene)sulfonyl]-5H-pyrrolo[2,3-b]pyrazin-6-amine,
5-cyclohexyl-7-[(2-fluorobenzene)sulfonyl]-5H-pyrrolo[2,3-b]pyrazin-6-amine,
5-cyclohexyl-7-[3-methoxybenzene)-sulfonyl]-5H-pyrrolo[2,3-b]pyrazin-6-amine,
4-{6-amino-5-cyclohexyl-5H-pyrrolo[2,3-b]pyrazine-7-sulfonyl}benzonitrile,
7-[(3-chloro-4-methoxybenzene)-sulfonyl]-5-cyclohexyl-5H-pyrrolo[2,3-b]pyrazin-6-amine,
5-cyclohexyl-7-(6-methoxypyridine-3-sulfonyl)-5H-pyrrolo[2,3-b]pyrazin-6-amine,
5-cyclohexyl-7-{[4-(trifluoromethoxy)-benzene]sulfonyl}-5H pyrrolo[2,3-b]pyrazin-6-amine,
5-cyclohexyl-7-(2,3-dihydro-1,4-benzodioxine-6-sulfonyl)-5H-pyrrolo[2,3-b]pyrazin-6-amine,
5-cyclohexyl-7-{[4-(difluoromethoxy)-benzene]sulfonyl}-5H-pyrrolo[2,3-b]pyrazin-6-amine,
and pharmaceutically acceptable salts of any one thereof.

It should be noted that each of the chemical compounds listed above represents a particular and independent aspect of the invention.

The present invention further provides a process for the preparation of a compound of formula (I) or a pharmaceutically acceptable salt thereof as defined above which comprises,

(a) when NR¹R² represents NH₂, reacting a compound of formula

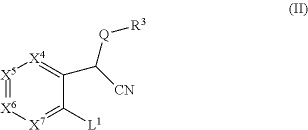

wherein L¹ represents a leaving group (e.g. a halogen atom or trifluoromethanesulphonate group) and X⁴, X⁵, X⁶, X⁷, Q and R³ are as defined in formula (I), with a compound of formula (III), H₂NR⁸, or a salt thereof (e.g. a hydrochloride salt) wherein R⁸ is as defined in formula (I); or
(b) when NR¹R² represents NH₂, reacting a compound of formula

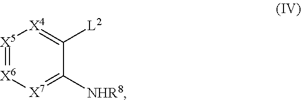

wherein L² represents a leaving group (e.g. a halogen atom or trifluoromethanesulphonate group) and X⁴, X⁵, X⁶, X⁷ and R⁸ are as defined in formula (I), with a compound of formula

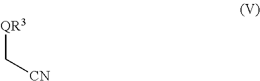

wherein Q and R³ are as defined in formula (I);
wherein any of compounds (II), (III), (IV) or (V) may optionally be protected;
and optionally thereafter carrying out one or more of the following procedures:
removing any protecting groups
converting a compound of formula (I) into another compound of formula (I)
forming a pharmaceutically acceptable salt.

In process (a) the compound of formula (II) may conveniently be combined with an amine of formula (III) or a salt thereof in the presence of a base such as triethylamine or ethylbis(propan-2-yl)amine, in a solvent such as anhydrous N-methylpyrrolidone, to arrive at a compound of formula (I). Typically the reaction mixture is heated, e.g. to around 170° C. under microwave irradiation.

Process (b) may conveniently be carried out by combining the compound of formula (IV) with the substituted acetonitrile of formula (V) in the presence of a base such as sodium hydride or sodiobis(trimethylsilyl)amine, and a metal catalyst such as Pd(o), typically where the metal catalyst is in the form of a transition metal complex such as tetrakis(triphenylphosphine) palladium and/or di-tert-butyl[dichloro({di-tert-butyl[4-(dimethylamino)phenyl]-phosphaniumyl})palladio][4-(dimethylamino)phenyl] phosphanium, in a solvent such as 1,2-dimethoxyethane, dioxane or 2-methyloxalane, typically where the solvent is anhydrous, to arrive at a compound of formula (I). Typically the reaction mixture is heated, e.g. to around 70-150° C. under conventional heating or microwave irradiation. Optionally the Pd(o) catalyst may be formed in situ, e.g. from Pd(I) acetate and 2,8,9-tris(2-methylpropyl)-2,5,8,9-tetraaza-1-phosphabicyclo[3.3.3]undecane.

Compounds of formula (II) may be prepared by reacting a compound of formula

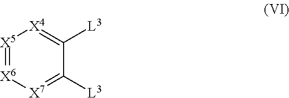

wherein each L³ independently represents a leaving group (e.g. a halogen atom or trifluoromethanesulphonate group) and X⁴, X⁵, X⁶ and X⁷ are as defined above, with a compound of formula (V) as defined above. The reaction is conveniently carried out in the presence of a base such as sodium hydride, and a metal catalyst such as Pd(o), typically where the metal catalyst is in the form of a transition metal complex such as tetrakis(triphenylphosphine) palladium, in a solvent such as anhydrous 1,2-dimethoxyethane, to arrive at a compound of formula (II) which may or may not be isolated. Typically the reaction mixture is heated, e.g. to around 70-140° C. under conventional heating or microwave irradiation.

In one embodiment, a compound of formula (I) or a salt or a protected form thereof, may be converted into another compound of formula (I) or a salt or a protected form thereof.

For example, a compound of formula (I) or a salt or a protected form thereof, where R¹ and R² are both hydrogen, may be converted into another compound of formula (I) or a salt or a protected form thereof where one or both of R¹ and R² are not hydrogen, typically by treatment with a compound of formula R¹-L and/or R²-L, where R¹ and R² are as previously defined but not hydrogen and L is as previously defined for L¹.

In one convenient procedure, a compound of formula (I) or a salt thereof, where R¹ and R² are both hydrogen, may be combined with a compound of formula (C₁-C₆ alkyl)-L′, where L′ is a leaving group such as a chlorine, bromine or iodine atom, in the presence of a base such as butyllithium, and a solvent such as anhydrous THF. Typically the reaction mixture is cooled, e.g. to about 0° C.

In another convenient procedure, a compound of formula (I) or a salt thereof, where R¹ and R are both hydrogen, may be combined with a compound of formula L″-COO—(C₁-C₆ alkyl), where L″ is a leaving group such as a chlorine, bromine or iodine atom, in the presence of a base such as ethylbis(propan-2-yl)amine, and a solvent such as anhydrous dichloromethane. Typically the reaction mixture is heated, e.g. to about 30-50° C.

Substituents R⁴, R⁵, R⁶ and R⁷ may also be modified and/or replaced after the formation of a compound of formula (I).

For example, where R⁴, R⁵, R⁶ or R⁷ represents a halogen atom selected from chlorine, bromine or iodine, the halogen atom may be substituted to arrive at an alternate compound of formula (I).

Where the new substituent requires carbon-carbon bond formation, in a convenient procedure a compound of formula (I) where, for example, R⁷ represents a chlorine, bromine or iodine atom, may be combined with a boric acid derivative such as R^{7a}—B(OH)₂, R^{7a}—B(pinacole ester) or R^{7a}—BF₃—K⁺ where R^{7a} represents the replacement R⁷ bonded to the boron atom via a carbon-boron bond, in the presence of a base such as potassium carbonate, caesium carbonate or potassium phosphate, and a metal catalyst such as Pd(o), typically where the metal catalyst is in the form of a transition metal complex such as tetrakis(triphenylphosphine) palladium or di-tert-butyl[dichloro({di-tert-butyl[4-(dimethylamino)phenyl]-phosphaniumyl})palladio][4-(dimethylamino)phenyl] phosphanium. A solvent such as a dioxane/water mixture may be used and the reaction mixture is typically heated, e.g. to around 100-160° C. under conventional heating or microwave irradiation.

Where the new substituent requires carbon-nitrogen bond formation, in a convenient procedure a compound of formula (I) where, for example, R⁷ represents a chlorine, bromine or iodine atom, may be combined with a primary or secondary amine of formula R^{7a}H, where R^{7a} represents the replacement R⁷ and includes a nitrogen atom through which the R^{7a} group is to be bonded to the remainder of the compound of formula (I). Examples of R^{7a}H include morpholine, piperidine, pyrrolidine and substituted derivatives thereof. Optionally, the reaction is performed in the presence of an additional base such as triethylamine or ethylbis(propan-2-yl)amine. A solvent such as ethanol, anhydrous tetrahydrofuran, anhydrous N-methylpyrrolidone or anhydrous N,N-dimethylformamide may be used and the reaction mixture is typically heated, e.g. to around 60-200° C. under conventional heating or microwave irradiation.

In a similar procedure, where it is desired to form a carbon-nitrogen bond to a suitable ring nitrogen of a heterocyclic amine, a compound of formula (I) where, for example, R⁷ represents a chlorine, bromine or iodine atom, may be combined with the heterocyclic amine in the presence of a base such as sodium hydride and a solvent such as anhydrous N,N-dimethylformamide. The reaction mixture is typically heated, e.g. to around 200° C. under conventional heating or microwave irradiation.

Where the new substituent requires carbon-oxygen bond formation, in a convenient procedure a compound of formula (I) where, for example, R⁷ represents a chlorine, bromine or iodine atom, may be combined with the desired alcohol in the presence of a base such as sodium hydride and a solvent such as anhydrous tetrahydrofuran. The reaction mixture is typically heated, e.g. to around 60-120° C. under conventional heating or microwave irradiation.

The above procedures to substitute R⁴, R⁵, R⁶ or R⁷, where R⁴, R⁵, R⁶ or R⁷ initially represents a leaving group such as a chlorine, bromine or iodine atom, may also be applied to synthesise suitably substituted compounds of formula (IV) or (VI) prior to their reaction with compounds of formula (V). Likewise, they may be applied to the intermediates of formula (II) to replace substituents prior to reaction with an amine of formula (III) or a salt thereof.

The compounds of formula (V) where Q is —SO₂— may conveniently be synthesised by reacting a compound of formula R³SO₂Cl with a compound of formula ClCH₂CN, in the presence of a reducing agent such as disodium sulfite, and a base such as sodium hydrogen carbonate, in a solvent such as a water/propan-2-ol or water/tetrahydrofuran mixture. The reaction mixture is typically heated, e.g. to around 100-120° C. under conventional heating or microwave irradiation.

In an alternate procedure, the compounds of formula (V) where Q is —SO₂— and R³ is an amino group attached to the remainder of the compound via the nitrogen atom of the amino group, may be synthesised by reacting the corresponding amine R³H with cyanomethanesulfonyl chloride in the presence of a base such as triethylamine and a solvent such as anhydrous dichloromethane. Typically, the reaction is performed at a temperature of from 20-30° C.

Compounds of formulae (II), (IV), (V) and (VI) are either commercially available, are known in the literature or may be prepared using known techniques.

As already indicated, in the above processes certain functional groups such as phenol, hydroxyl or amino groups in the reagents may need to be protected by protecting groups. Thus, the preparation of the compounds of formula (I) may involve, at an appropriate stage, the introduction and f/or removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3rd edition, T.W. Greene and P.G.M. Wuts, Wiley-Interscience (1999).

The compounds of formula (I) above may be converted to a pharmaceutically acceptable salt thereof, preferably an acid addition salt such as a hydrochloride, hydrobromide, benzenesulphonate (besylate), saccharin (e.g. monosaccharin), trifluoroacetate, sulphate, nitrate, phosphate, acetate, fumarate, maleate, tartrate, lactate, citrate, pyruvate, succinate, valerate, propanoate, butanoate, malonate, oxalate, 1-hydroxy-2-napthoate (xinafoate), methanesulphonate or p-toluenesulphonate salt.

In one aspect of the invention, compounds of formula (I) may bear one or more radiolabels. Such radiolabels may be introduced by using radiolabel-containing reagents in the synthesis of the compounds, or may be introduced by coupling the compounds to chelating moieties capable of binding to a radioactive metal atom. Such radiolabeled versions of the compounds may be used, for example, in diagnostic imaging studies.

Unless stated otherwise, any atom specified herein may also be an isotope of said atom. For example, the term “hydrogen” encompasses ¹H, ²H and ³H. Similarly carbon atoms are to be understood to include ¹²C, ¹³C and ¹⁴C, nitrogen atoms are to be understood to include ¹⁴N and ¹⁵N, and oxygen atoms are to be understood to include ¹⁶O, ¹⁷O and ¹⁸O.

In a further aspect of the invention, compounds of formula (I) may be isotopically labelled. As used herein, an “isotopically labelled” compound is one in which the abundance of a particular nuclide at a particular atomic position within the molecule is increased above the level at which it occurs in nature.

In a still further aspect, the invention provides prodrugs of the compounds of formula (I). The term “prodrug” as used herein refers to a derivative of an active form of a compound which derivative, when administered to a subject, is gradually converted to the active form to produce a better therapeutic response and/or a reduced toxicity level. In general, prodrugs will be functional derivatives of the compounds disclosed herein which are readily convertible in vivo into the compound from which it is notionally derived. Prodrugs include, without limitation, acyl esters, carbonates, phosphates, and urethanes. These groups are exemplary and not exhaustive, and one skilled in the art could prepare other known varieties of prodrugs. Prodrugs may be, for example, formed with available hydroxy, thiol, amino or carboxyl groups. For example, available NH₂ groups in the compounds of the invention may be acylated using an activated acid in the presence of a base, and optionally, in inert solvent (e.g. an acid chloride in pyridine). Conventional procedures for the selection and preparation of suitable prodrugs are described, for example, in “Design of Prodrugs” ed. H. Bundgaard, Elsevier, 1985.

Compounds of formula (I) and their salts may be in the form of hydrates or solvates which form an aspect of the present invention. Such solvates may be formed with common organic solvents, including but not limited to, alcoholic solvents e.g. methanol, ethanol or isopropanol.

Where compounds of formula (I) are capable of existing in stereoisomeric forms, it will be understood that the invention encompasses the use of all geometric and optical isomers (including atropisomers) of the compounds and mixtures thereof including racemates. The use of tautomers and mixtures thereof also forms an aspect of the present invention. Enantiomerically pure forms are particularly desired.

Compounds of formula (I) and their salts may be amorphous or in a polymorphic form or a mixture of any of these, each of which forms an aspect of the present invention.

The compounds of formula (I) and their pharmaceutically acceptable salts have activity as pharmaceuticals, in particular as GPR43 receptor agonists and/or as positive allosteric modulators of the GPR43 receptor. Accordingly, they may be used in the treatment of obesity; diabetes, in particular diabetes mellitus such as diabetes mellitus type 1, diabetes mellitus type 2 and gestational diabetes; metabolic syndrome; atherosclerosis; irritable bowel syndrome; and autoimmune diseases including inflammatory bowel disease (e.g. Crohn's disease and ulcerative colitis), rheumatoid arthritis and systemic lupus. The compounds may also be used in the treatment of asthma, liver fibrosis, non-alcoholic steatohepatitis (NASH), neuroinflammation, multiple sclerosis and colorectal cancer.

As used herein, the term “obesity” refers to a person who has a body mass index (BMI) of greater than or equal to 30 kg/m². The BMI may be calculated by dividing a patient's weight in kilograms by the square of their height in metres (kg/m²).

Thus, the present invention provides a compound of formula (I) or a pharmaceutically acceptable salt thereof, as hereinbefore defined, for use in therapy, in particular for the treatment of conditions whose development or symptoms are linked to GPR43 receptor activity.

The present invention also provides the use of a compound of formula (I) or a pharmaceutically acceptable salt thereof, as hereinbefore defined, for the preparation of a medicament for the treatment of conditions whose development or symptoms are linked to GPR43 receptor activity.

In the context of the present specification, the terms “therapy” and “treatment” also include “prophylaxis” unless there are specific indications to the contrary. The terms “therapeutic”, “therapeutically” and “treating” should be construed accordingly.

Prophylaxis is expected to be particularly relevant to the treatment of persons who have suffered a previous episode of, or are otherwise considered to be at increased risk of, the disorder or condition in question. Persons at risk of developing a particular disorder or condition generally include those having a family history of the disorder or condition, or those who have been identified by genetic testing or screening to be particularly susceptible to developing the disorder or condition or those in the prodromal phase of a disorder.

In particular, the compounds of the invention (including pharmaceutically acceptable salts) may be used in the treatment of obesity and/or diabetes (including diabetes mellitus such as diabetes mellitus type 1, diabetes mellitus type 2 and gestational diabetes).

In one embodiment, the compounds of the invention (including pharmaceutically acceptable salts) may be used in the treatment of obese diabetics, including those suffering from diabetes mellitus type 1, diabetes mellitus type 2 or gestational diabetes.

In another embodiment, the compounds of the invention (including pharmaceutically acceptable salts) may be used in the treatment of inflammatory bowel disease.

The present invention also provides a method of treating obesity, diabetes (including diabetes mellitus such as diabetes mellitus type 1, diabetes mellitus type 2 and gestational diabetes) or inflammatory bowel disease, which comprises administering to a patient in need thereof a therapeutically effective amount of a compound of formula (I) or a pharmaceutically acceptable salt thereof as hereinbefore defined.

For the above-mentioned therapeutic uses the dosage administered will, of course, vary with the compound employed, the mode of administration, the treatment desired and the disorder indicated. For example, the daily dosage of the compound of the invention, if inhaled, may be in the range from 0.05 micrograms per kilogram body weight (μg/kg) to 100 micrograms per kilogram body weight (μg/kg). Alternatively, if the compound is administered orally, then the daily dosage of the compound of the invention may be in the range from 0.01 micrograms per kilogram body weight (μg/kg) to 100 milligrams per kilogram body weight (mg/kg), preferably from 0.01 to 1 mg/kg body weight

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier.

Therefore the present invention further provides a pharmaceutical composition comprising a compound of formula (I) or a pharmaceutically acceptable salt thereof, as hereinbefore defined, in association with a pharmaceutically acceptable adjuvant, diluent or carrier.

The invention still further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

Conventional procedures for the selection and preparation of suitable pharmaceutical formulations are described in, for example, “Pharmaceutics—The Science of Dosage Form Design”, M. E. Aulton, Churchill Livingstone, 1988.

Pharmaceutically acceptable adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the present invention may be administered orally, parenterally, by inhalation spray, rectally, nasally, buccally, vaginally or via an implanted reservoir. Oral administration is preferred. The pharmaceutical compositions of the invention may contain any conventional non-toxic pharmaceutically acceptable adjuvants, diluents or carriers. The term parenteral as used herein includes subcutaneous, intracutaneous, intravenous, intramuscular, intra-articular, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. The suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable diluents and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant.

The pharmaceutical compositions of this invention may be orally administered in any orally acceptable dosage form including, but not limited to, capsules, tablets, powders, granules, and aqueous suspensions and solutions. These dosage forms are prepared according to techniques well-known in the art of pharmaceutical formulation. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavouring and/or colouring agents may be added.

The pharmaceutical compositions of the invention may also be administered in the form of suppositories for rectal administration. These compositions can be prepared by mixing the active ingredient with a suitable non-irritating excipient which is solid at room temperature but liquid at the rectal temperature and therefore will melt in the rectum to release the active ingredient. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilising or dispersing agents known in the art.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The compounds of the invention (that is, compounds of formula (I) and pharmaceutically acceptable salts thereof) may also be administered in conjunction with other compounds used for the treatment of the above conditions, for example, biguanide drugs (for example Metformin), insulin (synthetic insulin analogues), oral antihyperglycemics (these are divided into prandial glucose regulators and alpha-glucosidase inhibitors) and sulfonylureas (for example, glimepiride, glibenclamide (glyburide), gliclazide, glipizide, gliquidone, chloropropamide, tolbutamide, acetohexamide, glycopyramide, carbutamide, glibonuride, glisoxepid, glybuthiazole, glibuzole, glyhexamide, glymidine, glypinamide, phenbutamide, tolcylamide and tolazamide). Preferably the sulfonylurea is glimepiride or glibenclamide (glyburide).

Alternatively, the compounds of the invention may be administered in combination with a dipeptidyl peptidase-4 (DPP IV) inhibitor (for example, alogliptin); or a phosphodiesterase-4 (PDE4) inhibitor (for example, rolipram, roflumilast or apremilast); or bupropion/naltrexone (“Contrave”); or lorcaserin hydrochloride (“Lorqess”); or phentermine/topiramate (“Qsymia”).

The present invention will now be further explained by reference to the following illustrative examples. In the illustrative examples, the compounds synthesised are both named and illustrated structurally. Whilst every effort has been made to ensure that the chemical names and the chemical structures are consistent, if any inconsistencies occur the illustrated chemical structure should be taken to be correct, unless the illustrated chemical structure is chemically impossible.

The methods used for the synthesis of the compounds of the invention are illustrated by the general schemes below and the preparative examples that follow. The starting materials and reagents used in preparing these compounds are available from commercial suppliers. These general schemes are merely illustrative of methods by which the compounds of this invention can be synthesised, and various modifications to these schemes can be made and will be suggested to one skilled in the art having referred to this disclosure.

### EXPERIMENTAL

Nuclear magnetic resonance (NMR) spectra were recorded at 400 MHz or 300 MHz as stated and at 300.3K unless otherwise stated; the chemical shifts (δ) are reported in parts per million. Spectra were recorded using a Bruker 400 AVANCE instrument fitted with a 5 mm BBFO probe with instrument controlled by Bruker TopSpin 2.1 software, or by a Bruker 400 AVANCE-III instrument fitted with a 5 mm BBFO probe with instrument controlled by Bruker TopSpin 3.0 software, or by a Bruker 300 MHz AVANCE II instrument fitted with a 5 mm DUL probe with instrument controlled by Bruker TopSpin 1.3 software.

Purity was assessed using one or more of the following:
UPLC with UV (photodiode array) detection over a wide range of wavelengths, normally 220-450 nm, using a Waters Acquity UPLC system equipped with Acquity UPLC BEH or HSS C18 columns (2.1 mm id×50 mm long) operated at 50 or 60° C. Mobile phases typically consisted of acetonitrile or methanol mixed with water containing either 0.1% formic acid or 0.025% ammonia. Mass spectra were recorded with a Waters SQD single quadrupole mass spectrometer using atmospheric pressure ionisation.
UPLC with UV (photodiode array) detection over a wide range of wavelengths, normally 200-500 nm, using a Waters Acquity H-Class UPLC system controlled by Empower-2 software. Mass spectra were recorded with a Waters SQD single quadrupole mass spectrometer using electro spray ionization. Mobile phase consisted of 5 mm Ammonium Acetate containing 0.1% formic acid in Water and Acetonitrile using Acquity UPLC BEH or HSS C18 columns (2.1 mm id×50 mm long).
LCMS with UV (photodiode array) detection over a wide range of wavelengths, normally 200-500 nm and the detection was also proceed at wavelength 260 nm and 80 bandwidth, using Shimandzu Nexera LCMS-2020 system controlled by Lab Solution software. Mass spectra were recorded with a single quadrupole mass spectrometer using electro spray ionization. Mobile phase consisted of 20 mm Ammonium Acetate mixed with water and Methanol using Waters X-bridge column (C18, 5 μm, 4.6 mm id×150 mm).
LCMS with UV (photodiode array) detection over a wide range of wavelengths, normally 200-500 nm, using Waters ZQ-2000 system controlled by Empower-1 software. Mass spectra were recorded with a Waters ZQ single quadrupole mass spectrometer using electro spray ionization. Mobile phases consisted of 0.1% Ammonia mixed with water and Acetonitrile using Waters X-bridge column (C18, 5 μm, 4.6 mm id×150 mm).

Compounds were purified using normal phase chromatography on silica, using Biotage or Isolute KP-Sil cartridges or Kinesis Telos Silica cartridges, or on basic silica, using Biotage or Isolute KP-NH cartridges, or by reverse phase chromatographic methods, using Biotage or Isolute KP-C18-HS cartridges or by SCX-2 catch-release cartridges, or by Preparative HPLC.

Preparative HPLC was performed using one or more of the following:
Agilent Technologies 1100 Series system or a Waters autopurification LC/MS system typically using Waters 19 mm id×250 mm long C18 columns such as XBridge or SunFire 5 μm materials at room temperature.
Shimadzu Preparative HPLC system typically using 19 mm id×150 mm long C18 columns 5 μm or 20 mm id×250 mm long C8 columns 5 μm materials at room temperature. Shimadzu Preparative HPLC system controlled by LC-Solution software.

Mobile phases typically consisted of acetonitrile or methanol mixed with water containing either 0.1% formic acid or 0.1% ammonia, unless stated otherwise.

Room temperature in the following examples means the temperature ranging from 20° C. to 25° C.

The abbreviations used in the specific examples have the following meanings:
Ac acetyl
aq aqueous
Bn, Bzl benzyl
BOC, Boc tert-butoxycarbonyl
bp boiling point,
br broad (spectral)
Bu, n-Bu normal (primary) butyl
t-Bu tert-butyl
Bz benzoyl
CBZ, Cbz benzyloxycarbonyl
CD₂Cl₂ deuterated dichloromethane
CDCl₃ deuterated chloroform
CHCl₃ chloroform
m-CPBA meta-chloroperoxybenzoic acid
Cy cyclohexyl
δ chemical shift in ppm downfield from tetramethylsilane
d day(s); doublet (spectral);
DCE 1,2-dichloroethane
DCM dichloromethane
DMAP 4-(N,N-dimethylamino)pyridine
DME 1,2-dimethoxyethane
DMF dimethylformamide
DMSO dimethyl sulfoxide
DMSO-d₆ perdeuterated dimethyl sulfoxide
DPPF 1,1′-bis(diphenylphosphanyl) ferrocene
ES electrospray
Et ethyl
H-frit Biotage Phase Separator (Part #120-1908-F)
h hour(s)
HPLC high-performance liquid chromatography
Hz hertz
L litre(s)
LDA lithium diisopropylamide
μ micro
m multiplet (spectral); metre(s); milli
M molar (moles per litre); mega
Me methyl
mg milligram
MgSO₄ magnesium sulfate
min minute(s); minimum
mL millilitre
mmol millimoles
mmolar millimolar (millimoles per liter)
mol mole(s); molecular (e.g. in mol. wt.)
mp melting point
Ms, mesyl methylsulfonyl
MS mass spectrometry
MTBE methyl tert-butyl ether
MW molecular weight
m/z mass-to-charge ratio
NaHCO₃ sodium bicarbonate; sodium hydrogen carbonate
NaHMDS sodium hexamethyldisilazane
nm nanometer(s)
NMP N-methylpyrrolidone
NMR nuclear magnetic resonance
Pd(amphos)₂Cl₂ Bis(di-tert-butyl(4-dimethylaminophenyl)phosphine) dichloropalladium(II)
Ph phenyl
PMB p-methoxybenzyl
ppm part(s) per million
Pr, n-Pr propan-1-yl
iPr isopropyl
q quartet (spectral)
rt room temperature
s singlet (spectral); second(s)
Sat. saturated
t triplet (spectral)
t time; temperature in units of degrees Celsius (° C.)
TEA triethylamine
Tf, trifyl trifluoromethanesulfonyl
TFA trifluoroacetic acid
TFAA trifluoroacetic anhydride
THF tetrahydrofuran
THP tetrahydropyran-2-yl
TMEDA N,N,N′,N′-tetramethyl-1,2-ethylenediamine
Ts, tosyl para-toluenesulfonyl
UV ultraviolet

### 1. Intermediates

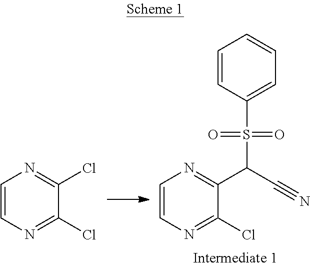 [SMILES: Clc1nccnc1Cl ; N#CC(c1nccnc1Cl)S(=O)(=O)c1ccccc1]

### Intermediate 1 2-(benzenesulfonyl)-2-(3-chloropyrzin-2-yl)acetonitrile

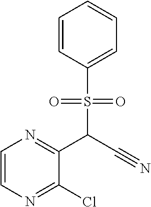 [SMILES: N#CC(c1nccnc1Cl)S(=O)(=O)c1ccccc1]

To a stirred solution of 2,3-dichloropyrazine (CAS 4858-85-9; 1.4 mL, 13 mmol) and 2-(benzenesulfonyl)acetonitrile (CAS 7605-28-9; 24 g, 13 mmol) in DMSO (8 mL) was added DBU (4.1 mL, 27 mmol). The reaction was subjected to microwave irradiation at 100° C. for 45 mins. The reaction mixture was diluted with water and brine and then extracted with ethyl acetate. The aqueous phase was extracted further with DCM. The combined organics were dried over MgSO₄ and concentrated in vacuo. The crude product was loaded onto a plug of silica (10 g) and eluted using 0-100% EtOAc/petroleum ether. The product fractions were concentrated in vacuo to afford the title compound.

¹H NMR (400 MHz, DCM-d₂) δ ppm 6.01 (s, 1H) 7.62-7.72 (m, 2H) 7.83-7.91 (m, 3H) 8.51-8.59 (m, 2H)

MS ES⁺: 294

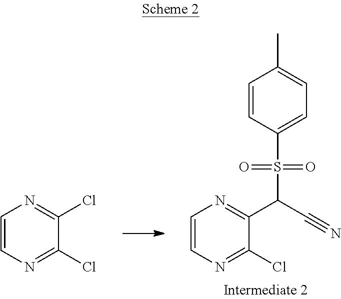 [SMILES: Clc1nccnc1Cl ; Cc1ccc(S(=O)(=O)C(C#N)c2nccnc2Cl)cc1]

### Intermediate a 2-(3-chloropyrazin-2-yl)-2-(4-methylbenzenesulfonyl)acetonitrile

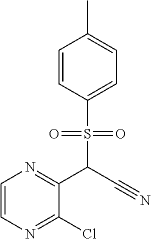 [SMILES: Cc1ccc(S(=O)(=O)C(C#N)c2nccnc2Cl)cc1]

To a stirred solution of 2,3-dichloropyrazine (CAS 4858-85-9; 360 μL, 3-4 mmol) and 2-(4-methylbenzenesulfonyl)acetonitrile (CAS 5697-44-9; 736 mg, 3.8 mmol) in acetonitrile (7 mL) was added DBU (620 μL, 4.1 mmol). The reaction was heated in a microwave at 80° C. for 30 min. The reaction mixture was evaporated to dryness and purified by column chromatography (C18-silica 0-30% Acetonitrile+0.05% NH₃/Water+0.1% NH₃) to afford the title compound.

¹H NMR (400 MHz, DMSO-d₆) δ ppm 2.46 (s, 3H) 7.00 (s, 1H) 7.50 (d, J=1 Hz, 2H) 7.62 (d, J=1 Hz, 2H) 8.64-8.75 (m, 2H)

MS ES⁺: 308

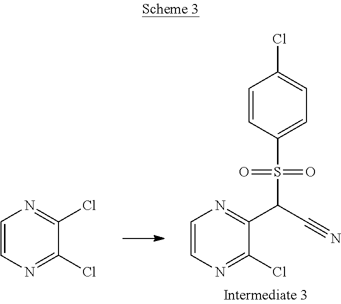 [SMILES: Clc1nccnc1Cl ; N#CC(c1nccnc1Cl)S(=O)(=O)c1ccc(Cl)cc1]

### Intermediate 3 2-(4-chlorobenzenesulfonyl)-2-(3-chloropyrazin-2-yl)acetonitrile

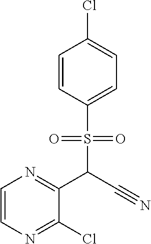 [SMILES: N#CC(c1nccnc1Cl)S(=O)(=O)c1ccc(Cl)cc1]

To a stirred solution of 2,3-dichloropyrazine (CAS 4858-85-9; 156 μL, 1.50 mmol) and 2-(4-chlorophenylsulfonyl)acetonitrile (CAS 1851-09-8; 323 mg, 1.50 mmol) in DMF (1 mL) was added DBU (452 μL, 3.00 mmol). The reaction was heated in a microwave at 100° C. for 30 min. The reaction mixture was diluted with ammonium chloride solution, extracted with EtOAc/tetrahydrofuran (2:1), the combined organics dried (H frit) and evaporated to dryness. The crude product was purified by column chromatography silica (silica, 0-100% EtOAc/petroleum ether) to afford the title compound.

MS ES⁺: 328

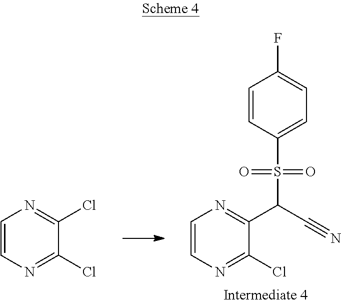 [SMILES: Clc1nccnc1Cl ; N#CC(c1nccnc1Cl)S(=O)(=O)c1ccc(F)cc1]

### Intermediate 4 2-(3-chloropyrazin-1-yl)-2-(4-fluorobenzenesulfonyl)acetonitrile

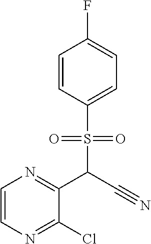 [SMILES: N#CC(c1nccnc1Cl)S(=O)(=O)c1ccc(F)cc1]

Prepared as described for 2-(4-chlorobenzenesulfonyl)-2-(3-chloropyrazin-2-yl)acetonitrile (Intermediate 3), to a stirred solution of 2,3-dichloropyrazine (CAS 4858-85-9; 156 μL, 1.50 mmol) and 2-(4-fluorophenylsulfonyl)acetonitrile (CAS 32083-66-2; 299 mg, 1.50 mmol) in DMF (1 mL) was added DBU (452 μL, 3.00 mmol).

The reaction was heated in a microwave at 100° C. for 30 min. The crude product was purified by column chromatography (silica, 0-50% EtOAc/petroleum ether) to afford the title compound.

MS ES⁺: 312

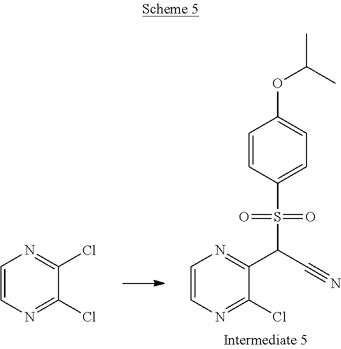 [SMILES: Clc1nccnc1Cl ; CC(C)Oc1ccc(S(=O)(=O)C(C#N)c2nccnc2Cl)cc1]

### Intermediate 5 2-(3-chloropyrazin-2-yl)-2-[4-(propan-2-yloxy)benzenesulfonyl]acetonitrile

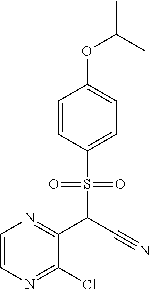 [SMILES: CC(C)Oc1ccc(S(=O)(=O)C(C#N)c2nccnc2Cl)cc1]

Prepared as described for 2-(4-Chlorobenzenesulfonyl)-2-(3-chloropyrazin-2-yl)acetonitrile (Intermediate 3), to a stirred solution of 2,3-dichloropyrazine (CAS 4858-85-9; 156 μL, 1.50 mmol) and 2-(4-isopropoxyphenylsulfonyl)acetonitrile (CAS 886499-39-4; 359 mg, 1.50 mmol) in DMF (1 mL) was added DBU (452 μL, 3.00 mmol). The reaction was then heated in a microwave at 100° C. for 30 min. The crude product was purified by column chromatography (silica, 0-40% EtOAc/petroleum ether) to afford the title compound.

MS ES⁺: 352

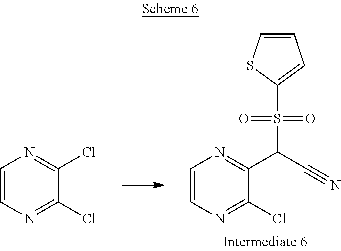 [SMILES: Clc1nccnc1Cl ; N#CC(c1nccnc1Cl)S(=O)(=O)c1cccs1]

### Intermediate 6 2-(3-chloropyrazin-2-yl)-2-(thiophene-2-sulfonyl)acetonitrile

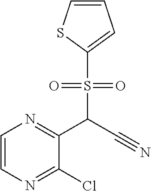 [SMILES: N#CC(c1nccnc1Cl)S(=O)(=O)c1cccs1]

Prepared as described for 2-(4-chlorobenzenesulfonyl)-2-(3-chloropyrazin-2-yl)acetonitrile (Intermediate 3), to a stirred solution of 2,3-dichloropyrazine (CAS 4858-85-9; 156 μL 1.50 mmol) and 2-(thiophen-2-ylsulfonyl)acetonitrle (CAS 175137-62-9; 281 mg, 1.50 mmol) in DMF (1 mL) was added DBU (452 μL, 3.00 mmol). The reaction was heated in a microwave at 100° C. for 30 min then 125° C. for 30 min. The crude product was purified by column chromatography (C18-silica, 0-30% Acetonitrile+0.05% NH₃/Water+0.1% NH₃) to afford the title compound.

MS ES⁺: 300.

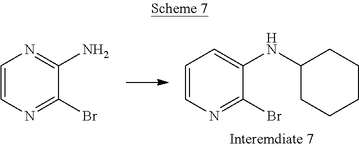 [SMILES: Nc1nccnc1Br ; Brc1ncccc1NC1CCCCC1]

### Intermediate 7 2-bromo-N-cyclohexylpyridin-3-amine

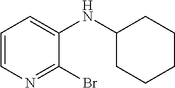 [SMILES: Brc1ncccc1NC1CCCCC1]

To a stirred solution of cyclohexanone (CAS 108-94-1; 851 mg, 8.67 mmol) and 2-bromopyridin-3-amine (CAS 39856-58-1; 500 mg, 2.89 mmol) in DCM (8 mL) at 0° C. under N₂ was added TiCl₄ solution (1M in DCM, 3.18 mL, 3.18 mmol) dropwise. The reaction mixture was allowed to stir for 2 hours at rt and then cooled to 0° C. Sodium triacetoxyborohydride (1.8 g, 8.67 mmol) was added portionwise and then the reaction allowed to warm to rt and stirred over a weekend. The reaction mixture was quenched slowly into water and then extracted with DCM. The organic phase was separated and concentrated in vacuo. The crude product was purified by column chromatography (silica, 0-40% EtOAc/petroleum ether) to afford the title compound.

MS ES⁺: 255

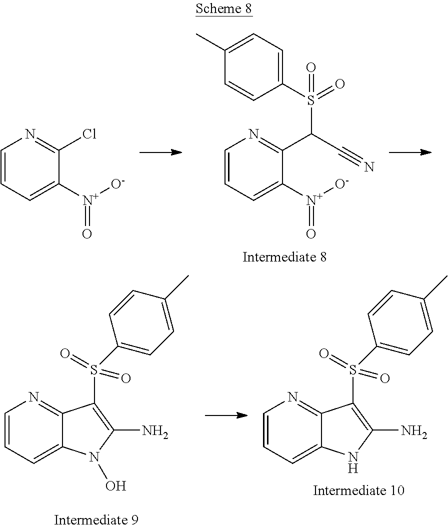 [SMILES: O=[N+]([O-])c1cccnc1Cl ; Cc1ccc(S(=O)(=O)C(C#N)c2ncccc2[N+](=O)[O-])cc1 ; Cc1ccc(S(=O)(=O)c2c(N)n(O)c3cccnc23)cc1 ; Cc1ccc(S(=O)(=O)c2c(N)[nH]c3cccnc23)cc1]

### Intermediate 8 2-(4-methylbenzenesulfonyl)-2-(3-nitropyridin-2-yl)acetonitrile

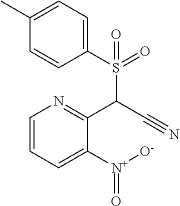 [SMILES: Cc1ccc(S(=O)(=O)C(C#N)c2ncccc2[N+](=O)[O-])cc1]

To a stirred solution of potassium tert-butoxide (3.5 g, 32 mmol) in propan-2-ol (25 mL) at 0° C. was added 2-(4-methylbenzenesulfonyl)acetonitrile (CAS 5697-44-9; 3.69 g, 18 mmol) and the resulting mixture stirred for 30 min. 2-chloro-3-nitropyridine (CAS 5470-18-8; 2.5 g 15.8 mmol) was added and the reaction mixture stirred at 65° C. for 6 h. The reaction mixture was allowed to cool and concentrated in vacuo. The resulting residue was taken up in water and extracted with ethyl acetate. The organic phase was dried (Na₂SO₄) and concentrated in vacuo. The crude product was purified by column chromatography (silica, 25-30% EtOAc/hexane) to afford the title compound.

¹H NMR (4 MHz, DMSO-d₆) δ ppm 2.45 (s, 3H), 6.93 (s, 1H), 7.45-7.55 (m, 2H), 7.55-7.65 (m, 3H), 8.05-8.15 (m, 1H), 8.50-8.60 (m, 1H)

MS ES⁺: 318

### Intermediate 9 2-amino-3-(4-methylbenzenesulfonyl)-1H-pyrrolo[3,2-b]pyridin-1-ol

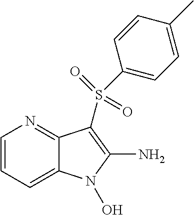 [SMILES: Cc1ccc(S(=O)(=O)c2c(N)n(O)c3cccnc23)cc1]

A stirred suspension of 2-(4-methylbenzenesulfonyl)-2-(3-nitropyridin-2-yl)acetonitrile (Intermediate 8; 1.2 g, 11 mmol) and palladium on carbon (10% w/w) (60 mg, 0.55 mmol) in acetic acid (0.5 mL) and ethyl acetate (50 mL) was placed under an atmosphere of hydrogen. The reaction was stirred at rt for to h. The reaction was filtered and the filtrate concentrated in vacuo. The residue was taken up in water and neutralised with sat. aq. NaHCO₃ solution and then extracted with ethyl acetate. The organics were dried (NaSO₄) and concentrated in vacuo. The crude product was purified by column chromatography (silica, 2-5% MeOH/DCM) to afford the title compound.

¹H NMR (400 MHz, DMSO-d₆) δ ppm 2.30 (s, 3H), 6.90-7.00 (m, 1H), 7.10 (s, 2H), 7.25-7.35 (m, 2H), 7.35-7.45 (m, 1H), 7.85-7.95 (m, 2H), 8.05-8.15 (m, 1H), 11.50 (s, 1H)

MS ES⁺: 304

### Intermediate 10 3-(4-methylbenzenesulfonyl)-1H-pyrrolo[3,2-b]pyridin-2-amine

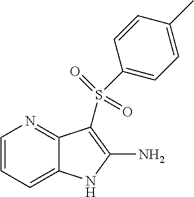 [SMILES: Cc1ccc(S(=O)(=O)c2c(N)[nH]c3cccnc23)cc1]

A stirred suspension of 2-amino-3-(4-methylbenzenesulfonyl)-1H-pyrrolo[3,2-b]pyridin-1-ol (Intermediate 9; 400 mg) and palladium on carbon (10% w/w) (50 mg) in acetic acid (2 mL) and ethyl acetate (10 mL) was placed under an atmosphere of hydrogen at too psi. After 8 h the reaction was diluted with ethyl acetate and filtered. The filtrate was washed with sat. aq. NaHCO₃ solution and the organic phase separated and dried (Na₂SO₄). The organic phase was concentrated in vacuo. The crude product was triturated with hexane and filtered to afford the title compound.

¹H NMR (400 MHz, DMSO-d₆) δ ppm 2.32 (s, 3H) 6.82-6.97 (m, 3H) 7.27-7.40 (m, 3H) 7.86-7.96 (m, 2H) 8.02-8.09 (m, 1H) MS ES⁺: 288

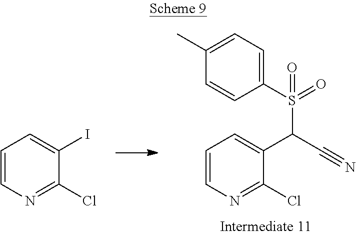 [SMILES: Clc1ncccc1I ; Cc1ccc(S(=O)(=O)C(C#N)c2cccnc2Cl)cc1]

### Intermediate 11 2-(2-chloropyridin-3-yl)-2-(4-methylbenzenesulfonyl)acetonitrile

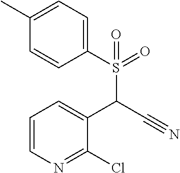 [SMILES: Cc1ccc(S(=O)(=O)C(C#N)c2cccnc2Cl)cc1]

To a stirred solution of 2-chloro-3-iodopyridine (CAS 78607-36-0; 4.9 g, 20.5 mmol) in toluene(15 mL) was added potassium tert-butoxide (2.81 g, 25.0 mmol), Pd₂dba₃ (1.53 g, 1.70 mmol) and 2-(4-methylbenzenesulfonyl)acetonitrile (CAS 5697-44-9; 2.64 g, 14.6 mmol). The reaction was heated at 125° C. for 4 h. The reaction was poured onto ice and extracted with ethyl acetate. The organic phase was separated, dried and concentrated in vacuo. The crude product was purified by column chromatography (silica, 20-22% EtOAc/petroleum ether) to afford the title compound.

¹H NMR (400 MHz, DMSO-d₆) δ ppm 2.45 (s, 3H), 6.76 (s, 1H), 7.50-7.58 (m, 2H), 7.60-7.65 (m, 1H), 7.65-7.75 (m, 2H), 7.90-8.00 (m, 1H), 7.55-7.65 (m, 1H)

MS ES⁺: 307

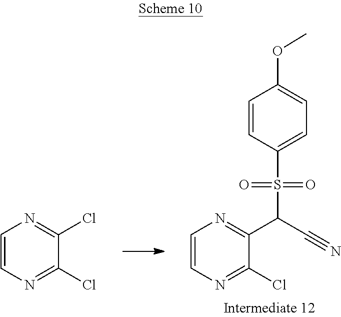 [SMILES: Clc1nccnc1Cl ; COc1ccc(S(=O)(=O)C(C#N)c2nccnc2Cl)cc1]

### Intermediate 12 2-(3-chloropyrazin-2-yl)-2-(4-methoxybenzenesulfonyl)acetonitrle

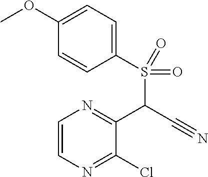 [SMILES: COc1ccc(S(=O)(=O)C(C#N)c2nccnc2Cl)cc1]

A neat mixture of 2,3-dichloropyrazine (CAS 4858-85-9; 0.100 mL, 0.958 mmol), 2-((4-methoxyphenyl)sulfonyl)acetonitrile (CAS 132276-87-0; 220 mg, 0.958 mmol) and DBU (0.289 mL, 1.916 mmol) was heated to 85° C. for 1.5 h. The reaction mixture was treated with dilute citric acid and EtOAc. The phases were separated and the aqueous extracted with EtOAc. The combined organic extracts were then washed with dilute citric acid, water, sat. NaHCO₃, sat. brine, dried (H-frit) and evaporated. The crude material was absorbed onto MgSO₄ from DCM/MeOH and purified by column chromatography (silica, 0-40% EtOAc/petroleum ether) to afford the title compound. ¹H NMR (400 MHz, DCM-d₂) δ ppm 3.96 (s, 3H) 5.99 (s, 1H) 7.10 (d, J=9 Hz, 2H) 7.76 (d, J=9 Hz, 2H) 8.49-8.60 (m, 2H)

MS ES⁺: 324

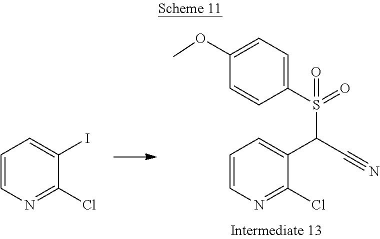 [SMILES: Clc1ncccc1I ; COc1ccc(S(=O)(=O)C(C#N)c2cccnc2Cl)cc1]

### Intermediate 13 2-(2-chloropyridin-3-yl)-2-(4-methoxybenzenesulfonyl)acetontrile

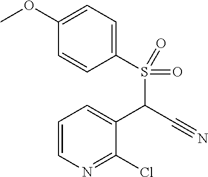 [SMILES: COc1ccc(S(=O)(=O)C(C#N)c2cccnc2Cl)cc1]

To a stirred degassed solution of Pd(Ph₃P)₄ (0.058 g, 0.050 mmol) in anhydrous DME (1.5 mL) under an atmosphere of nitrogen was added a solution of 2-((4-methoxyphenyl)sulfonyl)acetonitrile (CAS 132276-87-0; 0.232 g, 1.10 mmol) and NaH (0.084 g, 2.10 mmol) in anhydrous DME (4 mL). The resulting mixture was stirred at room temperature for to min followed by the addition of 2-chloro-3-iodopyridine (CAS 78607-36-0; 00.239 g, 1 mmol). The reaction was heated in a microwave at 90° C. to 110° C. for 2.5 h. More Pd(Ph₃P)₄ (0.029 g, 0.025 mmol) was added and the reaction heated in a microwave at 115° C. to 120° C. for 1.5 h. The solvent was removed under reduced pressure and the residue was diluted with water, neutralised with 2 M aq. HCl solution and extracted with DCM. The combined organic phases were washed with brine, dried over MgSO₄, filtered and concentrated under reduced pressure. The residue was purified by column chromatography (silica, 10-40% EtOAc/petroleum ether) to afford the title compound.

¹H NMR (400 MHz, CHLOROFORM-d) δ ppm 3.90 (s, 3H) 5.72 (s, 1H) 7.03 (d, J=9 Hz, 2H) 7.33-7.45 (m, 1H) 7.74 (d, J=9 Hz, 2H) 7.89-7.99 (m, 1H) 8.41-8.55 (m, 1H) MS ES⁺: 323

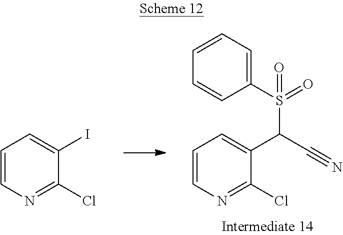 [SMILES: Clc1ncccc1I ; N#CC(c1cccnc1Cl)S(=O)(=O)c1ccccc1]

### Intermediate 2-(benzenesulfonyl)-2-(2-chloropyridin-3-yl)acetonitrile

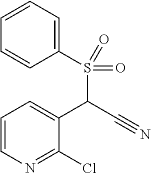 [SMILES: N#CC(c1cccnc1Cl)S(=O)(=O)c1ccccc1]

To a stirred degassed solution of Pd(Ph₃P)₄ (0.116 g, 0.100 mmol) in anhydrous DME (1.5 mL) under an atmosphere of nitrogen was added a solution of 2-(phenylsulfonyl)acetonitrile (0.399 g, 2.20 mmol) and NaH (0.168 g, 4.20 mmol) in anhydrous DME (4 mL). The resulting mixture was stirred at room temperature for to min followed by the addition of 2-chloro-3-iodopyridine (0.479 g, 2.00 mmol). The reaction mixture was heated at 120° C. for 1.5 h. The solvent was removed under reduced pressure and the residue was diluted with water, neutralised with 2 M aq. HCl solution and extracted with DCM. The combined organic phases were washed with brine, dried over MgSO₄, filtered and concentrated under reduced pressure. The residue was purified by column chromatography (silica, 10-40% EtOAc/petroleum ether) to afford the title compound.

¹H NMR (4 MHz, CHLOROFORM-d) δ ppm 5.73 (s, 1H) 7.36-7.45 (m, 1H) 7.56-7.71 (m, 2H) 7.76-7.86 (m, 1H) 7.87-7.94 (m, 2H) 7.95-8.03 (m, 1H) 8.45-8.60 (m, 1H)

MS ES⁺: 293

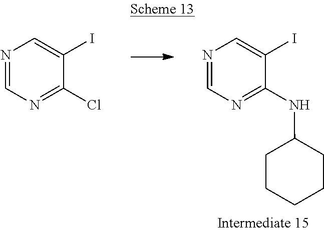 [SMILES: Clc1ncncc1I ; Ic1cncnc1NC1CCCCC1]

### Intermediate 15 N-cyclohexyl-5-iodopyrimidin-4-amine

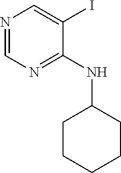 [SMILES: Ic1cncnc1NC1CCCCC1]

A stirred suspension of cyclohexanamine (CAS 108-91-8; 0.114 mL, 0.998 mmol), 4-chloro-5-iodopyrimidine (CAS 63558-65-6; 200 mg, 0.832 mmol) and Cs₂CO₃ (407 mg, 1.248 mmol) in N-methyl-2-pyrrolidinone (2 mL) was heated in a microwave at 100° C. for 1 h. The reaction mixture was poured into water and extracted with EtOAc (×2). The combined extracts were washed with water, dilute citric acid, water, sat. NaHCO₃, sat. brine, dried (H-frit) and evaporated. The crude product was then purified by column chromatography (silica, 0-20% EtOAc/petroleum ether) to afford the title compound. ¹H NMR (400 MHz, CHLOROFORM-d) δ ppm 1.21-1.37 (m, 3H) 1.39-1.54 (m, 2H) 1.63-1.73 (m, 1H) 1.73-1.85 (m, 2H) 1.99-2.12 (m, 2H) 3.96-4.10 (m, 1H) 5.19 (br. s., 1H) 8.44 (s, 1H) 8.46 (s, 1H)

MS ES⁺: 304

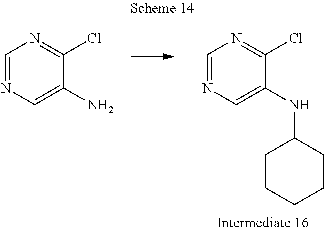 [SMILES: Nc1cncnc1Cl ; Clc1ncncc1NC1CCCCC1]

### Intermediate 16 4-chloro-N-cyclohexylpyrimidin-5-amine

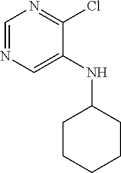 [SMILES: Clc1ncncc1NC1CCCCC1]

To a stirred solution of 4-chloropyrimidin-5-amine (CAS 54660-78-5; 150 mg, 1.16 mmol) and cyclohexanone (CAS 108-94-1; 360 μL, 3.47 mmol) in DCM (5 mL) at 0° C. was added TiCl₄ solution (1.0M in DCM, 1.27 mL, 1.27 mmol). The reaction was stirred at room temperature for 2 h. Sodium triacetoxyborohydride (736 mg, 3.47 mmol) was then added portionwise. Stirring at rt was maintained for 2 h. The reaction mixture was poured into water and extracted with EtOAc (×2). The combined organic extracts were washed with water, sat. NaHCO₃, sat.brine, dried (H-frit) and evaporated. The crude product was purified by column chromatography (silica, 0-15% EtOAc/petroleum ether) to afford the title compound.

¹H NMR (400 MHz, CHLOROFORM-d) δ ppm 1.21-152 (m, 5H) 1.62-1.96 (m, 3H) 1.99-2.17 (m, 2H) 3.29-3.47 (m, 1H) 4.11-4.27 (m, 1H) 8.06 (s, 1H) 8.33 (s, 1H)

MS ES⁺: 212

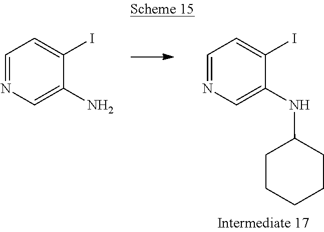 [SMILES: Nc1cnccc1I ; Ic1ccncc1NC1CCCCC1]

### Intermediate 17 N-cyclohexyl-4-iodopyridin-3-amine

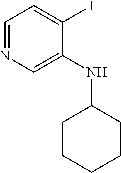 [SMILES: Ic1ccncc1NC1CCCCC1]

To a stirred solution of cyclohexanone (CAS 108-94-1; 1.34 g, 13.6 mmol) and 4-iodopyridin-3-amine (CAS 105752-11-2; 1 g, 4.55 mmol) in DCM (15 mL) at 0° C. under N₂ was added TiCl₄ solution (1.0M in DCM, 5.00 mL, 5.00 mmol) dropwise. The reaction mixture was allowed to stir at rt for 2 hours and then sodium triacetoxyborohydride (2.89 g, 13.6 mmol) was added portionwise. The reaction mixture was allowed to stir at room temperature overnight. The reaction mixture was quenched slowly into water and then extracted with DCM. The organics were separated and concentrated. The crude product was purified by column chromatography (silica, 0-50% EtOAc/petroleum ether) to afford the title compound.

¹H NMR (400 MHz, DMSO-d₆) δ ppm 1.07-1.49 (m, 4H) 1.56-1.76 (m, 4H) 1.89-1.97 (m, 2H) 3.42-3.53 (m, 1H) 4.28 (d, J=8 Hz, 1H) 7.48 (d, J=5 Hz, 1H) 7.65 (d, J=5 Hz, 1H) 7.90 (s, 1H)

MS ES⁺: 303

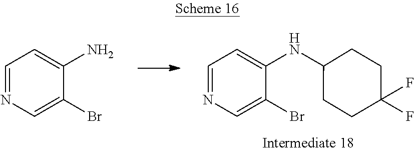 [SMILES: Nc1ccncc1Br ; FC1(F)CCC(Nc2ccncc2Br)CC1]

### Intermediate 18 2-bromo-N-(4,4-difluorocyclohexyl)pyridin-3-amine

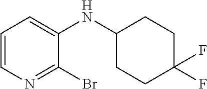 [SMILES: FC1(F)CCC(Nc2cccnc2Br)CC1]

Prepared as described for N-cyclohexyl-4-iodopyridin-3-amine (Intermediate 17), to a stirred solution of 4,4-difluorocyclohexanone (CAS 22515-18-0; 2.33 g, 17.3 mmol) and 2-bromopyridin-3-amine (CAS 39856-58-1; 1 g, 5.78 mmol) in DCM (15 mL) at 0° C. under N₂ was added TiCl₄ solution (1M in DCM, 6.36 mL, 6.36 mmol) dropwise. The reaction was allowed to stir at room temperature for 2 h and then cooled to 0° C. Sodium triacetoxyborohydride (3.68 g, 17.3 mmol) was added portionwise and then the reaction stirred at room temperature for 72 h. The crude product was purified by column chromatography (silica, 0-100% EtOAc/petroleum ether) to afford the title compound.

MS ES⁺: 291

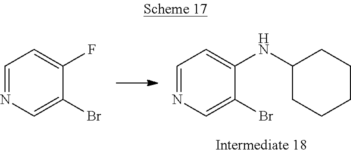 [SMILES: Fc1ccncc1Br ; Brc1cnccc1NC1CCCCC1]

### Intermediate 19 3-bromo-N-cyclohexylpyrldin-4-amine

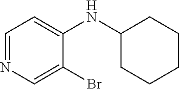 [SMILES: Brc1cnccc1NC1CCCCC1]

A neat mixture of 3-bromo-4-fluoropyridine (200 mg 1.14 mmol) and cyclohexanamine (CAS 108-91-8; 650 μL, 5.68 mmol) was heated in a microwave at 120° C. for 45 min. The reaction mixture was dissolved in EtOAc and washed with water, brine, dried over MgSO₄, filtered and concentrated under reduced pressure. The crude product was purified by column chromatography (silica, 0-50% EtOAc/petroleum ether) to afford the title compound.

¹H NMR (400 MHz, CHLOROFORM-d) δ ppm 1.24-1.49 (m, 4H) 1.60-1.73 (m, 2H) 1.74-1.88 (m, 2H) 1.95-2.16 (m, 2H) 3.18-3.46 (m, 1H) 4.71 (br. s, 1H) 6.48 (d, J=6 Hz, 1H) 8.12 (d, J=6 Hz, 1H) 8.34 (s, 1H)

MS ES⁺: 255

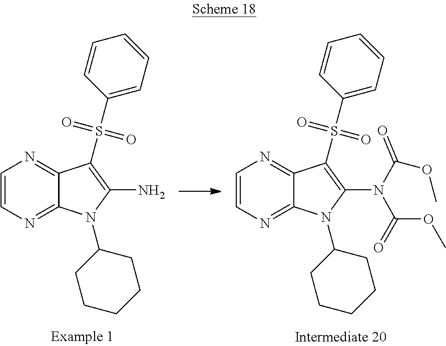 [SMILES: Nc1c(S(=O)(=O)c2ccccc2)c2nccnc2n1C1CCCCC1 ; COC(=O)N(C(=O)OC)c1c(S(=O)(=O)c2ccccc2)c2nccnc2n1C1CCCCC1]

### Intermediate 20 methyl N-[7-(benzenesulfonyl)-5-cyclohexyl-5H-pyrrolo[2,3-b]pyrazin-6-yl]-N-(methoxycarbonyl)carbamate

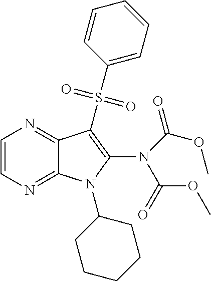 [SMILES: COC(=O)N(C(=O)OC)c1c(S(=O)(=O)c2ccccc2)c2nccnc2n1C1CCCCC1]

To a stirred solution of 7-(benzenesulfonyl)-5-cyclohexyl-5H-pyrrolo[2,3-b]pyrazin-6-amine (Example 1; 0.135 g, 0.38 mmol) in anhydrous THF (5 mL) at −78° C. and under an atmosphere of nitrogen was added dropwise a solution of butyllithium (0.152 mL, 0.380 mmol) in hexanes (2.5 M). The resulting mixture was stirred at −78° C. for 10 min and then quenched at −78° C. by the addition of methyl carbonochloridate (0.294 ml, 3.80 mmol) and allowed to warm to room temperature. The reaction was partitioned between diethyl ether and water. The phases were separated and the aqueous extracted with diethyl ether. The combined organics were dried over MgSO₄, filtered and concentrated in vacuo. Purification was performed by chromatography (preparative HPLC, 40-80% acetonitrile/water (with 0.1% formic acid)) to afford the title compound.

¹H NMR (3 MHz, CHLOROFORM-d) δ ppm 1.18-1.48 (m, 3H) 1.65-1.99 (m, 5H) 2.40-2.67 (m, 2H) 3.76 (s, 6H) 4.04-4.29 (m, 1H) 7.39-7.65 (m, 3H) 8.07-8.25 (m, 2H) 8.38 (d, J=2 Hz, 1H) 8.66 (d, J=2 Hz, 1H)

MS ES⁺: 473

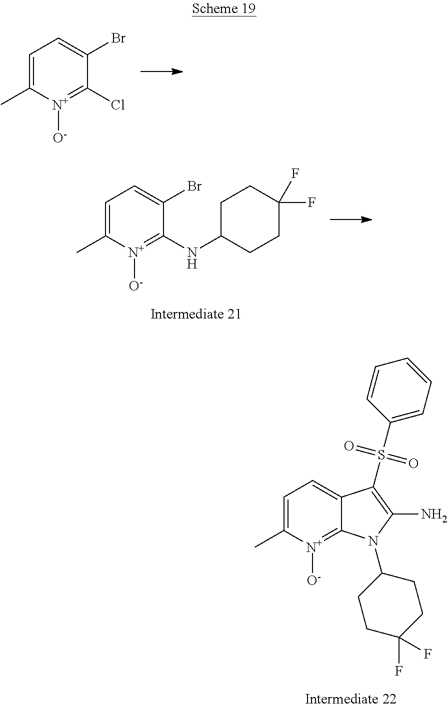

### Intermediate 21 3-bromo-((4,4-difluorocyclohexyl)amino)-6-methylpyridine 1-oxide

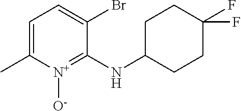 [SMILES: Cc1ccc(Br)c(NC2CCC(F)(F)CC2)[n+]1[O-]]

To a stirred solution of 3-bromo-2-chloro-6-methylpyridine 1-oxide (CAS 185017-76-9; 0.309 & 1.39 mmol) and difluorocyclohexanamine hydrochloride (CAS 675112-70-6; 0.309 g, 1.80 mmol) in NMP (3 mL) was added Cs₂CO₃ (1.22 g, 3.74 mmol) and the resulting mixture was heated at 110° C. to 140° C. for 6 h using a microwave reactor. The mixture was partitioned between ethyl acetate and water. The phases were separated and the aqueous extracted with ethyl acetate (×2) The combined organics were washed with water, brine, dried over MgSO₄, filtered and concentrated under reduced pressure.

The crude product was purified by column chromatography (silica, 20-100% EtOAc/petroleum ether) to afford the title compound.

MS ES⁺: 321

### Intermediate 22 2-amino-1-(4,4-difluorocyclohexyl)-6-methyl-3-(phenylsulfonyl)-1H-pyrrolo[2,3-b]pyridine 7-oxide

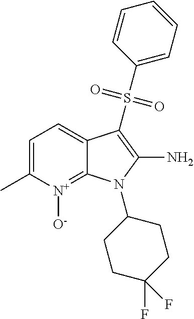 [SMILES: Cc1ccc2c(S(=O)(=O)c3ccccc3)c(N)n(C3CCC(F)(F)CC3)c2[n+]1[O-]]

To a stirred degassed solution of Pd(Ph₃P)₄ (18 mg, 0.016 mmol) in anhydrous DME (1 mL) under an atmosphere of nitrogen was added a solution of 2-(benzenesulfonyl)acetonitrile (CAS 7605-28-9; 62 mg, 0.343 mmol) and NaH, 60% dispersion in oil (26 mg, 0.654 mmol) in anhydrous DME (1 mL). The resulting mixture was stirred at room temperature for 10 min followed by addition of a solution of 3-bromo-2-((4,4-difluorocyclohexyl)amino)-6-methylpyridine 1-oxide (Intermediate 21; 1 mg, 0.311 mmol) in anhydrous DME (1 mL). The reaction mixture was heated at 120° C. for 1.5 h. The solvent was removed under reduced pressure and the residue was diluted with water, neutralised with 2 M aq. HCl solution and extracted with DCM. The combined organic phases were washed with brine, dried over MgSO₄, filtered and concentrated under reduced pressure. The crude product was purified by column chromatography (silica, 0-10% MeOH/DCM) to afford the title compound.

MS ES⁺: 422

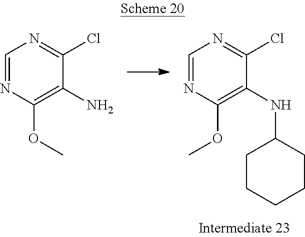

### Intermediate 23 4-chloro-N-cyclohexyl-6-methoxypyrimidin-5-amine

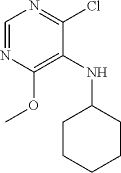 [SMILES: COc1ncnc(Cl)c1NC1CCCCC1]

Prepared as described for N-cyclohexyl-4-iodopyridin-3-amine (Intermediate 17), to a stirred solution of 4-chloro-6-methoxypyrimidin-5-amine (CAS 15846-19-2; 0.15 g, 0.940 mmol) and cyclohexanone (CAS 108-94-1; 0.294 ml, 2.82 mmol) in anhydrous DCM (5 mL) under an atmosphere of nitrogen at 0° C. was added TiCl₄ solution (1M in DCM, 3.66 mL, 3.66 mmol). The reaction was stirred at room temperature for 2 h. Sodium triacetoxyborohydride (1.94 g, 9.15 mmol) was added portionwise and the reaction stirred at room temperature for 16 h. The crude product was purified by column chromatography (silica, 0-20% EtOAc/petroleum ether) to afford the title compound.

MS ES⁺: 242

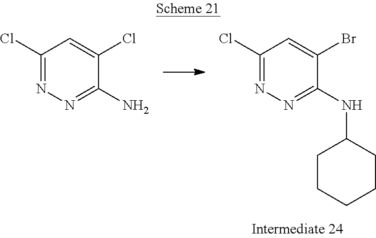 [SMILES: Nc1nnc(Cl)cc1Cl ; Clc1cc(Br)c(NC2CCCCC2)nn1]

### Intermediate 24 4-bromo-6-chloro-N-cyclohexylpyridazin-3-amine

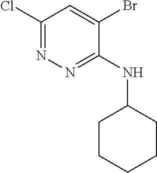 [SMILES: Clc1cc(Br)c(NC2CCCCC2)nn1]

To stirred solution of cyclohexanone (CAS 108-94-1; 1060 mg, 10.8 mmol) and 4-bromo-6-chloropyridazin-3-amine (CAS 446273-59-2; 750 mg, 3.60 mmol) in THF (10 mL) at 0° C. under N₂ was added titanium isopropoxide (IV) (1.16 mL, 3.96 mmol) dropwise. The reaction was allowed to stir at room temperature for 2 h and then cooled to 0° C. Sodium triacetoxyborohydride (4580 mg, 21.6 mmol) was added portionwise and then the reaction allowed to stir at room temperature. The reaction was poured into water and extracted with DCM. The organics were separated and concentrated.

The crude product was purified by column chromatography (silica, 0-100% EtOAc/petroleum ether) to afford the title compound. MS ES⁺: 292

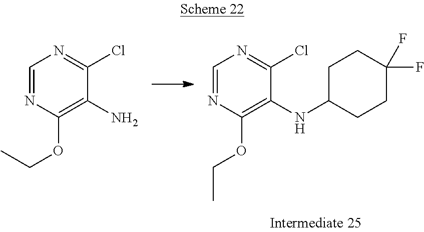 [SMILES: CCOc1ncnc(Cl)c1N ; CCOc1ncnc(Cl)c1NC1CCC(F)(F)CC1]

### Intermediate 25 4-chloro-N-(4,4-difluorocyclohexyl)-6-ethoxypyrimidin-5-amine

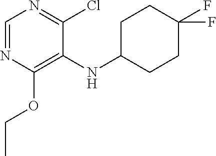 [SMILES: CCOc1ncnc(Cl)c1NC1CCC(F)(F)CC1]

Prepared as described for N-cyclohexyl-4-iodopyridin-3-amine (Intermediate 17), to stirred solution of 4,4-difluorocyclohexanone (CAS 22515-18-0; 1480 mg, 11.1 mmol) and 4-chloro-6-ethoxypyrimidin-5-amine (CAS 63291-59-8; 960 mg, 5.53 mmol) in DCM (15 mL) at 0° C. under N₂ was added TiCl₄ solution (1M in DCM, 6.08 mL, 6.08 mmol) dropwise. The reaction was allowed to stir at room temperature for 2 h and then cooled to 0° C. Sodium triacetoxyborohydride (2340 mg, 11.06 mmol) was added portionwise and then the reaction allowed to stir at room temperature overnight. The crude product was purified by column chromatography (silica, 0-50% EtOAc/petroleum ether) to afford the title compound.

¹H NMR (400 MHz, DMSO-d₆) δ ppm 1.33-1.41 (m, 3H) 1.51-1.64 (m, 2H) 1.77-1.89 (m, 4H) 2.00-2.09 (m, 2H) 3.66-3.81 (m, 1H) 4.39-4.47 (m, 3H) 8.08 (s, 1H) MS ES⁺: 292

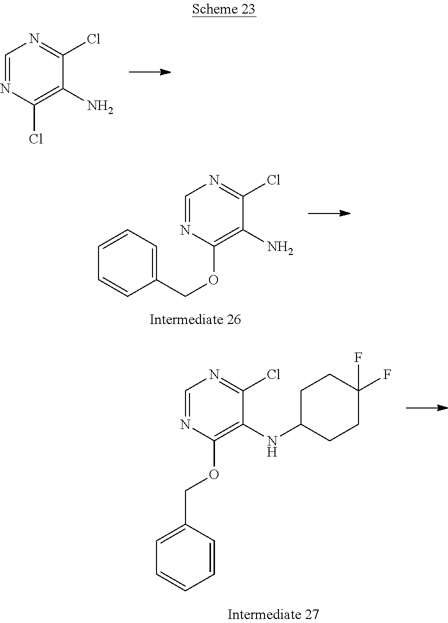 [SMILES: Nc1c(Cl)ncnc1Cl ; Nc1c(Cl)ncnc1OCc1ccccc1 ; FC1(F)CCC(Nc2c(Cl)ncnc2OCc2ccccc2)CC1]

### Intermediate 26 4-(benzyloxy)-6-chloropyrimidin-5-amine

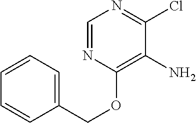 [SMILES: Nc1c(Cl)ncnc1OCc1ccccc1]

To a stirred solution of phenylmethanol (CAS 100-51-6; 791 mg, 7.32 mmol) in THF (10 mL) at 0° C. was added NaH, 60% dispersion in oil (0.305 g, 7.62 mmol) portionwise. The resulting suspension was allowed to stir for 15 minutes. 4,6-dichloropyrimidin-5-amine (CAS 5413-85-4; 1 g, 6.10 mmol) was then added slowly and the reaction allowed to warm to room temperature and stirred overnight. The reaction mixture was poured into water and extracted with DCM. The phases were separated and the organics concentrated in vacuo to afford the title compound.

¹H NMR (400 MHz, DMSO-d₆) δ ppm 5.45 (s, 2H) 5.49 (s, 2H) 7.31-7.36 (m, 1H) 7.38-7.44 (m, 2H) 7.47-7.52 (m, 2H) 7.92 (s, 1H) MS ES⁺: 236

### Intermediate 27 4-(benzyloxy)-6-chloro-N-(4,4-difluorocyclohexyl)pyrimidin-5-amine

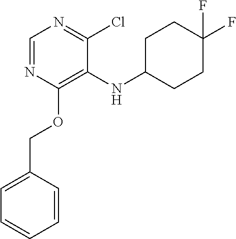 [SMILES: FC1(F)CCC(Nc2c(Cl)ncnc2OCc2ccccc2)CC1]

Prepared as described for N-cyclohexyl-4-iodopyridin-3-amine (Intermediate 17), to stirred solution of 4,4-difluorocyclohexanone (CAS 22515-18-0; 1.59 g, 11.9 mmol) and 4-(benzyloxy)-6-chloropyrimidin-5-amine (Intermediate 26; 1.4 g, 5.94 mmol) in DCM (15 mL) at 0° C. under N₂ was added TiCl₄ solution (1M in DCM, 6.53 mL, 6.53 mmol) dropwise. The reaction was allowed to stir at room temperature for 2 h and then cooled to 0° C. Sodium triacetoxyborohydride (2.52 g, 11.9 mmol) was added portionwise and then the reaction allowed to stir at room temperature overnight. The crude product was purified by column chromatography (silica, 0-100% EtOAc/petroleum ether) to afford the title compound.

¹H NMR (400 MHz, DMSO-d₆) δ ppm 1.74-1.82 (m, 2H) 1.89-1.98 (m, 2H) 2.26-2.39 (m, 2H) 2.40-2.46 (m, 2H) 3.64-3.78 (m, 1H) 4.47-4.53 (m, 1H) 5.47 (s, 2H) 7.30-7.46 (m, 3H) 7.46-7.54 (m, 2H) 8.12 (s, 1H)

MS ES⁺: 354

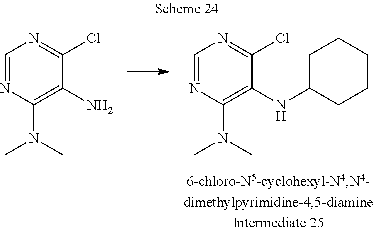 [SMILES: CN(C)c1ncnc(Cl)c1N ; CN(C)c1ncnc(Cl)c1NC1CCCCC1]

### Intermediate 28 6-chloro-5-N-cyclohexyl-4-N,4-N-dimethylpyrimidine-4,5-diamine

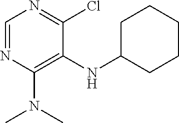 [SMILES: CN(C)c1ncnc(Cl)c1NC1CCCCC1]

Prepared as described for N-cyclohexyl-4-iodopyridin-3-amine (Intermediate 17), to a stirred solution of 6-chloro-N⁴,N⁴-dimethylpyrimidine-4,5-diamine (CAS 130623-81-3; 560 mg, 3.24 mmol) and cyclohexanone (CAS 108-94-1; 1.016 mL, 9.73 mmol) in anhydrous DCM (18 mL) under an atmosphere of N₂ at 0° C. was added dropwise TiCl₄ solution (1M in DCM, 3.66 mL, 3.66 mmol). The reaction was stirred at room temperature for 2 h. Sodium triacetoxyborohydride (1.94 g, 9.15 mmol) was added portionwise and the reaction stirred at room temperature for 16 h. The crude product was purified by column chromatography (silica, 0-50% EtOAc/petroleum ether) to afford the title compound.

MS ES⁺: 255

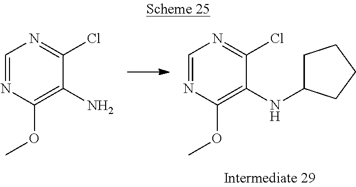

### Intermediate 29 4-chloro-N-cyclopentyl-6-methoxypyrimidin-5-amine

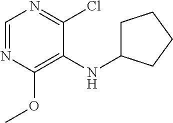

Prepared as described for N-cyclohexyl-4-iodopyridin-3-amine (Intermediate 17), to a stirred solution of 4-chloro-6-methoxypyrimidin-5-amine (CAS 15846-19-2; 200 mg, 1.25 mmol) and cyclopentanone (CAS 120-92-3; 0.33 mL, 3.76 mmol) in anhydrous DCM (6 mL) under an atmosphere of N₂ at 0° C. was added dropwise TiCl₄ solution (1M in DCM, 1.4 mL, 1.38 mmol). The reaction was stirred at room temperature for 2 h. Sodium triacetoxyborohydride (797 mg, 3.76 mmol) was added portionwise and the reaction stirred at room temperature for 16 h. The crude product was purified by column chromatography (silica, 0-50% EtOAc/petroleum ether) to afford the title compound.

¹H NMR (400 MHz, CHLOROFORM-d) δ ppm 1.33-1.53 (m, 2H) 1.55-1.82 (m, 4H) 1.83-2.00 (m, 2H) 3.73 (d, J=9 Hz, 1H) 4.04 (s, 3H) 4.18-4.42 (m, 1H) 8.08 (s, 1H) MS ES⁺: 228

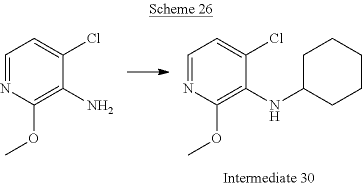

### Intermediate 30 4-chloro-N-cyclohexyl-2-methoxypyridin-3-amine

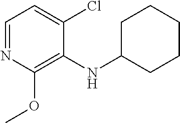 [SMILES: COc1nccc(Cl)c1NC1CCCCC1]

Prepared as described for N-cyclohexyl-4-iodopyridin-3-amine (Intermediate 17), to a stirred solution of 4-chloro-2-methoxypyridin-3-amine (CAS 934180-49-1; 250 mg, 1.58 mmol) and cyclohexanone (CAS 108-94-1; 309 mg, 3.15 mmol) in anhydrous DCM (10 mL) under an atmosphere of N₂ at 0° C. was added dropwise TiCl₄ solution (1M in DCM, 1.73 mL, 1.73 mmol). The reaction was stirred at room temperature for 2 h. Sodium triacetoxyborohydride (668 mg, 3.15 mmol) was added portionwise and the reaction stirred at room temperature overnight. The crude product was purified by column chromatography (silica, 0-100% EtOAc/petroleum ether) to afford the title compound.

¹H NMR (400 MHz, DMSO-d₆) δ ppm 1.46-1.71 (m, 6H) 1.75-2.01 (m, 4H) 3.54-3.64 (m, 1H) 3.89 (s, 3H) 4.02-4.08 (m, 1H) 6.97 (d, J 6 Hz, 1H) 7.54 (d, J=6 Hz, 1H)

MS ES⁺: 241

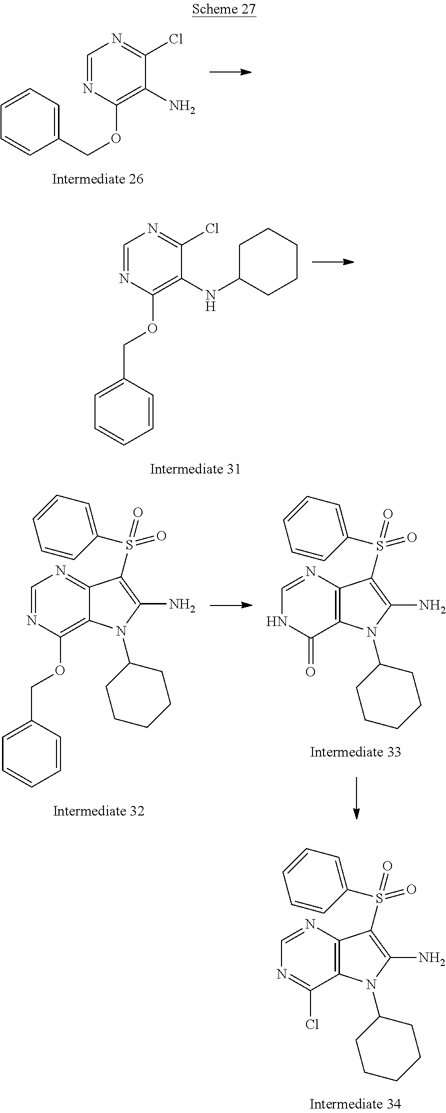 [SMILES: Nc1c(Cl)ncnc1OCc1ccccc1 ; Clc1ncnc(OCc2ccccc2)c1NC1CCCCC1 ; Nc1c(S(=O)(=O)c2ccccc2)c2ncnc(OCc3ccccc3)c2n1C1CCCCC1 ; Nc1c(S(=O)(=O)c2ccccc2)c2nc[nH]c(=O)c2n1C1CCCCC1 ; Nc1c(S(=O)(=O)c2ccccc2)c2ncnc(Cl)c2n1C1CCCCC1]

### Intermediate 31 4-(benzyloxy)-6-chloro-N-cyclohexylpyrimidin-5-amine

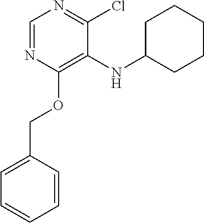 [SMILES: Clc1ncnc(OCc2ccccc2)c1NC1CCCCC1]

Prepared as described for N-cyclohexyl-4-iodopyridin-3-amine (Intermediate 17), to a mixture of cyclohexanone (CAS 108-94-1; 2.68 g, 27.3 mmol) and 4-(benzyloxy)-6-chloropyrimidin-5-amine (Intermediate 26; 3.22 g, 13.66 mmol) in DCM (50 mL) at 0° C. under N₂ was added dropwise TiCl₄ solution (1M in DCM, 15 mL, 15 mmol) dropwise. The reaction was allowed to stir at room temperature for 2 h and then cooled to 0° C. Sodium triacetoxyborohydride (5.79 g, 27.3 mmol) was added portionwise and then the reaction allowed to stir at room temperature overnight. The crude product was purified by column chromatography (silica, 0-100% EtOAc/petroleum ether) to afford the title compound.

MS ES⁺: 318

### Intermediate 32 7-(benzenesulfonyl)-4-(benzyloxy)-5-cyclohexyl-5H-pyrrolo[3,2-d]pyrimidin-6-amine

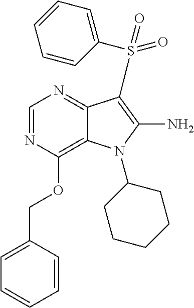 [SMILES: Nc1c(S(=O)(=O)c2ccccc2)c2ncnc(OCc3ccccc3)c2n1C1CCCCC1]

To a stirred solution of 2-(benzenesulfonyl)acetonitrile (CAS 7605-28-9; 1.96 g, 100.8 mmol) in DME (3 mL) at 0° C. was added NaH, 60% dispersion in oil (866 mg, 21.7 mmol). After to minutes the resulting suspension was added to a degassed solution of Pd(Ph₃P)₄ (313 mg, 0.27 mmol) and Pd(amphos)₂Cl₂ (192 mg, 0.271 mmol) in DME (2 mL). The resulting suspension was allowed to stir at room temperature for 20 minutes. 4-(benzyloxy)-6-chloro-N-cyclohexylpyrimidin-5-amine (Intermediate 31; 3.44 g, 10.8 mmol) was then added and the reaction mixture subjected to microwave irradiation at 120° C. for 2 h. The reaction mixture was poured into water and extracted with ethyl acetate. The organics were dried over MgSO₄ and concentrated. The crude product was purified by column chromatography (silica, 0-100% EtOAc/petroleum ether) to afford the title compound.

MS ES⁺: 346

### Intermediate 33 6-amino-5-cyclohexyl-7-(phenylsulfonyl)-3H-pyrrolo[3,2-d]pyrimidin-4(5H)-one

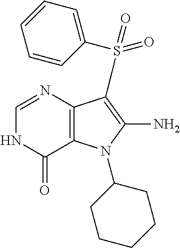 [SMILES: Nc1c(S(=O)(=O)c2ccccc2)c2nc[nH]c(=O)c2n1C1CCCCC1]

A suspension of 7-(benzenesulfonyl)-4-(benzyloxy)-5-cyclohexyl-5H-pyrrolo[3,2-d]pyrimidin-6-amine (Intermediate 32; 2.6 g, 5.62 mmol) and Pd/C (598 mg, 0.562 mmol) in MeOH (20 mL) was stirred under an atmosphere of hydrogen overnight. The reaction mixture was filtered through a pad of celite and the resulting filtrate concentrated. The crude product was purified by column chromatography (silica, o-10% MeOH/DCM) to afford the title compound.

¹H NMR (400 MHz, DMSO-d₆) δ ppm 1.33-1.96 (m, 9H) 2.41-2.55 (m, 2H) 7.47-7.62 (m, 4H) 7.63-7.70 (m, 2H) 7.83 (s, 1H) 8.04-8.11 (m, 2H)

MS ES⁺: 373

### Intermediate 34 7-(benzenesulfonyl)-4-chloro-5-cyclohexyl-5H-pyrrolo[3,2-d]pyrimidin-6-amine

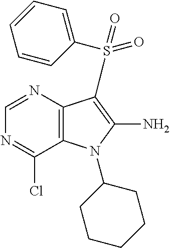 [SMILES: Nc1c(S(=O)(=O)c2ccccc2)c2ncnc(Cl)c2n1C1CCCCC1]

A solution of 6-amino-5-cyclohexyl-7-(phenylsulfonyl)-3H-pyrrolo[3,2-d]pyrimidin-4(5H)-one (Intermediate 33; 2.1 g, 5.64 mmol) in POCl₃ (8 mL, 86 mmol) was stirred at 80° C. overnight. The reaction mixture was allowed to cool and concentrated in vacuo. The crude residue was taken up in DCM and washed with water. The organics were separated and concentrated. The crude product was purified by column chromatography (silica, 0-100% EtOAc/petroleum ether) to afford the title compound.

¹H NMR (400 MHz, DMSO-d₆) δ ppm 1.33-1.44 (m, 3H) 1.58-1.65 (m, 1H) 1.76-1.91 (m, 4H) 2.25-2.38 (m, 2H) 4.83-4.99 (m, 1H) 7.51-7.68 (m, 5H) 8.04-8.11 (m, 2H) 8.42 (s, 1H)

MS ES⁺: 391

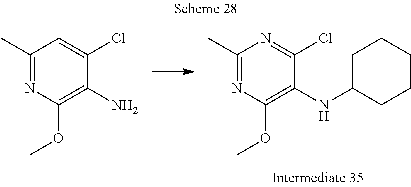 [SMILES: COc1nc(C)cc(Cl)c1N ; COc1nc(C)nc(Cl)c1NC1CCCCC1]

### Intermediate 35 4-chloro-N-cyclohexyl-6-methoxy-2-methylpyrimidin-5-amine

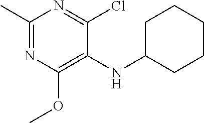 [SMILES: COc1nc(C)nc(Cl)c1NC1CCCCC1]

Prepared as described for N-cyclohexyl-4-iodopyridin-3-amine (Intermediate 17), to a stirred solution of cyclohexanone (CAS 108-94-1; 565 mg, 5.76 mmol) and 4-chloro-6-methoxy-2-methylpyrimidin-5-amine (CAS 88474-31-1; 500 mg, 2.88 mmol) in DCM (10 mL) at 0° C. under N₂ was added TiCl₄ solution (1M in DCM, 3.17 ml, 3.17 mmol) dropwise. The reaction was allowed to stir at room temperature for 2 h and then cooled to 0° C. Sodium triacetoxyborohydride (1.22 g, 5.76 mmol) was added portionwise and then the reaction allowed to stir at room temperature overnight. The crude product was purified by column chromatography (silica, 0-100% EtOAc/petroleum ether) to afford the title compound.

¹H NMR (400 MHz, DMSO-d₆) δ ppm 1.11-1.30 (m, 4H) 1.49-1.57 (m, 1H) 1.62-1.69 (m, 2H) 1.72-1.81 (m, 3H) 2.40 (s, 3H) 3.39-3.49 (m, 1H) 3.88-3.96 (m, 4H)

MS ES⁺: 256

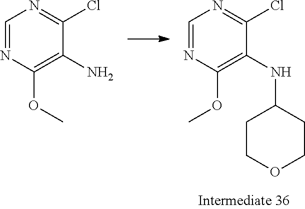

### Intermediate 36 4-chloro-6-methoxy-N-(tetrahydro-2H-pyran-4-yl)pyrimidin-5-amine

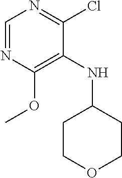 [SMILES: COc1ncnc(Cl)c1NC1CCOCC1]

Prepared as described for N-cyclohexyl-4-iodopyridin-3-amine (Intermediate 17), to a stirred solution of 4-chloro-6-methoxypyrimidin-5-amine (CAS 15846-19-2; 0.572 mL, 6.19 mmol) and oxan-4-one (CAS 29943-42-8; 0.33 mL, 3.76 mmol) in anhydrous DCM (6 mL) under an atmosphere of nitrogen at 0° C. was added dropwise TiCl₄ solution (1M in DCM, 3.41 mL, 3.41 mmol). The reaction was stirred at room temperature for 1 h. Sodium triacetoxyborohydride (1.31 g, 6.19 mmol) was added portionwise and the reaction stirred at room temperature over a weekend. The crude product was purified by column chromatography (silica, 50-100% EtOAc/petroleum ether) to afford the title compound.

¹H NMR (400 MHz, DMSO-d₆) δ ppm 1.40-1.53 (m, 2H) 1.69-1.77 (m, 2H) 3.26-3.35 (m, >2H due to overlap with water peak) 3.68-3.79 (m, 1H) 3.79-3.87 (m, 2H) 3.98 (s, 3H) 4.38 (d, J=10 Hz, 1H) 8.10 (s, 1H)

MS ES⁺: 244

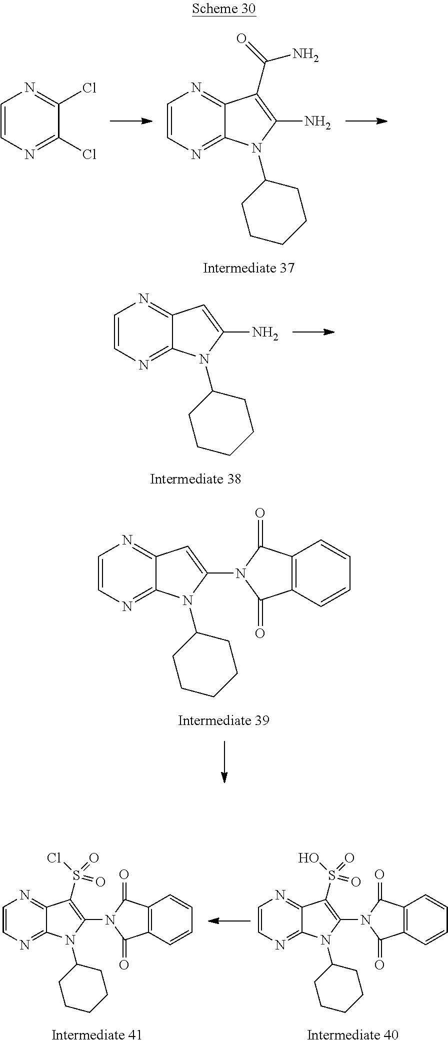 [SMILES: Clc1nccnc1Cl ; NC(=O)c1c(N)n(C2CCCCC2)c2nccnc12 ; Nc1cc2nccnc2n1C1CCCCC1 ; O=C1c2ccccc2C(=O)N1c1cc2nccnc2n1C1CCCCC1 ; O=C1c2ccccc2C(=O)N1c1c(S(=O)(=O)Cl)c2nccnc2n1C1CCCCC1 ; O=C1c2ccccc2C(=O)N1c1c(S(=O)(=O)O)c2nccnc2n1C1CCCCC1]

### Intermediate 37 6-amino-5-cyclohexyl-5H-pyrrolo[2,3-b]pyrazine-7-carboxamide

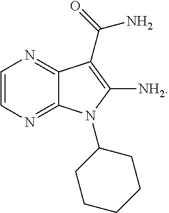 [SMILES: NC(=O)c1c(N)n(C2CCCCC2)c2nccnc12]

A mixture of 2,3-dichloropyrazine (CAS 4858-85-9; 10 g, 67.1 mmol), cesium carbonate (24 g, 73.8 mmol) and malononitrile (CAS 109-77-3; 4.88 g, 73.8 mmol) in DMSO (150 mL) was stirred at 125° C. for 90 minutes then allowed to cool to rt. Cyclohexanamine (CAS 108-91-8; 150 mL, 1.31 mol) was added and the reaction mixture was stirred at 130° C. for 4 days. After cooling to rt, 2M sodium hydroxide solution (200 mL; 0.4 mol) was added and the mixture was stirred at 115° C. for 24 hr. After cooling the mixture was diluted with water and extracted with EtOAc (×3). The combined organic extracts were washed with brine, dried over MgSO₄ and concentrated. The crude product was purified by column chromatography (silica, 0-100% EtOAc/petroleum ether) to afford the title compound.

¹H NMR (400 MHz, DMSO-d₆) δ ppm 1.20-1.33 (m, 1H) 1.35-1.47 (m, 2H) 1.64-1.78 (m, 3H) 1.81-1.89 (m, 2H) 2.37-2.49 (m, 2H) 4.32-4.44 (m, 1H) 7.08 (br. s., 1H) 7.42 (br. s., 1H) 7.77-7.89 (m, 3H) 8.04 (d, J=3 Hz, 1H)

MS ES⁺: 260.

### Intermediate 38 5-cyclohexyl-5H-pyrrolo[2,3-b]pyrazin-6-amine formate

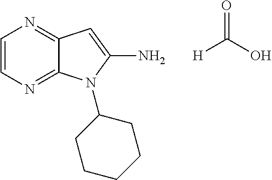 [SMILES: Nc1cc2nccnc2n1C1CCCCC1 ; O=CO]

A solution of 6-amino-5-cyclohexyl-5H-pyrrolo[2,3-b]pyrazine-7-carboxamide (Intermediate 37; 13.9 g, 53.6 mmol) in 50% aqueous sulfuric acid (100 mL) was heated at 100° C. for 2 h. The reaction mixture was allowed to cool to rt then poured into water and then basified to pH to with 2M NaOH. The resulting mixture was extracted with DCM (×3) and the organic extracts were concentrated in vacuo. The crude product was purified by column chromatography (C18-silica 5-95% methanol/water+0.1% formic acid) to afford the title compound.

¹H NMR (400 MHz, DMSO-d₆) δ ppm 1.25-1.46 (m, 3H) 1.64-1.73 (m, 3H) 1.80-1.89 (m, 2H) 2.42-2.54 (m, 2H) 4.21-4.32 (m, 1H) 5.34 (s, 1H) 6.48 (br. s., 2H) 7.61 (d, J=3 Hz, 1H) 7.86 (d, J=3 Hz, 1H) 8.16 (s, 1H)

MS ES⁺: 217

### Intermediate 3 2-{5-cyclohexyl-5-pyrrolo[2,3-b]pyrazin-6-yl}-2,3-dihydro-1H-isoindole-1,3-dione

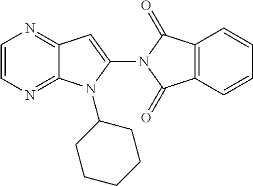 [SMILES: O=C1c2ccccc2C(=O)N1c1cc2nccnc2n1C1CCCCC1]

A solution of 5-cyclohexyl-5H-pyrrolo[2,3-b]pyrazin-6-amine formate (Intermediate 38; 5 g, 19.1 mmol) in DCM (0 mL) was treated with triethylamine (12.9 mL, 92 mmol) followed by phthaloyl dichloride (CAS 88-95-9; 4.93 g, 24.3 mmol). The reaction mixture was allowed to stir at rt for 3 hours then poured into water and extracted with DCM. The organic phase was separated and concentrated to yield the title compound, which was used without further purification.

¹H NMR (400 MHz, DMSO-d₆) δ ppm 1.00-1.09 (m, 2H) 1.16-1.41 (m, 3H) 1.58-1.65 (m, 1H) 1.73-1.86 (m, 4H) 4.22-4.32 (m, 1H) 6.84 (s, 1H) 7.96-8.02 (m, 2H) 8.04-8.10 (m, 2H) 8.37-8.41 (m, 1H) 8.48-8.54 (m, 1H)

### Intermediate 40 5-cyclohexyl-6-(1,3-dioxo-2,3-dihydro-1H-isoindol-2-yl)-5H-pyrrolo[2,3-b]pyrazine-7-sulfonic add

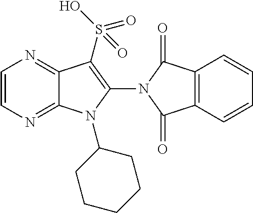 [SMILES: O=C1c2ccccc2C(=O)N1c1c(S(=O)(=O)O)c2nccnc2n1C1CCCCC1]

A solution of 2-{5-cyclohexyl-5H-pyrrolo[2,3-b]pyrazin-6-yl}-2,3-dihydro-1H-isoindole-1,3-dione (Intermediate 39; 8.63 g, 24.9 mmol) and acetic anhydride (23.5 mL, 249 mmol) in dichloromethane (100 mL) was cooled to 0° C. then sulfuric acid (6.64 mL, 125 mmol) was added dropwise. After 2 h the reaction mixture was diluted with water and extracted with DCM. The organic phase was concentrated and then azeotroped with toluene to yield the title compound.

MS ES⁺: 427.

### Intermediate 41 5-cyclohexyl-6-(1,3-dioxo-2,3-dihydro-1H-isoindol-2-yl)-5H-pyrrolo[2,3-b]pyrazine-7-sulfonyl chloride

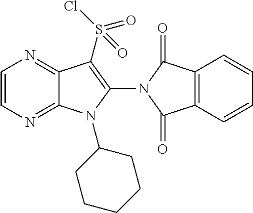 [SMILES: O=C1c2ccccc2C(=O)N1c1c(S(=O)(=O)Cl)c2nccnc2n1C1CCCCC1]

A solution of 5-cyclohexyl-6-(1,3-dioxo-2,3-dihydro-1H-isoindol-2-yl)-5H-pyrrolo[2,3-b]pyrazine-7-sulfonic acid (Intermediate 40; 10.63 g, 24.9 mmol) in phosphorus oxychloride (50 mL, 536 mmol) was treated with phosphorus pentachloride (5.42 g, 26.0 mmol) and heated to 80° C. for 1.5 h. The reaction mixture was slowly quenched into warm water. The aqueous mixture was allowed to cool to rt and extracted with DCM. The organic phase was concentrated to yield the title compound.

¹H NMR (400 MHz, DMSO-d₆) δ ppm 1.18-1.49 (m, 3H) 1.63-1.68 (m, 1H) 1.75-1.93 (m, 4H) 2.53-2.64 (m, 2H) 4.81 (s, 1H) 8.04-8.09 (m, 2H) 8.13-8.19 (m, 2H) 8.78 (d, J=2.27 Hz, 1H) 8.90 (d, J=2.53 Hz, 1H)

MS ES⁺: 445

### Intermediate 42 5-cyclohexyl-6-(1,3-dioxo-2,3-dihydro-1H-isoindol-2-yl)-N-phenyl-5H-pyrrolo[2,3-b]pyrazine-7-sulfonamide

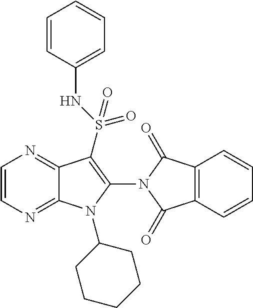 [SMILES: O=C1c2ccccc2C(=O)N1c1c(S(=O)(=O)Nc2ccccc2)c2nccnc2n1C1CCCCC1]

To a stirred solution of 5-cyclohexyl-6-(1,3-dioxo-2,3-dihydro-1H-isoindol-2-yl)-5H-pyrrolo[2,3-b]pyrazine-7-sulfonyl chloride (Intermediate 41; 100 mg, 0.225 mmol) in THF (1 mL) was added DMAP (28 mg, 0.225 mmol) and aniline (CAS 62-53-3; 42 mg, 0.450 mmol) and the reaction mixture allowed to stir at room temperature overnight. The reaction mixture was diluted with water and extracted with DCM. The organics were separated and concentrated. The crude material was purified by column chromatography (silica, 0-50% EtOAc/petroleum ether) to afford the title compound. ¹H NMR (40 MHz, DMSO-d₆) δ ppm 1.19-1.28 (m, 1H) 1.30-1.47 (m, 2H) 1.56-1.69 (m, 1H) 1.72-1.81 (m, 3H) 2.40-2.48 (m, 3H) 4.52-4.65 (m, 1H) 6.84-6.93 (m, 1H) 6.96-6.70 (m, 2H) 7.06-7.12 (m, 2H) 8.01-8.10 (m, 2H) 8.11-8.17 (m, 2H) 8.57 (d, J=3 Hz, 1H) 8.69 (d, J=3 Hz, 1H) 10.67 (s, 1H)

MS ES⁺: 502

### Intermediate 43 5-cyclohexyl-6-(1,3-dioxo-2,3-dihydro-1H-isoindol-2-yl)-N-(pyridin-3-yl)-5H-pyrrolo[2,3-b]pyrazine-7-sulfonamide

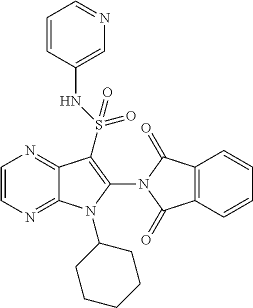 [SMILES: O=C1c2ccccc2C(=O)N1c1c(S(=O)(=O)Nc2cccnc2)c2nccnc2n1C1CCCCC1]

To a stirred solution of 5-cyclohexyl-6-(1,3-dioxo-2,3-dihydro-1H-isoindol-2-yl)-5H-pyrrolo[2,3-b]pyrazine-7-sulfonyl chloride (Intermediate 41; 1 mg, 0.225 mmol) in THF (1 mL) was added DMAP (28 mg, 0.225 mmol) and pyridin-3-amine (CAS 462-08-8; 42 mg, 0.450 mmol). The reaction mixture was allowed to stir at room temperature overnight. The reaction mixture was diluted with water and extracted with DCM. The crude material was purified by column chromatography (silica, 0-100% EtOAc/petroleum ether) to afford the title compound.

MS ES⁺: 503

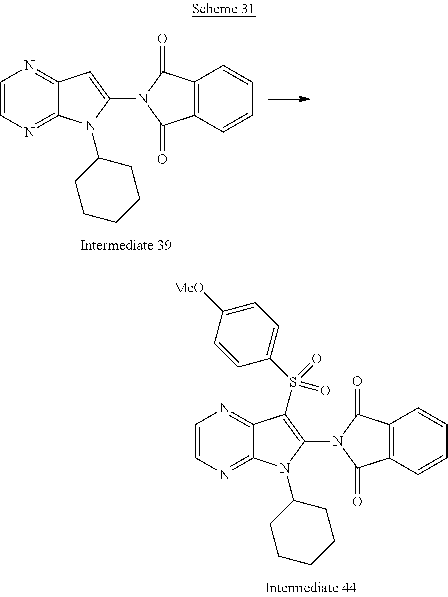 [SMILES: O=C1c2ccccc2C(=O)N1c1cc2nccnc2n1C1CCCCC1 ; COc1ccc(S(=O)(=O)c2c(N3C(=O)c4ccccc4C3=O)n(C3CCCCC3)c3nccnc23)cc1]

### Intermediate 44 2-(5-cyclohexyl-7-((4-methoxyphenyl)sulfonyl)-5H-pyrrolo[2,3-b]pyrazin-6-yl)isoindoline-1,3-dione

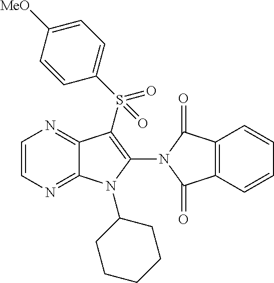 [SMILES: COc1ccc(S(=O)(=O)c2c(N3C(=O)c4ccccc4C3=O)n(C3CCCCC3)c3nccnc23)cc1]

A mixture of silver trifluoromethanesulfonate (45 mg, 0.173 mmol), 4-methoxybenzene-1-sulfonyl chloride (36 mg, 0.173 mmol) and 2-{5-cyclohexyl-5H-pyrrolo[2,3-b]pyrazin-6-yl}-2,3-dihydro-1H-isoindole-1,3-dione (Intermediate 39; 30 mg, 0.087 mmol) in nitrobenzene (0.5 mL) was subjected to microwave heating to 120° C. for 40 minutes. The reaction mixture was partitioned between water and DCM then the organic phase was concentrated in vacuo and the residue was purified by column chromatography on silica (silica, 5-50% EtOAc/petroleum ether) to afford the title compound.

¹H NMR (4 MHz, DMSO-d₆) δ ppm 1.22-1.32 (m, 2H) 1.32-1.46 (m, 2H) 1.60-1.67 (m, 1H) 1.70-1.85 (m, 5H) 3.81 (s, 3H) 4.63-4.74 (m, 1H) 7.08-7.16 (m, 2H) 7.89-7.96 (m, 1H) 8.04-8.10 (m, 1H) 8.14-8.21 (m, 2H) 8.60 (d, J=2 Hz, 1H) 8.72 (d, J=2 Hz, 1H)

MS ES⁺: 517.

Intermediates 45 to 54 were prepared by analogous methods and the data are given in Table 1. Where reactions failed to proceed to completion, further sulfonyl chloride was added and the temperature was increased (up to 150° C.) as required. Conventional heating in a sealed tube could also be employed.

TABLE 1

| | | | | Column | |
|---|---|---|---|---|---|
| | | | | chromatography | MS |
| Intermediate | Name of compound | Structure | Sulfonyl chloride | gradient | ES+ |
| 45 | 2-(5-cyclohexyl-7- (cyclopropylsulfonyl)-5H- pyrrolo[2,3-b]pyrazin-6- yl)isoindoline-1,3-dione | 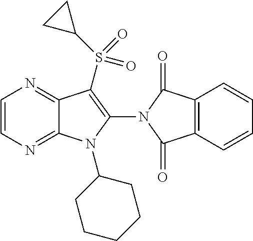 [SMILES: O=C1c2ccccc2C(=O)N1c1c(S(=O)(=O)C2CC2)c2nccnc2n1C1CCCCC1] | Cyclopropanesulfonyl chloride (CAS 139631-62-2) | 5-50% EtOAc/petrol | 451 |
| 46 | 2-(5-cyclohexyl-7-((3- fluorophenyl)sulfonyl)- 5H-pyrrolo[2,3-b]pyrazin- 6-yl)isoindoline-1,3-dione | 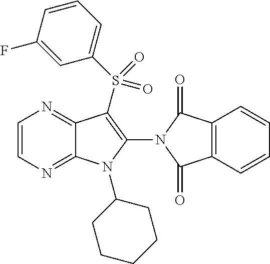 [SMILES: O=C1c2ccccc2C(=O)N1c1c(S(=O)(=O)c2cccc(F)c2)c2nccnc2n1C1CCCCC1] | 3-fluorobenzene-1- sulfonyl chloride (CAS 701-27-9) | 5-50% EtOAc/petrol | 505 |
| 47 | 2-(5-cyclohexyl-7-((2- fluorophenyl)sulfonyl)- 5H-pyrrolo[2,3-b]pyrazin- 6-yl)isoindoline-1,3-dione | 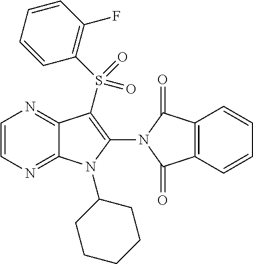 [SMILES: O=C1c2ccccc2C(=O)N1c1c(S(=O)(=O)c2ccccc2F)c2nccnc2n1C1CCCCC1] | 2-fluorobenzene-1- sulfonyl chloride (CAS 2905-21-7) | 5-50% EtOAc/petrol | 505 |
| 48 | 2-(5-cyclohexyl-7-((3- methoxyphenyl)sulfonyl)- 5H-pyrrolo[2,3-b]pyrazin- 6-yl)isoindoline-1,3-dione | 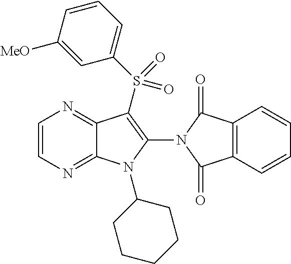 [SMILES: COc1cccc(S(=O)(=O)c2c(N3C(=O)c4ccccc4C3=O)n(C3CCCCC3)c3nccnc23)c1] | 3-methoxybenzene-1- sulfonyl chloride (CAS 10130-74-2) | 5-50% EtOAc/petrol | 517 |
| 49 | 4-((5-cyclohexyl-6-(1,3- dioxoisoindolin-2-yl)-5H- pyrrolo[2,3-b]pyrazin-7- yl)sulfonyl)benzonitrile | 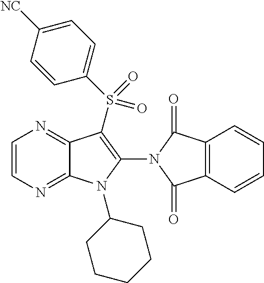 [SMILES: N#Cc1ccc(S(=O)(=O)c2c(N3C(=O)c4ccccc4C3=O)n(C3CCCCC3)c3nccnc23)cc1] | 4-cyanobenzene-1- sulfonyl chloride (CAS 49584-26-1) | 5-50% EtOAc/petrol | 512 |
| 50 | 2-(7-((3-chloro-4- methoxyphenyl)sulfonyl)- 5-cyclohexyl-5H- pyrrolo[2,3-b]pyrazin-6- yl)isoindoline-1,3-dione | 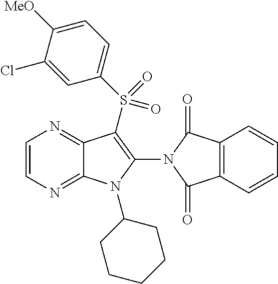 [SMILES: COc1ccc(S(=O)(=O)c2c(N3C(=O)c4ccccc4C3=O)n(C3CCCCC3)c3nccnc23)cc1Cl] | 3-chloro-4- methoxybenzene-1- sulfonyl chloride (CAS 22952-43-8) | 5-70% EtOAc/petrol | 551 |
| 51 | 2-(5-cyclohexyl-7-((6- methoxypridin-3- yl)sulfonyl)-5H- pyrrolo[2,3-b]pyrazin-6- yl)isoindoline-1,3-dione | 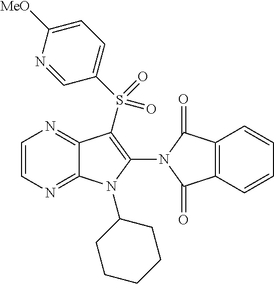 [SMILES: COc1ccc(S(=O)(=O)c2c(N3C(=O)c4ccccc4C3=O)n(C3CCCCC3)c3nccnc23)cn1] | 6-methoxypyridine-3- sulfonyl chloride (CAS 312300-42-8) | 5-75% EtOAc/petrol | 518 |
| 52 | 2-(5-cyclohexyl-7-((4- (trifluoromethoxy)phenyl) sulfonyl)-5H-pyrrolo[2,3- b]pyrazin-6- yl)isoindoline-1,3-dione | 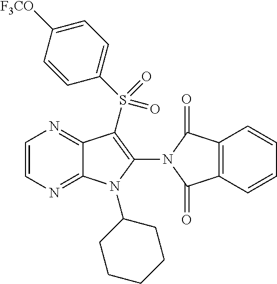 [SMILES: O=C1c2ccccc2C(=O)N1c1c(S(=O)(=O)c2ccc(OC(F)(F)F)cc2)c2nccnc2n1C1CCCCC1] | 4-(trifluoromethoxy) benzenesulfonyl chloride (CAS 94108-56-2) | 5-80% EtOAc/petrol | 571 |
| 53 | 2-(5-cyclohexyl-7-((2,3- ((2,3-dihydrobenzo[b][1,4] dioxin-6-yl)sulfonyl)-5H- pyrrolo[2,3-b]pyrazin-6- yl)isoindoline-1,3-dione | 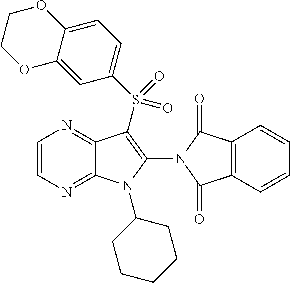 [SMILES: O=C1c2ccccc2C(=O)N1c1c(S(=O)(=O)c2ccc3c(c2)OCCO3)c2nccnc2n1C1CCCCC1] | 2,3-dihydrobenzo[b] [1,4]dioxine-6- sulfonyl chloride (CAS 63758-12-3) | 5-40% EtOAc/petrol | 545 |
| 54 | 2-(5-cyclohexyl-7-((4- (difluoromethoxy)phenyl) sulfonyl)-5H-pyrrolo[2,3- b]pyrazin-6- yl)isoindoline-1,3-dione | 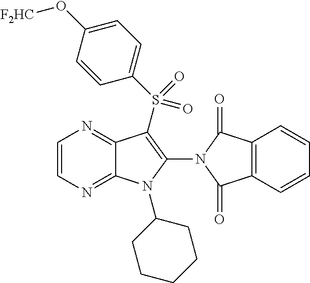 [SMILES: O=C1c2ccccc2C(=O)N1c1c(S(=O)(=O)c2ccc(OC(F)F)cc2)c2nccnc2n1C1CCCCC1] | 4-(difluoromethoxy) benzene-1-sulfonyl chloride (CAS 351003-34-4) | 5-40% EtOAc/petrol | 553 |

### 2. Final Compounds

### Example 1 7-(benzenesulfonyl)-5-cyclohexyl-5H-pyrrolo[2,3-b]pyrazin-6-amine

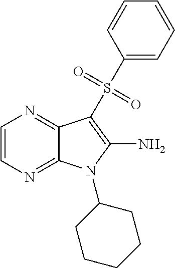 [SMILES: Nc1c(S(=O)(=O)c2ccccc2)c2nccnc2n1C1CCCCC1]

To a stirred solution of 2-(benzenesulfonyl)-2-(3-chloropyrazin-2-yl)acetonitrile (Intermediate 1; 50 g, 170 mmol) and cyclohexanamine (CAS 108-91-8; 97 mL, 850-mmol) in DMSO (100 mL) was added triethylamine (26 mL, 190 mmol). The reaction was heated thermally at 170° C. for 48 h. More cyclohexanamine (97 mL, 850 mmol) and triethylamine (26 mL, 190 mmol) were added and the reaction heated thermally at 185° C. for 24 h. The reaction was allowed to cool and diluted with brine. The resulting mixture was extracted with ethyl acetate and the organics washed with water and then with water/brine (1:1). The organics were dried (MgSO₄) and concentrated in vacuo. The crude product was loaded onto a plug of silica (10 g) and eluted using 0-50% EtOAc/petroleum ether. Product fractions were concentrated and this purification process repeated another 3 times. The product fractions were concentrated. The resulting residue was recrystallised from hot ethanol to afford the title compound.

¹H NMR (40 MHz, DMSO-d₆) δ ppm 1.20-1.29 (m, 1H) 1.33-1.48 (m, 2H) 1.62-1.76 (m, 3H) 1.77-1.88 (m, 2H) 2.39-2.48 (m, 2H) 4.33-4.47 (m, 1H) 7.52-7.64 (m, 5H) 7.86-7.91 (m, 1H) 8.01-8.07 (m, 2H) 8.07-8.12 (m, 1H)

MS ES⁺: 357

### Example 2 5-cycloheptyl-7-[(4-methylbenzene)sulfonyl]-5H-pyrrolo[2,3-b]pyrazin-6-amine

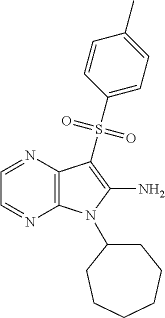 [SMILES: Cc1ccc(S(=O)(=O)c2c(N)n(C3CCCCCC3)c3nccnc23)cc1]

A neat mixture of 2-(3-chloropyrazin-2-yl)-2-(4-methylbenzenesulfonyl)acetonitrile (Intermediate 2; 109 mg, 0.35 mmol) and cycloheptanamine (CAS 5452-35-7; 1.13 mL, 8.85 mmol) was heated in a microwave at 170° C. for 1 h and 45 mins. The reaction mixture was evaporated and purified by column chromatography (preparative HPLC, 40-80% acetonitrile/water (with 0.1% ammonia)) to afford the title compound.

¹H NMR (400 MHz, DMSO-d₆) δ ppm 1.40-1.75 (m, 8H), 2.32 (s, 3H), 2.35-2.47 (m, 2H) 2.95-3.07 (m, 2H) 4.45-4.60 (br. m., 1H) 7.33 (d, J=8 Hz, 2H) 7.54 (br. s., 2H) 7.82 (d, J=3 Hz, 1H) 7.91 (d, J=8 Hz, 2H) 8, (d, J=3 Hz, 1H)

MS ES⁺: 385

### Example 3 5-cycloheptyl-7-[(4-methylbenzene)sulfonyl]-5H-pyrrolo[2,3-b]pyrazin-6-amine

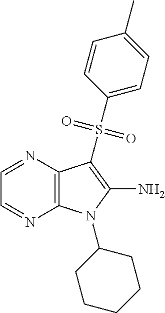 [SMILES: Cc1ccc(S(=O)(=O)c2c(N)n(C3CCCCC3)c3nccnc23)cc1]

Prepared as described for 5-cycloheptyl-7-[(4-methylbenzene)sulfonyl]-5H-pyrrolo[2,3-b]pyrazin-6-amine (Example 2), a neat mixture of 2-(3-chloropyrazin-2-yl)-2-(4-methylbenzenesulfonyl)acetonitrile (Intermediate 2; 109 mg, 0.35 mmol) and cyclohexanamine (CAS 108-91-8; 1.01 mL, 8.85 mmol) was heated in a microwave at 170° C. for 1 h and 45 mins. The reaction mixture was evaporated and purified by column chromatography (preparative HPLC, 30-70% acetonitrile/water (with 0.1% ammonia)) to afford the title compound.

¹H NMR (400 MHz, DMSO-d₆) δ ppm 1.15-1.31 (m, 1H) 1.32-1.48 (m, 2H) 1.60-1.76 (m, 3H) 1.77-1.87 (m, 2H) 2.33 (s, 3H) 2.37-2.48 (m, 2H) 4.32-4.44 (m, 1H) 7.35 (d, J=8 Hz, 2H) 7.57 (s, 2H) 7.88 (d, J=3 Hz, 1H) 7.92 (d, J=8 Hz, 2H) 8.08 (d, J=3 Hz, 1H)

MS ES⁺: 371

### Example 4 5-cyclopentyl-7-[(4-methylbenzene)sulfonyl]-5H-pyrrolo[2,3-b]pyrazin-6-amine

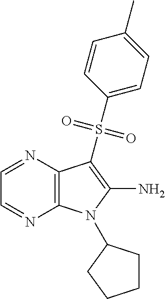 [SMILES: Cc1ccc(S(=O)(=O)c2c(N)n(C3CCCC3)c3nccnc23)cc1]

Prepared as described for 5-cycloheptyl-7-[(4-methylbenzene)sulfonyl]-5H-pyrrolo[2,3-b]pyrazin-6-amine (Example 2), a neat mixture of 2-(3-chloropyrazin-2-yl)-2-(4-methylbenzenesulfonyl)acetonitrile (Intermediate 2; 109 mg, 0.35 mmol) and cyclopentanamine (CAS 1003-03-8; 0.873 mL, 8.85 mmol) was heated in a microwave at 170° C. for 1 h and 45 mins. The reaction mixture was evaporated and purified by column chromatography (preparative HPLC, 30-70% acetonitrile/water (with 0.1% ammonia)) to afford the title compound.

¹H NMR (400 MHz, CHLOROFORM-d) δ ppm 1.69-1.85 (m, 2H) 1.96-2.16 (m, 4H) 2.21-2.35 (m, 2H) 2.40 (s, 3H) 4.80-4.92 (m, 1H) 6.08 (br. &, 2H) 7.27-7.33 (m, 2H) 7.92 (d, J=3 Hz, 1H) 8.10 (d, J=8 Hz, 2H) 8.26 (d, J=3 Hz, 1H)

MS ES⁺: 357

### Example 5 7-[(4-chlorobenzene)sulfonyl]-5-cyclohexyl-5H-pyrrolo[2,3-b]pyrazin-6-amine

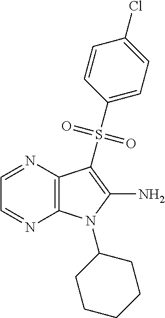 [SMILES: Nc1c(S(=O)(=O)c2ccc(Cl)cc2)c2nccnc2n1C1CCCCC1]

A stirred solution of 2-(4-chlorophenylsulfonyl)-2-(3-chloropyrazin-2-yl)acetonitrile (Intermediate 3; 218 mg 0.664 mmol) and cyclohexanamine (CAS 108-91-8; 228 μL, 1.99 mmol) in N-methyl-2-pyrrolidinone (1.3 mL) was heated in a microwave at 170° C. for 2 h. More cyclohexanamine (228 μL, 1.99 mmol) was then added and the reaction was heated in a microwave at 170° C. for 2 h. The reaction mixture was diluted with EtOAc, washed with brine and water, dried (H frit) and evaporated to dryness. The crude product was purified by column chromatography (silica, 0-30% EtOAc/petroleum ether) to afford the title compound.

¹H NMR (400 MHz, CHLOROFORM-d) δ ppm 1.24-1.54 (m, 3H) 1.74-1.83 (m, 1H) 1.84-1.93 (m, 2H) 1.93-2.01 (m, 2H) 2.29-2.46 (m, 2H) 4.17-4.33 (m, 1H) 6.14 (br. s., 2H) 7.46 (d, J=9 Hz, 2H) 7.95 (d, J=3 Hz, 1H) 8.16 (d, J=9 Hz, 2H) 8.25 (d, J=3 Hz, 1H)

MS ES⁺: 391

### Example 6 5-cyclohexyl-7-[(4-fluorobenzene)sulfonyl]-5H-pyrrolo[2,3-b]pyrazin-6-amine

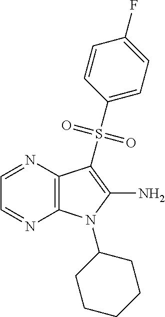 [SMILES: Nc1c(S(=O)(=O)c2ccc(F)cc2)c2nccnc2n1C1CCCCC1]

A stirred solution of 2-(3-chloropyrazin-2-yl)-2-(4-fluorophenylsulfonyl)acetonitrile (Intermediate 4; 101 mg, 0.324 mmol) and cyclohexanamine (CAS 108-91-8; 111 μL, 0.972 mmol) in N-methyl-2-pyrrolidinone (650 μL) was heated in a microwave at 170° C. for 2 h. More cyclohexanamine (200 μL, 1.75 mmol) was added and the reaction heated in a microwave at 170° C. for 2 h. The reaction mixture was diluted with EtOAc, washed with brine and water, dried (H fit) and evaporated to dryness. The crude product was purified by column chromatography (silica, 0-30% EtOAc/petroleum ether) to afford the title compound.

¹H NMR (400 MHz, METHANOL-d₄) δ ppm 1.26-1.57 (m, 3H) 1.69-1.83 (m, 3H) 1.86-1.98 (m, 2H) 2.48-2.64 (m, 2H) 4.25-4.38 (m, 1H) 7.19-7.27 (m, 2H) 7.90 (d, J=3 Hz, 1H) 8.03 (d, J=3 Hz, 1H) 8.10-8.18 (m, 2H)

MS ES⁺: 375

### Example 7 5-cyclohexyl-7-{[4-(propan-2-yloxy)benzene]sulfonyl}-5H-pyrrolo[2,3-b]pyrazin-6-amine

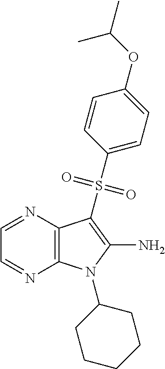 [SMILES: CC(C)Oc1ccc(S(=O)(=O)c2c(N)n(C3CCCCC3)c3nccnc23)cc1]

A stirred solution of 2-(3-chloropyrazin-2-yl)-2-(4-isopropoxyphenylsulfonyl)acetonitrile (Intermediate 5; 204 mg, 0.580 mmol) and cyclohexanamine (CAS 108-91-8; 199 μL, 1.74 mmol) in N-methyl-2-pyrrolidinone (1.1 mL) was heated in a microwave at 170° C. for 2 h. More cyclohexanamine (200 μL, 1.75 mmol) was added and the reaction heated in a microwave at 170° C. for 2 h. The reaction mixture was diluted with EtOAc, washed with brine and water, dried (H frit) and evaporated to dryness. The crude product was purified by column chromatography (silica, 0-50% EtOAc/petroleum ether) to afford the title compound.

¹H NMR (400 MHz, DMSO-d₆) δ ppm 1.25 (d, J=6 Hz, 6H) 1.32-1.48 (m, 2H) 1.62-1.76 (m, 3H) 1.77-1.87 (m, 2H) 2.36-2.49 (m, 3H) 4.32-4.44 (m, 1H) 4.62-4.73 (m, 1H) 7.03 (d, J=9 Hz, 2H) 7.54 (br. s, 2H) 7.88 (d, J=3 Hz, 1H) 7.94 (d, J=9 Hz, 2H) 8.08 (d, J=3 Hz, 1H)

MS ES⁺: 415

### Example 8 5-cyclohexyl-7-(thiophene-2-sulfonyl)-5H-pyrrolo[2,3-b]pyrazin-6-amine

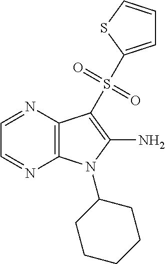 [SMILES: Nc1c(S(=O)(=O)c2cccs2)c2nccnc2n1C1CCCCC1]

A stirred solution of 2-(3-chloropyrazin-2-yl)-2-(thiophen-2-ylsulfonyl)acetonitrile (Intermediate 6; 74 mg, 0.247 mmol) and cyclohexanamine (CAS 108-91-8; 282 μl, 2.47 mmol) in DMSO (120 μL) was heated in a microwave at 170° C. for 2.5 h. The reaction mixture was diluted with DMSO and purified by column chromatography (preparative HPLC, 30-70% acetonitrile/water (with 0.1% ammonia)) to afford the title compound.

¹H NMR (4 MHz, METHANOL-d₄) δ ppm 1.29-1.59 (m, 3H) 1.71-1.86 (m, 3H) 1.90-1.99 (m, 2H) 2.52-2.67 (m, 2H) 4.28-4.40 (m, 1H) 7.06-7.11 (m, 1H) 7.68-7.73 (m, 1H) 7.81-7.85 (m, 1H) 7.93 (d, J=3 Hz, 1H) 8.06 (d, J=3 Hz, 1H)

MS ES⁺: 363

### Example 9 3-(benzenesulfonyl)-1-cyclohexyl-1H-[3,2-b]pyridin-2-amine

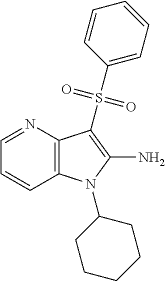 [SMILES: Nc1c(S(=O)(=O)c2ccccc2)c2ncccc2n1C1CCCCC1]

To a stirred solution of solution of 2-(benzenesulfonyl)acetonitrile (CAS 7605-28-9; 555 mg, 3.07 mmol) in DME (3 mL) at 0° C. under a flow of N₂ was added sodium hydride (60% dispersion in oil, 223 mg, 5.57 mmol) and the resulting suspension allowed to stir for to minutes. In a separate flask Pd(Ph₃P)₄ (CAS 014221-01-3; 161 mg, 0.139 mmol) in DME (3 mL) was degassed with N₂. The suspension of pre-formed sodium salt of 2-(benzenesulfonyl)acetonitrile was added to the second vessel. After stirring for a further 10 minutes 2-bromo-N-cyclohexylpyridin-3-amine (Intermediate 7; 711 mg, 2.79 mmol) was added and the reaction mixture subjected to microwave irradiation at 120° C. for 1.5 h. The reaction mixture was poured into water and extracted with ethyl acetate and then the organics washed with brine. The organics were dried over MgSO₄ and concentrated in vacuo. The crude product was purified by column chromatography (silica, 0-500% EtOAc/DCM) to afford crude product. The crude product was triturated with hot IPA and then filtered and dried to afford the title compound.

¹H NMR (400 MHz, DICHLOROMETHANE-d₂) δ ppm 1.13-1.37 (m, 1H) 1.38-1.54 (m, 2H) 1.73-1.85 (m, 1H) 1.86-2.16 (m, 6H) 3.91-4.04 (m, 1H) 5.88 (br. s., 2H) 6.89-6.98 (m, 1H) 7.40-7.59 (m, 4H) 8.13-8.20 (m, 2H) 8.22-8.30 (m, 1H)

MS ES⁺: 356

### Example 10 1-cyclopentyl-3-[(4-methylbenzene)sulfonyl]-1H-pyrrolo[3,2-b]pyridin-2-amine

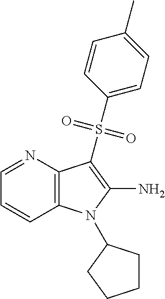 [SMILES: Cc1ccc(S(=O)(=O)c2c(N)n(C3CCCC3)c3cccnc23)cc1]

To a stirred solution of 3-(4-methylbenzenesulfonyl)-1H-pyrrolo[3,2-b]pyridin-2-amine (Intermediate 10; 250 mg, 0.7 mmol) in DMF (10 mL) was added DBU (264 mg, 1.4 mmol) and cyclopentyl bromide (194 mg, 1.0 mmol). The reaction was heated in a sealed tube at 80° C. The reaction mixture was poured into water and extracted with ethyl acetate. The organics were dried over Na₂SO₄ and concentrated in vacuo. The crude product was purified by column chromatography (preparative HPLC, 5-95% acetonitrile/water (with 0.1% ammonia)) to afford the title compound.

¹H NMR (400 MHz, DMSO-d₆) δ ppm 1.61-1.71 (m, 2H) 1.90-2.02 (m, 6H) 2.32 (s, 3H) 4.84-4.92 (m, 1H) 6.87-6.94 (m, 1H) 7.13 (s, 2H) 7.33 (d, J=8 Hz, 2H) 7.48-7.55 (m, 1H) 7.95 (d, J=8 Hz, 2H) 8.11-8.18 (m, 1H)

MS ES⁺: 356

### Example 11 1-cyclohexyl-3-[(4-methylbenzene)sulfonyl]-1H-pyrrolo[2,3-b]pyridin-2-amine

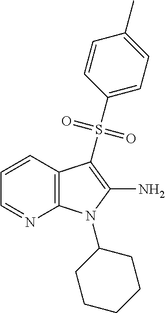 [SMILES: Cc1ccc(S(=O)(=O)c2c(N)n(C3CCCCC3)c3ncccc23)cc1]

A stirred solution of 2-(2-chloropyridin-3-yl)-2-(4-methylbenzenesulfonyl)acetonitrile (Intermediate 11; 600 mg, 2.0 mmol), triethylamine (500 mg, 4.9 mmol) and cyclohexanamine (CAS 108-91-8; 2.43 g, 24.5 mmol) in DMSO (5 mL) was heated to 160° C. for 3 hours in a microwave. The reaction was poured onto ice and extracted with ethyl acetate. The organic phase was concentrated in vacuo. The resulting residue was purified by column chromatography (preparative HPLC, 5-95% acetonitrile/water (with 0.1% ammonia)) to afford the title compound.

¹H NMR (400 MHz, DMSO-d₆) δ ppm 1.20-1.33 (m, 2H) 1.34-1.48 (m, 3H) 1.60-1.71 (m, 3H) 1.78-1.87 (m, 2H) 2.33 (s, 3H) 4.29-4.40 (m, 1H) 6.96-7.09 (m, 3H) 7.32-7.36 (m, 2H) 7.70-7.74 (m, 1H) 7.80-7.85 (m, 2H) 7.92-7.98 (m, 1H)

MS ES⁺: 370

### Example 12 7-(cyclohexanesulfonyl)-5-cyclohexyl-5-pyrrolo[2,3-b]pyrazin-6-amine

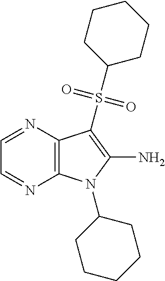 [SMILES: Nc1c(S(=O)(=O)C2CCCCC2)c2nccnc2n1C1CCCCC1]

To a stirred solution of 2,3-dichloropyrazine (CAS 4858-85-9; 1.8 g, 12.1 mmol) and 2-(cyclohexanesulfonyl)acetonitrile (CAS 797036-54-5; 2.7 g, 14.4 mmol) in DMSO (2 mL) was added DBU (1.85 g, 12.1 mmol) and the reaction heated in a microwave to 130° C. for 3 h. To the resulting solution was added triethylamine (600 mg, 59 mmol) and cyclohexanamine (CAS 108-91-8; 6 g, 60.5 mmol) and the reaction heated in a microwave to 170° C. for 3 h. The reaction was poured onto ice and extracted with ethyl acetate. The organic phase was concentrated in vacuo. The resulting residue was purified by column chromatography (preparative HPLC, 5-95% acetonitrile/water (with 0.1% ammonia)) to afford the title compound.

¹H NMR (400 MHz, DMSO-d₆) δ ppm 1.04-1.30 (m, 4H) 1.33-1.49 (m, 4H) 1.55-1.63 (m, 1H) 1.66-1.80 (m, 5H) 1.80-1.98 (m, 4H) 2.39-2.49 (m, 2H) 3.09-3.24 (m, 1H) 4.32-4.44 (m, 1H) 7.31-7.43 (m, 2H) 7.91 (d, J=3 Hz, 1H) 8.09 (d, J=3 Hz, 1H)

MS ES⁺: 363

### Example 13 5-(4,4-difluorocyclohexyl)-7-[(4-methoxybenzene)sulfonyl]-5H-pyrrolo[2,3-b]pyrazin-6-amine

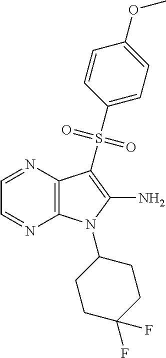 [SMILES: COc1ccc(S(=O)(=O)c2c(N)n(C3CCC(F)(F)CC3)c3nccnc23)cc1]

To a stirred solution of 2-(3-chloropyrazin-2-yl)-2-((4-methoxyphenyl)sulfonyl)acetonitrile (Intermediate 12; 136 mg, 0.420 mmol) and 4,4-difluorocyclohexanamine hydrochloride (CAS 675112-70-6; 433 mg, 2.52 mmol) in N-methyl-2-pyrrolidinone (2 mL) was added triethylamine (0.410 mL, 2.94 mmol). The reaction was then heated in a microwave to 180° C. for 2 h. The reaction mixture was partitioned between water and EtOAc. The phases were separated and the aqueous extracted with EtOAc. The combined organic extracts were then washed with water, dilute citric acid, water, sat. NaHCO₃, sat. brine, dried (H-frit) and evaporated. The crude material was purified by column chromatography (silica, 0-100% EtOAc/petroleum ether) to afford the title compound.

¹H NMR (400 MHz, DICHLOROMETHANE-d₂) δ ppm 1.87-2.11 (m, 4H) 2.24-2.40 (m, 2H) 2.75-2.93 (m, 2H) 3.86 (s, 3H) 4.29-4.44 (m, 1H) 6.20 (br. s., 2H) 6.99 (d, J=9 Hz, 2H) 7.96 (d, J=3 Hz, 1H) 8.11 (d, J=9 Hz, 2H) 8.22 (d, J=3 Hz, 1H)

MS ES⁺: 423

### Example 14 1-(4,4-difluorocyclohexyl)-3-[(4-methoxybenzene)sulfonyl]-1H-pyrrolo[2,3-b]pyridin-2-amine

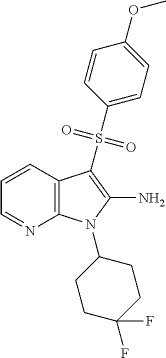 [SMILES: COc1ccc(S(=O)(=O)c2c(N)n(C3CCC(F)(F)CC3)c3ncccc23)cc1]

To a stirred solution of 2-(2-chloropyridin-3-yl)-2-((4-methoxyphenyl)sulfonyl)acetonitrile (Intermediate 13; 210 mg, 0.651 mmol) in N-methyl-2-pyrrolidinone (1 mL) was added a solution of 4,4-difluorocyclohexanamine hydrochloride (CAS 675112-70-6; 670 mg, 3.90 mmol) and triethylamine (0.635 mL, 4.55 mmol) in N-methyl-2-pyrrolidinone (2 mL) and the resulting mixture heated at 165-175° C. for 20 h. The reaction mixture was partitioned between ethyl acetate and water. The phases were separated and the aqueous extracted with ethyl acetate. The combined organics were washed with dilute citric acid, water, sat. aq. sodium bicarbonate solution and brine, dried over MgSO₄, filtered and concentrated in vacuo. The crude product was purified by column chromatography (silica, 0-40% EtOAc/petroleum ether). Further purification was performed by column chromatography (preparative HPLC, 40-80% acetonitrile/water (with 0.1% ammonia)) to afford the title compound.

¹H NMR (4 MHz, CHLOROFORM-d) δ ppm 1.81-2.16 (m, 4H) 2.21-2.49 (m, 2H) 2.53-2.89 (m, 2H) 3.84 (8, 3H) 4.56-4.92 (m, 1H) 5.68 (br. s., 2H) 6.86-7.14 (m, 3H) 7.77-7.99 (m, 3H) 8.00-8.15 (m, 1H)

MS ES⁺: 422

### Example 15 3-(benzenesulfonyl)-1-cyclohexyl-1H-pyrrolo[2,3-b]pyridin-2-amine

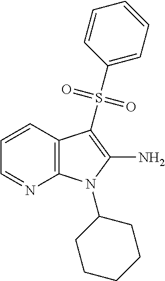 [SMILES: Nc1c(S(=O)(=O)c2ccccc2)c2cccnc2n1C1CCCCC1]

To a stirred solution of 2-(2-chloropyridin-3-yl)-2-(phenylsulfonyl)acetonitrile (Intermediate 14; 100 mg, 0.342 mmol) in N-methyl-2-pyrrolidinone (1 mL) was added a solution of cyclohexanamine (CAS 108-91-8; 0.234 mL, 2.05 mmol) and triethylamine (0.048 mL, 0.342 mmol) in N-methyl-2-pyrrolidinone (1 mL) and the resulting mixture heated at 170° C. for 5 h using a microwave reactor. The reaction mixture was partitioned between ethyl acetate and water. The phases were separated and the aqueous extracted with ethyl acetate. The combined organics were washed with dilute citric acid, water, sat aq. sodium bicarbonate solution and brine, dried over MgSO₄, filtered and concentrated in vacuo. The crude product was purified by column chromatography (silica, 0-40% EtOAc/petroleum ether). Further purification was performed by column chromatography (preparative HPLC, 40-80% acetonitrile/water (with 0.1% ammonia)) to afford the title compound.

¹H NMR (400 MHz, CHLOROFORM-d) δ ppm 0.20-1.56 (m, 3H) 1.72-2.00 (m, 5H) 2.14-2.51 (m, 2H) 4.49 (br. s., 1H) 5.70 (br. s., 2H) 6.89-7.16 (m, 1H) 7.40-7.56 (m, 3H) 7.82-7.91 (m, 1H) 7.92-8.00 (m, 2H) 8.03-8.11 (m, 1H)

MS ES⁺: 356

### Example 16 3-(benzenesulfonyl)-1-cyclohexyl-1H-pyrrolo[2,3-b]pyridin-2-amine

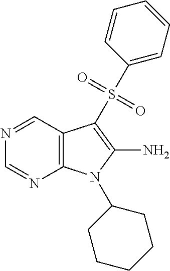 [SMILES: Nc1c(S(=O)(=O)c2ccccc2)c2cncnc2n1C1CCCCC1]

A stirred solution of N-cyclohexyl-5-iodopyrimidin-4-amine (Intermediate 15; 139 mg, 0.459 mmol) and Pd(Ph₃P)₄ (26.5 mg, 0.023 mmol) dry DME (2 mL) was degassed with N₂. In a separate vial 2-(benzenesulfonyl)acetonitrile (CAS 7605-28-9; 91 mg, 0.504 mmol) was dissolved in dry DME (2 mL), degassed and cooled to 0° C. NaH, 60% dispersion in oil (36.7 mg, 0.917 mmol) was added and stirred 5 min. The solution of iodopyrimidine and Pd catalyst was then added via cannula, rinsing with further dry DME. The reaction mixture was then heated in a microwave at 110° C. for 1 h. The reaction mixture was partitioned between EtOAc and water. The aqueous phase was extracted with EtOAc. The combined organic extracts were washed with water, sat. brine, dried (H-frit) and evaporated. The crude material was then purified by column chromatography (silica, 0-40% EtOAc/petroleum ether) to afford the title compound. ¹H NMR (400 MHz, DMSO-d₆) δ ppm 1.32-1.53 (m, 3H) 1.57-1.69 (m, 1H) 1.70-1.90 (m, 4H) 1.98-2.16 (m, 2H) 4.30-4.47 (m, 1H) 7.50-7.65 (m, 5H) 8.01-8.13 (m, 2H) 8.60 (s, 1H) 8.74 (s, 1H)

MS ES⁺: 357

### Example 17 3-(benzenesulfonyl)-1-(4,4-difluorocyclohexyl)-1H-pyrrolo[2,3-b]pyridin-2-amine

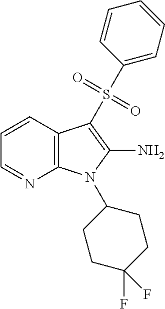 [SMILES: Nc1c(S(=O)(=O)c2ccccc2)c2cccnc2n1C1CCC(F)(F)CC1]

Prepared as described for 3-(benzenesulfonyl)-1-cyclohexyl-1H-pyrrolo[2,3-b]pyridin-2-amine (Example 15), a stirred solution of 2-(2-chloropyridin-3-yl)-2-(phenylsulfonyl)acetonitrile (Intermediate 14; 239 mg, 0.816 mmol), 4,4-difluorocyclohexanamine hydrochloride (CAS 675112-70-6; 662 mg, 4.90 mmol) and triethylamine (0.8 mL, 5.71 mmol) in N-methyl-2-pyrrolidinone (2. mL) was heated at 170° C. for 5 h using a microwave reactor. The crude product was purified by column chromatography (silica, 0-40% EtOAc/petroleum ether). Further purification was performed by column chromatography (preparative HPLC, 40-80% acetonitrile/water (with 0.1% ammonia)) to afford the title compound.

¹H NMR (400 MHz, CHLOROFORM-d) δ ppm 1.82-2.14 (m, 4H) 2.17-2.46 (m, 2H) 2.58-2.87 (m, 2H) 4.49-4.90 (m, 1H) 5.76 (s, 2H) 6.97-7.14 (m, 1H) 7.39-7.63 (m, 3H) 7.76-7.93 (m, 1H) 7.97 (d, J=7 Hz, 2H) 8.04-8.14 (m, 1H)

MS ES⁺: 392

### Example 19 7-(benzenesulfonyl)-5-cyclohexyl-5H-pyrrolo[3,2-d]pyrimidin-6-amine

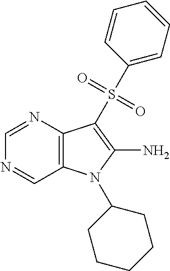 [SMILES: Nc1c(S(=O)(=O)c2ccccc2)c2ncncc2n1C1CCCCC1]

To a stirred degassed solution of 4-chloro-N-cyclohexylpyrimidin-5-amine (Intermediate 16; 209 mg, 0.987 mmol) in dry DME (2 mL) was added Pd(Ph₃P)₄ (29 mg, 0.025 mmol) and Pd(amphos)₂Cl₂ (18 mg, 0.025 mmol). In a separate vial, 2-(benzenesulfonyl)acetonitrile (CAS 7605-28-9; 197 mg, 1.09 mmol) was dissolved in dry DME (2 mL), degassed, cooled in ice and treated with NaH, 60% dispersion in oil (79 mg, 1.98 mmol). The second vial was stirred in ice for 5 min, then at rt for 5 min, under a gentle N₂ stream. The solution of pyrimidine and Pd catalysts was then added via cannula, rinsing with further dry DME. The reaction heated in the microwave at 110° C. for 1 h. The reaction mixture was partitioned between EtOAc and water. The aqueous phase was extracted with EtOAc. The combined organic extracts were washed with water, sat. brine, dried (H-frit) and evaporated. The crude product was purified by column chromatography (silica, 50-90% EtOAc/petroleum ether) to afford the title compound.

¹H NMR (400 MHz, DMSO-d₆) δ ppm 1.32-1.53 (m, 3H) 1.57-1.69 (m, 1H) 1.70-1.90 (m, 4H) 1.98-2.16 (m, 2H) 4.30-4.47 (m, 1H) 7.50-7.65 (m, 5H) 8.01-8.13 (m, 2H) 8.60 (s, 1H) 8.74 (s, 1H)

MS ES⁺: 357

### Example 20 3-(benzenesulfonyl)-1-cyclohexyl-1H-pyrrolo[2,3-]pyridin-2-amine

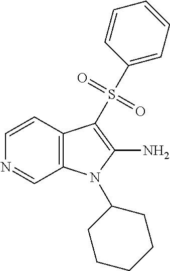 [SMILES: Nc1c(S(=O)(=O)c2ccccc2)c2ccncc2n1C1CCCCC1]

To a stirred solution of 2-(benzenesulfonyl)acetonitrile (CAS 7605-28-9; 330 mg, 1.82 mmol) in DME (3 mL) at 0° C. under a flow of N₂ was added NaH, 60% dispersion in oil (132 mg, 3.31 mmol) and the resulting suspension allowed to stir for 10 min. In a separate flask Pd(Ph₃P)₄ (96 mg, 0.083 mmol) in DME (3 mL) was degassed. The solution in the first flask was added to the solution of Pd(Ph₃P)₄ in DME. After stirring for a further to min N-cyclohexyl-4-iodopyridin-3-amine (Intermediate 17; 500 mg, 1.66 mmol) was added and the reaction mixture subjected to microwave irradiation at 120° C. for 1.5 h. The reaction mixture was poured into water and extracted with ethyl acetate and then the organics washed with brine. The organics were dried over MgSO₄ and concentrated. The crude product was purified by column chromatography (basic silica, 0-20% EtOAc/petroleum ether). The resulting solid was recrystallised from hot IPA/water to afford the title compound.

¹H NMR (400 MHz, DICHLOROMETHANE-d₂) δ ppm 1.28-1.42 (m, 1H), 1.44-1.59 (m, 2H), 1.78-1.89 (m, 1H), 1.92-2.12 (m, 4H), 2.12-2.30 (m, 2H), 3.94-4.10 (m, 1H), 5.89 (br. s., 2H), 7.45-7.62 (m, 4H), 7.89-8.07 (m, 2H), 8.17-8.22 (m, 1H), 8.68 (s, 1H)

MS ES⁺: 356

### Example 21 3-(benzenesulfonyl)-1-(4,4-difluorocyclohexyl)-1H-pyrrolo[3,2-b]pyridin-2-amine

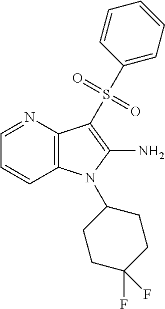 [SMILES: Nc1c(S(=O)(=O)c2ccccc2)c2ncccc2n1C1CCC(F)(F)CC1]

Prepared as described for 3-(benzenesulfonyl)-1-cyclohexyl-1H-pyrrolo[2,3-c]pyridin-2-amine (Example 20), to a stirred solution of 2-(benzenesulfonyl)acetonitrile (CAS 7605-28-9; 548 mg, 3.02 mmol) in DME (3 mL) at 0° C. under a flow of N₂ was added NaH, 60% dispersion in oil (220 mg, 5.50 mmol) and the resulting suspension allowed to stir for to min. In a separate flask Pd(Ph₃P)₄ (159 mg, 00.137 mmol) in DME (3 mL) was degassed. The solution in the first flask was added to the solution of Pd(Ph₃P)₄ in DME. After stirring for a further to min, 2-bromo-N-(4,4-difluorocyclohexyl)pyridin-3-amine (Intermediate 18; 800 mg, 2.75 mmol) was added and the reaction mixture subjected to microwave irradiation at 120° C. for 1.5 h. The crude product was purified by column chromatography (basic silica, 0-50% DCM/EtOAc). The resulting solid was triturated with hot ethanol to afford the title compound. ¹H NMR (400 MHz, DMSO-d₆) δ ppm 1.81-1.93 (m, 2H) 1.95-2.38 (m, 6H) 4.50-4.66 (m, 1H) 6.89-6.97 (m, 1H) 7.17 (s, 2H) 7.47-7.60 (m, 4H) 8.04-8.09 (m, 2H) 8.11-8.15 (m, 1H)

MS ES⁺: 392

### Example 22 1-(4,4-difluorocyclohexyl)-3-[(4-methoxybenzene)sulfonyl]-1H-pyrrolo[3,2-b]pyridin-2-amine

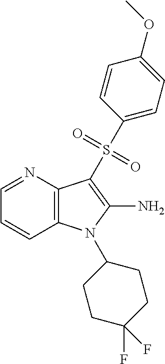 [SMILES: COc1ccc(S(=O)(=O)c2c(N)n(C3CCC(F)(F)CC3)c3cccnc23)cc1]

Prepared as described for 3-(benzenesulfonyl)-1-cyclohexyl-1H-pyrrolo[2,3-c]pyridin-2-amine (Example 20), to a stirred solution of 2-((4-methoxyphenyl)sulfonyl)acetonitrile (CAS 132276-87-o; 638 mg, 3.02 mmol) in DME (4 mL) at 0° C. under a flow of N₂ was added NaH, 60% dispersion in oil (2200 mg, 5.50 mmol) and the resulting suspension allowed to stir for 10 minutes. In a different flask Pd(Ph₃P)₄ (159 mg, 0.137 mmol) in DME (4 mL) was degassed. The solution in the first flask was added to the solution of Pd(Ph₃P)₄ in DME. After stirring for a further to minutes 2-bromo-N-(4,4-difluorocyclohexyl)pyridin-3-amine (Intermediate 18; 800 mg, 2.75 mmol) was added and the reaction mixture subjected to microwave irradiation at 120° C. for 1.5 h. The crude product was purified by column chromatography (basic silica, 0-100% DCM/EtOAc). The resulting solid was triturated with hot ethanol to afford the title compound.

¹H NMR (400 MHz, DICHLOROMETHANE-d₂) δ ppm 1.88-2.12 (m, 4H), 2.27-2.39 (m, 2H), 2.44-2.59 (m, 2H), 3.85 (s, 3H), 4.09-4.28 (m, 1H), 5.93 (s, 2H), 6.89-7.05 (m, 3H), 7.50-7.54 (m, 1H), 8.07-8.20 (m, 2H), 8.30-8.34 (m, 1H)

MS ES⁺: 422

### Example 23 3-(benzenesulfonyl)-1-cyclohexyl-1H-pyrrolo[3,2-c]pyridin-2-amine

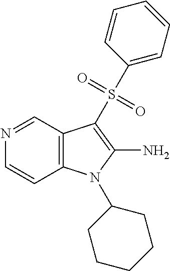 [SMILES: Nc1c(S(=O)(=O)c2ccccc2)c2cnccc2n1C1CCCCC1]

Prepared as described for 3-(benzenesulfonyl)-1-cyclohexyl-1H-pyrrolo[2,3-c]pyridin-2-amine (Example 20), to a stirred degassed solution Pd(Ph₃P)₄ (73 mg, 0.063 mmol) in anhydrous DME (3 mL) under an atmosphere of nitrogen was added a solution of 2-(benzenesulfonyl)acetonitrile (CAS 7605-28-9; 252 mg, 1.39 mmol) and NaH, 60% dispersion in oil (106 mg, 2.65 mmol) in anhydrous DME (4 mL). The resulting mixture was stirred at room temperature for 10 min followed by addition of a solution of 3-bromo-N-cyclohexylpyridin-4-amine (Intermediate 19; 322 mg, 1.262 mmol) in anhydrous DME (1 mL). The reaction mixture was heated at 120° C. for 1.5 h. Purification was carried out by column chromatography (silica, 0-100% EtOAc/petroleum ether). Further purification was performed by column chromatography (preparative HPLC, 20-600% acetonitrile/water (with 0.1% formic acid)) to afford the title compound.

¹H NMR (400 MHz, CHLOROFORM-d) δ ppm 1.19-1.55 (m, 3H) 1.76-2.29 (m, 7H) 3.82-4.19 (m, 1H) 5.75 (br. s, 2H) 7.20 (d, J=6 Hz, 1H) 7.38-7.62 (m, 3H) 7.88-8.09 (m, 2H) 8.23 (d, J=6 Hz, 1H) 8.92 (s, 1H)

MS ES⁺: 356

### Example 24 methyl N-[7-(benzenesulfonyl)-5-cyclohexyl-5H-pyrrolo[2,3-b]pyrazin-6-yl]carbamate

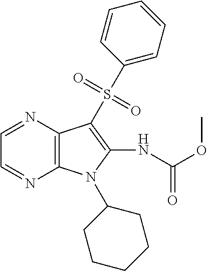 [SMILES: COC(=O)Nc1c(S(=O)(=O)c2ccccc2)c2nccnc2n1C1CCCCC1]

To a stirred solution of methyl N-[7-(benzenesulfonyl)-5-cyclohexyl-5H-pyrrolo[2,3-b]pyrazin-6-yl]-N-(methoxycarbonyl)carbamate (Intermediate 20; 0.214 g, 0.453 mmol) in MeOH (7 mL) was added sodium methanolate (16 mg, 0.3 mmol) and the resulting mixture stirred at room temperature for 3 h. A further portion of sodium methoxide (10 mg, 0.19 mmol) was added and the reaction was stirred at room temperature for a further 2 h. The solvent was removed under reduced pressure. The residue was partitioned between DCM and water, passed through a phase separator and concentrated in vacuo. Purification was performed by column chromatography (preparative HPLC, 10-50% acetonitrile/water (with 0.1% ammonia)) to afford the title compound.

¹H NMR (4000 MHz, CHLOROFORM-d) δ ppm 1.27-1.46 (m, 3H) 1.66-2.05 (m, 5H) 2.47-2.73 (m, 2H) 3.89 (s, 3H) 4.09-4.28 (m, 1H) 7.37-7.64 (m, 3H) 8.05-8.21 (m, 2H) 8.27 (d, J=3 Hz, 2H) 8.52 (d, J=3 Hz, 1H)

MS ES⁺: 415

### Example 25 3-(benzenesulfonyl)-1-(4,4-difluorocyclohexyl)-6-methyl-1H-pyrrolo[2,3-b]pyridin-2-amine

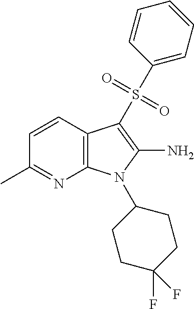 [SMILES: Cc1ccc2c(S(=O)(=O)c3ccccc3)c(N)n(C3CCC(F)(F)CC3)c2n1]

To a stirred solution 2-amino-1-(4,4-difluorocyclohexyl)-6-methyl-3-(phenylsulfonyl)-1H-pyrrolo[2,3-b]pyridine 7-oxide (Intermediate 22; 65 mg, 0.154 mmol) in chloroform (2 mL) under an atmosphere of nitrogen was added trichlorophosphine (0.1 mL, 1.15 mmol). The resulting mixture was heated at reflux for 1 h. The mixture was partitioned between DCM and saturated NaHCO₃. The phases were separated and the aqueous extracted with DCM. The combined organics were washed with saturated NaHCO₃, dried over MgSO₄ and concentrated in vacuo. The crude product was purified by column chromatography (preparative HPLC, 40-80% acetonitrile/water (with 0.1% ammonia)) to afford the title compound.

¹H NMR (400 MHz, CHLOROFORM-d) δ ppm 1.76-2.19 (m, 4H) 2.22-2.41 (m, 2H) 2.52 (s, 3H) 2.58-2.81 (m, 2H) 4.58-4.87 (m, 1H) 5.57 (br. s, 2H) 6.91 (d, J=8 Hz, 1H) 7.36-7.60 (m, 3H) 7.76 (d, J=8 Hz, 1H) 7.86-8.07 (m, 2H)

MS ES⁺: 406

### Example 26 7-(benzenesulfonyl)-5-cyclohexyl-4-methoxy-5H-pyrrolo[3,2-d]pyrimidin-6-amine

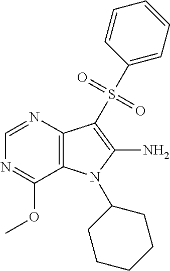 [SMILES: COc1ncnc2c(S(=O)(=O)c3ccccc3)c(N)n(C3CCCCC3)c12]

Prepared as described for 7-(benzenesulfonyl)-5-cyclohexyl-5H-pyrrolo[3,2-d]pyrimidin-6-amine (Example 19), to a stirred degassed solution of Pd(Ph₃P)₄ (14 mg, 0.013 mmol) and Pd(amphos)₂Cl₂ (9 mg, 0.013 mmol) in anhydrous DME (1 mL) under an atmosphere of nitrogen was added a solution of 2-(benzenesulfonyl)acetonitrile (CAS 7605-28-9; 100 mg, 0.550 mmol) and NaH, 60% dispersion in oil (44.0 mg, 1.100 mmol) in anhydrous DME (1 mL). The resulting mixture was stirred at room temperature for 10 min followed by addition of a solution of 4-chloro-N-cyclohexyl-6-methoxypyrimidin-5-amine (Intermediate 23; 121 mg, 0.5 mmol) in anhydrous DME (1 mL). The reaction mixture was heated at 120° C. for 1.5 h. The crude product was purified by column chromatography (preparative HPLC, 30-70% acetonitrile/water (with 0.1% ammonia)) to afford the title compound.

¹H NMR (400 MHz, CHLOROFORM-d) δ ppm 1.13-1.53 (m, 4H) 1.65-2.51 (m, 7H) 4.09 (s, 3H) 5.86 (br. s., 2H) 7.42-7.60 (m, 3H) 8.14-8.30 (m, 2H) 8.51 (s, 1H)

MS ES⁺: 387

### Example 27 5-(benzenesulfonyl)-3-chloro-7-cyclohexyl-7H-pyrrolo[2,3-c]pyridazin-6-amine

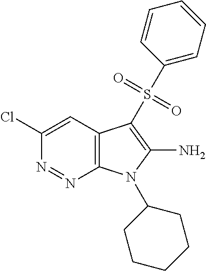 [SMILES: Nc1c(S(=O)(=O)c2ccccc2)c2cc(Cl)nnc2n1C1CCCCC1]

Prepared as described for 3-(benzenesulfonyl)-1-cyclohexyl-1H-pyrrolo[2,3-c]pyridin-2-amine (Example 20), to a solution of 2-(benzenesulfonyl)acetonitrile (CAS 7605-28-9; 34 mg, 0.189 mmol) in DME (3 mL) at 0° C. was added NaH, 60% dispersion in oil (14 mg, 0.344 mmol). After 10 minutes the resulting suspension was added to a degassed solution of Pd(Ph₃P)₄ (10 mg, 8.60 μmol) in DME (2 mL). The resulting suspension was allowed to stir at room temperature for 20 minutes. 4-bromo-6-chloro-N-cyclohexylpyridazin-3-amine (Intermediate 24; 50 mg, 0.172 mmol) was then added and the reaction mixture subjected to microwave irradiation at 120° C. for 2 h. Purification was carried out by column chromatography (silica, 0-50% EtOAc/petroleum ether) to afford the title compound.

¹H NMR (400 MHz, DMSO-d₆) δ ppm 1.18-1.49 (m, 3H), 1.62-1.7 (m, 3H), 1.77-1.86 (m, 2H), 2.42-2.49 (m, 2H), 4.43 (br. s., 1H), 7.47 (s, 1H), 7.55-7.70 (m, 3H), 7.96 (br. s., 2H), 8.00-8.08 (m, 2H)

MS ES⁺: 391

### Example 28 5-(benzenesulfonyl)-7-cyclohexyl-7H-pyrrolo[2,3-c]pyridazin-6-amine

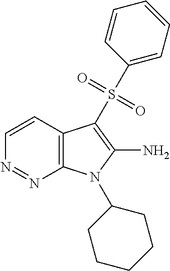 [SMILES: Nc1c(S(=O)(=O)c2ccccc2)c2ccnnc2n1C1CCCCC1]

A solution of 5-(benzenesulfonyl)-3-chloro-7-cyclohexyl-7H-pyrrolo[2,3-c]pyridazin-6-amine (Example 27; 31 mg, 0.079 mmol) in THF (2 mL) was passed through an H-Cube using a 10% Palladium on carbon cat-cart at 40° C. at ‘full H₂’. The reactant was cycled through the H-Cube for 2 h at 1 mL/min. The product solution was then concentrated in vacuo. Purification was carried out by column chromatography (silica, 0-50% EtOAc/petroleum ether) followed by column chromatography (silica, 0-10% MeOH/DCM) and finally by trituration with diethyl ether to afford the title compound.

¹H NMR (400 MHz, DICHLOROMETHANE-d₂) δ ppm 1.25-1.44 (m, 3H), 1.62-1.71 (m, 1H), 1.75-1.98 (m, 4H), 2.34-2.48 (m, 2H), 4.32 (br. s., 1H), 6.13 (br. s, 2H), 7.31-7.55 (m, 4H), 7.78-7.89 (m, 2H), 8.57-8.67 (m, 1H)

MS ES⁺: 357

### Example 29 7-(benzenesulfonyl)-5-(4,4-difluorocyclohexyl)-4-ethoxy-5H-pyrrolo[3,2-d]pyrimidin-6-amine

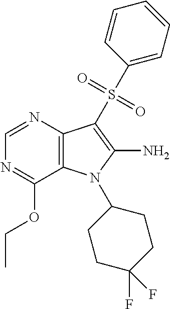 [SMILES: CCOc1ncnc2c(S(=O)(=O)c3ccccc3)c(N)n(C3CCC(F)(F)CC3)c12]

As described for 7-(benzenesulfonyl)-5-cyclohexyl-5H-pyrrolo[3,2-d]pyrimidin-6-amine (Example 19), to a solution of 2-(benzenesulfonyl)acetonitrile (CAS 7605-28-9; 373 mg, 2.06 mmol) in DME (3 mL) at 0° C. was added NaH, 60% dispersion in oil (165 mg, 4.11 mmol). After 10 min the resulting suspension was added to a degassed solution of Pd(Ph₃P)₄ (59 mg, 0.051 mmol) and Pd(amphos)₂C₂ (36 mg, 0.051 mmol) in DME (2 mL). The resulting suspension was allowed to stir at room temperature for 20 minutes. 4-chloro-N-(4,4-difluorocyclohexyl)-6-ethoxypyrimidin-5-amine (Intermediate 25; 6 mg, 2.06 mmol) was then added and the reaction mixture subjected to microwave irradiation at 120° C. for 2 b. Purification was carried out by column chromatography (silica, 0-50% EtOAc/petroleum ether) to afford the title compound.

¹H NMR (400 MHz, DMSO-d₆) δ ppm 1.34-1.41 (m, 3H), 1.71-1.80 (m, 2H), 1.92-2.21 (m, 4H), 2.44-2.61 (m, 2H), 4.43-4.45 (m, 3H), 7.34 (br. s, 2H), 7.52-7.64 (m, 3H), 8.00-8.10 (nm, 2H), 8.30 (s, 1H)

MS ES⁺: 437

### Example 30 7-(benzenesulfonyl)-4-(benzyloxy)-5-(4,4-difluorocyclohexyl)-5H-pyrrolo[3,2-d]pyrimidin-6-amine

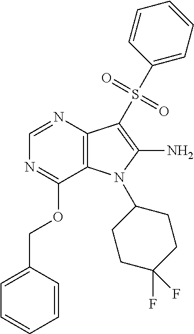 [SMILES: Nc1c(S(=O)(=O)c2ccccc2)c2ncnc(OCc3ccccc3)c2n1C1CCC(F)(F)CC1]

As described for 7-(benzenesulfonyl)-5-cyclohexyl-5H-pyrrolo[3,2-d]pyrimidin-6-amine (Example 19), to a solution of 2-(benzenesulfonyl)acetonitrile (CAS 7605-28-9; 512 mg, 2.83 mmol) in DME (3 mL) at 0° C. was added NaH, 60% dispersion in oil (226 mg, 5.65 mmol). After to min the resulting suspension was added to a degassed solution of Pd(Ph₃P)₄ (0.082 g, 0.071 mmol) and Pd(amphos)₂Cl₂ (0.050 g 0.071 mmol) in DME (2 mL). The resulting suspension was allowed to stir at room temperature for 20 minutes. 4-(benzyloxy)-6-chloro-N-(4,4-difluorocyclohexyl)pyrimidin-5-amine (Intermediate 27; 1 g, 2.83 mmol) was then added and the reaction mixture subjected to microwave irradiation at 120° C. for 2 h. Purification was carried out by column chromatography (silica, 0-30% EtOAc/petroleum ether) to afford the title compound.

¹H NMR (400 MHz, DMSO-d₆) δ ppm 1.64-1.78 (m, 2H), 0.83-2.11 (m, 4H), 2.40-2.58 (m, 2H), 4.44-4.57 (m, 1H), 5.55 (s, 2H), 7.26-7.39 (m, 5H), 7.42-7.48 (m, 2H), 7.51-7.66 (m, 3H), 8.02-8.10 (m, 2H), 8.32 (s, 1H)

MS ES⁺: 499

### Example 31 6-amino-5-(4,4-difluorocyclohexyl)-7-(phenylsulfonyl)-5H-pyrrolo[3,2-d]pyrimidin-4-ol

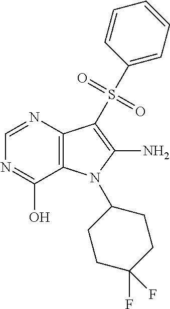 [SMILES: Nc1c(S(=O)(=O)c2ccccc2)c2ncnc(O)c2n1C1CCC(F)(F)CC1]

A solution of 7-(benzenesulfonyl)-4-(benzyloxy)-5-(4,4-difluorocyclohexyl)-5H-pyrrolo[3,2-d]pyrimidin-6-amine (Example 30; 420 mg, 0.842 mmol) in methanol (17 mL) was passed through an H-Cube using a palladium on carbon (10%) cat-cart at ‘full H₂’ at room temperature at 1 mL/min. The product solution was concentrated and triturated with ethyl acetate to afford the title compound.

¹H NMR (4 MHz, DMSO-d₆) δ ppm 1.61-1.71 (m, 2H), 1.85-2.18 (m, 4H), 2.68-2.83 (m, 2H), 4.32-4.52 (m, 1H), 6.94 (s, 2H), 7.51-7.68 (m, 3H), 7.86 (s, 1H), 7.94-8.12 (m, 2H), 12.04 (br. s., 1H)

MS ES⁺: 409

### Example 32 7-(benzenesulfonyl)-4-chloro-5-(4,4-difluorocyclohexyl)-5H-pyrrolo[3,2-d]pyrimidin-6-amine

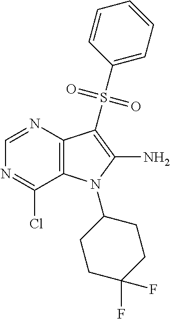 [SMILES: Nc1c(S(=O)(=O)c2ccccc2)c2ncnc(Cl)c2n1C1CCC(F)(F)CC1]

To a stirred suspension of 6-amino-7-(benzenesulfonyl)-5-(4,4-difluorocyclohexyl)-3H-pyrrolo[3,2-d]pyrimidin-4(5H)-one (Example 31; 75 mg, 0.184 mmol) in POCl₃ (1 mL, 10.7 mmol) was heated at 80° C. overnight. The reaction was quenched slowly into warm water. The resulting solution was basified to pH₁₂ with 2M NaOH. The resulting aqueous mixture was extracted with DCM. The organics were separated and concentrated. Trituration with diethyl ether afforded the title compound.

¹H NMR (400 MHz, DICHLOROMETHANE-d₂) δ ppm 1.86-2.23 (m, 4H), 2.27-2.68 (m, 4H), 5.41-5.73 (m, 1H), 6.28 (br. s., 2H), 7.35-7.73 (m, 3H), 8.05-8.32 (m, 2H), 8.56 (br. s., 1H)

MS ES⁺: 427

### Example 33 7-(benzenesulfonyl)-5-(4,4-difluorocyclohexyl)-4-N-methyl-5H-pyrrolo[3,2-d]pyrimidine-4,6-diamine

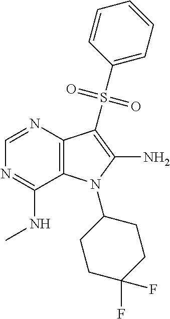 [SMILES: CNc1ncnc2c(S(=O)(=O)c3ccccc3)c(N)n(C3CCC(F)(F)CC3)c12]

A solution of 7-(benzenesulfonyl)-4-chloro-5-(4,4-difluorocyclohexyl)-5H-pyrrolo[3,2-d]pyrimidin-6-amine (Example 32; 40 mg, 0.094 mmol) and methanamine, 2M in THF (0.234 mL, 0.469 mmol) in THF (1 mL) was subjected to microwave irradiation at 120-160° C. for a total of 7 h. The reaction mixture was concentrated in vacuo. To the crude product was added methanamine 2M in THF (2 ml). The solution was subjected to microwave irradiation for a further 2 h at 160° C. The reaction mixture was poured into sat. NaHCO₃ and extracted with DCM. The organics were separated and concentrated to afford the title compound.

¹H NMR (4 MHz, DMSO-d₆) δ ppm 1.95-2.04 (m, 2H), 2.06-2.30 (m, 4H), 2.33-2.48 (m, 2H), 2.95 (d, J=5 Hz, 3H), 4.45-4.58 (m, 1H), 5.84-5.91 (m, 1H), 6.82 (s, 2H), 7.51-7.73 (m, 3H), 8.04-8.15 (m, 2H), 8.23 (s, 1H)

MS ES⁺: 422

### Example 34 7-(benzenesulfonyl)-5-cyclohexyl-4-N,4-N-dimethyl-5H-pyrrolo[3,2-d]pyrimidine-4,6-diamine

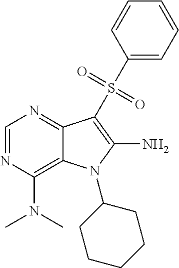 [SMILES: CN(C)c1ncnc2c(S(=O)(=O)c3ccccc3)c(N)n(C3CCCCC3)c12]

As described for 7-(benzenesulfonyl)-5-cyclohexyl-5H-pyrrolo[3,2-d]pyrimidin-6-amine (Example 19), to a stirred degassed solution of Pd(Ph₃P)₄ (28 mg, 0.024 mmol) and Pd(amphos)₂Cl₂ (17 mg, 0.024 mmol) in anhydrous DME (3 mL) under an atmosphere of nitrogen was added a solution of 2-(benzenesulfonyl)acetonitrile (CAS 7605-28-9; 261 mg, 1.44 mmol) and NaH, 60% dispersion in oil (115 mg, 2.89 mmol) in anhydrous DME (3 mL). The resulting mixture was stirred at room temperature for to min followed by addition of a solution of 6-chloro-5-N-cyclohexyl-4-N,4-N-dimethylpyrimidine-4,5-diamine (Intermediate 28; 245 mg, 0.962 mmol) in anhydrous DME (3 mL). The reaction mixture was heated at 125° C. for 20 h. The crude product was purified by column chromatography (preparative HPLC, 40-80% acetonitrile/water (with 0.1% ammonia)) to afford the title compound.

¹H NMR (400 MHz, CHLOROFORM-d) δ ppm 1.03-2.10 (m, 10H) 2.89 (s, 6H) 4.67-4.92 (m, 1H) 6.01 (br. s., 2H) 7.39-7.63 (m, 3H) 8.11-8.32 (m, 2H) 8.53 (s, 1H)

MS ES⁺: 400

### Example 35 7-(benzenesulfonyl)-5-cyclopentyl-4-methoxy-5H-pyrrolo[3,2-d]pyrimidin-6-amine

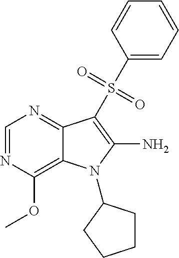 [SMILES: COc1ncnc2c(S(=O)(=O)c3ccccc3)c(N)n(C3CCCC3)c12]

As described for 7-(benzenesulfonyl)-5-cyclohexyl-5H-pyrrolo[3,2-d]pyrimidin-6-amine (Example 19), to a stirred degassed solution of Pd(Ph₃P)₄ (32 mg, 0.027 mmol) and Pd(amphos)₂Cl₂ (19 mg, 0.027 mmol) in anhydrous DME (2 mL) under an atmosphere of nitrogen was added a solution of 2-(benzenesulfonyl)acetonitrile (CAS 7605-28-9; 298 mg, 1.65 mmol) and NaH, 60% dispersion in oil (132 mg, 3.29 mmol) in anhydrous DME (2 mL). The resulting mixture was stirred at room temperature for to min followed by addition of a solution of 4-chloro-N-cyclopentyl-6-methoxypyrimidin-5-amine (Intermediate 29; 250 mg, 1.10 mmol) in anhydrous DME (2 mL). The reaction mixture was heated at 120° C. for 16 h. The crude product was purified by recrystallisation from DMSO/MeOH (1:1) to afford the title compound. ¹H NMR (400 MHz, DMSO-d₆) δ ppm 1.46-1.73 (m, 2H) 1.80-2.05 (m, 6H) 3.98 (s, 3H) 4.70-5.01 (m, 1H) 7.25 (br. s, 2H) 7.42-7.72 (m, 3H) 7.94-8.13 (m, 2H) 8.33 (s, 1H)

MS ES⁺: 373

### Example 36 3-(benzenesulfonyl)-1-cyclohexyl-7-methoxy-1-pyrrolo[2,3-c]pyridin-2-amine

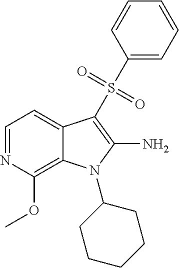 [SMILES: COc1nccc2c(S(=O)(=O)c3ccccc3)c(N)n(C3CCCCC3)c12]

As described for 7-(benzenesulfonyl)-5-cyclohexyl-5H-pyrrolo[3,2-d]pyrimidin-6-amine (Example 19), to a stirred degassed solution of Pd(Ph₃P)₄ (23.64 mg, 0.0020 mmol) and Pd(amphos)₂Cl₂ (19 mg, 0.027 mmol) in anhydrous DME (2 mL) under an atmosphere of nitrogen was added a solution of 2-(benzenesulfonyl)acetonitrile (CAS 7605-28-9; 148 mg, 0.818 mmol) and NaH, 60% dispersion in oil (65.5 mg, 1.637 mmol) in anhydrous DME (3 mL). The resulting mixture was stirred at room temperature for to min followed by addition of a solution of 4-chloro-N-cyclohexyl-2-methoxypyridin-3-amine (Intermediate 30; 197 mg, 0.818 mmol) in anhydrous DME (1 mL). The reaction mixture was subjected to microwave irradiation at 120° C. for 2 h. Purification was carried out by column chromatography (C18-silica 5-95% methanol/water+0.1% ammonia) to afford the title compound.

¹H NMR (4 MHz, DICHLOROMETHANE-d₂) δ ppm 1.06-1.44 (m, 4H), 1.59-2.10 (m, 6H), 2.14-2.58 (m, 1H), 3.91 (s, 3H), 5.58 (br. s., 2H), 7.10 (d, J=5 Hz, 1H), 7.32-7.46 (m, 3H), 7.63 (d, J=5 Hz, 1H), 7.79-7.87 (m, 2H)

MS ES⁺: 386

### Example 37 6-amino-7-(benzenesulfonyl)-5-cyclohexyl-5H-pyrrolo[3,2-d]pyrimidine-4-carbonitrile

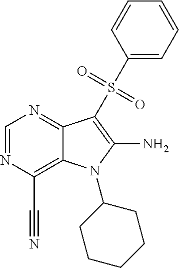 [SMILES: N#Cc1ncnc2c(S(=O)(=O)c3ccccc3)c(N)n(C3CCCCC3)c12]

A stirred suspension of dicyanozinc (CAS 557-21-1; 18 mg, 0.153 mmol), Pd(Ph₃P)₄ (30 mg, 0.026 mmol) and 7-(benzenesulfonyl)-4-chloro-5-cyclohexyl-5H-pyrrolo[3,2-d]pyrimidin-6-amine (Intermediate 34; 100 mg, 0.256 mmol) in N,N-dimethylformamide (1 mL) was subjected to microwave irradiation at 150° C. for 30 minutes. The reaction mixture was poured into sat. NaHCO₃ solution and extracted with ethyl acetate. The organics were washed with brine, dried over MgSO₄ and concentrated to afford the title compound.

¹H NMR (400 MHz, DMSO-d₆) δ ppm 1.37-1.50 (m, 3H) 1.55-1.72 (m, 1H) 1.80-2.00 (m, 4H) 2.24-2.41 (m, 2H) 4.55-4.82 (m, 1H) 7.52-7.69 (m, 3H) 7.97 (br.s., 2H) 8.05-8.11 (m, 2H) 8.67 (s, 1H).

MS ES⁺: 382

### Example 38 5-cyclohexyl-7-(2-fluorobenzenesulfonyl)-4-methoxy-2-methyl-5H-pyrrolo[3,2-d]pyrimidin-6-amine

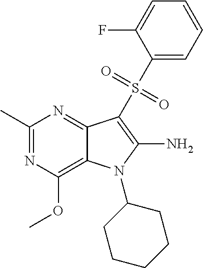 [SMILES: COc1nc(C)nc2c(S(=O)(=O)c3ccccc3F)c(N)n(C3CCCCC3)c12]

As described for 7-(benzenesulfonyl)-5-cyclohexyl-5H-pyrrolo[3,2-d]pyrimidin-6-amine (Example 19), to a solution of 2-(2-fluorobenzenesufonyl)acetonitrile (CAS 59849-52-4; 195 mg, 0.978 mmol) in DME (1 mL) was added NaH, 60% dispersion in oil (86 mg, 2.15 mmol). In a separate flask Pd(Ph₃P)₄ (28 mg, 0.024 mmol), Pd(amphos)₂Cl₂ (17 mg, 0.024 mmol) and 4-chloro-N-cyclohexyl-6-methoxy-2-methylpyrimidin-5-amine (Intermediate 35; 250 mg, 0.978 mmol) were stirred in DME (2 mL) and degassed. To the catalyst/substrate mixture was added the preformed sodium salt of 2-(2-fluorobenzenesulfonyl)acetonitrile and the reaction subjected to microwave irradiation at 130° C. for 2 h. Purification was carried out by column chromatography (silica, 0-10% MeOH/DCM) followed by trituration with ethyl acetate to afford the title compound.

¹H NMR (400 MHz, DMSO-d₆) δ ppm 1.21-1.31 (m, 1H) 1.36-1.49 (m, 2H) 1.63-1.70 (m, 3H) 1.79-1.88 (m, 2H) 2.12-2.24 (m, 2H) 2.35 (s, 3H) 3.97 (s, 3H) 4.28-4.48 (m, 1H) 7.17 (br. s., 2H) 7.27-7.34 (m, 1H) 7.36-7.42 (m, 1H) 7.60-7.70 (m, 1H) 8.01-8.07 (m, 1H)

MS ES⁺: 419

### Example 39 5-cyclohexyl-7-(3-fluorobenzenesulfonyl)-4-methoxy-2-methyl-5H-pyrrolo[3,2-d]pyrimidin-6-amine

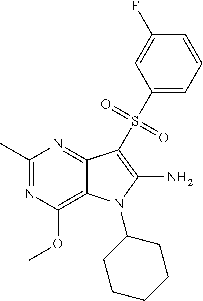 [SMILES: COc1nc(C)nc2c(S(=O)(=O)c3cccc(F)c3)c(N)n(C3CCCCC3)c12]

As described for 7-(benzenesulfonyl)-5-cyclohexyl-5H-pyrrolo[3,2-d]pyrimidin-6-amine (Example 19), to a solution of 2-(3-fluorobenzenesulfonyl)acetonitrile (CAS 61081-29-6; 300 mg, 1.51 mmol) in dioxane (3 mL) was added NaH, 60% dispersion in oil (133 mg, 3.31 mmol). In a separate flask Pd(Ph₃P)₄ (70 mg, 0.060 mmol), Pd(amphos)₂Cl₂ (43 mg, 0.060 mmol) and 4-chloro-N-cyclohexyl-6-methoxy-2-methylpyrimidin-5-amine (Intermediate 35; 385 mg, 0.978 mmol) were stirred in dioxane (2 mL) and degassed. To the catalyst/substrate mixture was added the preformed sodium salt of 2-(3-fluorobenzenesulfonyl)acetonitrile and the reaction heated at reflux for 3 h. Purification was carried out by column chromatography (C18-silica 5-95% methanol/water+0.1% ammonia).

¹H NMR (400 MHz, DMSO-d₆) δ ppm 1.20-1.30 (m, 1H) 1.33-1.47 (m, 2H) 1.58-1.70 (m, 3H) 1.77-1.84 (m, 2H) 2.08-2.21 (m, 2H) 2.49 (s, 3H) 3.99 (s, 3H) 4.25-4.46 (m, 1H) 7.19 (br. s., 2H) 7.42-7.51 (m, 1H) 7.57-7.65 (m, 1H) 7.83-8.00 (m, 2H)

MS ES⁺: 419

### Example 40 7-(benzenesulfonyl)-4-methoxy-5-(oxan-4-yl)-5H-pyrrolo[3,2-d]pyrimidin-6-amine

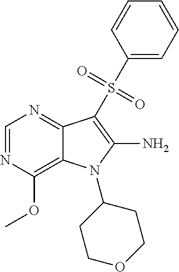 [SMILES: COc1ncnc2c(S(=O)(=O)c3ccccc3)c(N)n(C3CCOCC3)c12]

A stirred solution of 4-chloro-6-methoxy-N-(oxan-4-yl)pyrimidin-5-amine (249 mg, 1.02 mmol), 2-(benzenesulfonyl)acetonitrile (CAS 7605-28-9; 204 mg, 1.12 mmol), Pd(Ph₃P)₄ (59 mg, 0.051 mmol) and Pd(amphos)₂Cl₂ (36 mg, 0.051 mmol) in Dioxane (5 mL) was degassed for 5 minutes. NaHMDS solution (2M in THF, 1.53 mL, 3.07 mmol) was added and the mixture was heated to reflux for 1.5 h. The mixture was partitioned between ethyl acetate and sat. aq. NaHCO₃ then the organic phase was washed with brine and dried over MgSO₄ and then concentrated in vacuo. Purification was carried out by column chromatography (silica, 0-100% EtOAc/Petrol then 0-10% MeOH/DCM). Further purification was carried out by column chromatography (preparative HPLC, 20-60% acetonitrile/water (with 0.1% ammonia)) to afford the title compound.

¹H NMR (400 MHz, DMSO-d₆) δ ppm 1.57-1.66 (m, 2H) 2.34-2.47 (m, 2H) 3.45 (t, J=11 Hz, 2H) 3.94-4.03 (m, 5H) 4.56-4.67 (m, 1H) 7.32 (br. s, 2H) 7.51-7.62 (m, 3H) 8.02-8.08 (m, 2H) 8.32 (s, 1H)

MS ES⁺: 389

### Example 41 6-amino-5-cyclohexyl-N-phenyl-5H-pyrrolo[2,3-b]pyrazine-7-sulfonamide

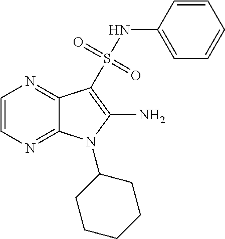 [SMILES: Nc1c(S(=O)(=O)Nc2ccccc2)c2nccnc2n1C1CCCCC1]

To a solution of 5-cyclohexyl-6-(1,3-dioxo-2,3-dihydro-1H-isoindol-2-yl)-N-phenyl-5H-pyrrolo[2,3-b]pyrazine-7-sulfonamide (Intermediate 43; 46 mg, 0.092 mmol) in EtOH (1 mL) was added hydrazine monohydrate (13 μL, 0.275 mmol) and the reaction mixture stirred at reflux overnight. The reaction mixture was filtered and the resulting solid washed with methanol. The combined filtrates were concentrated in vacuo. Purification was carried out by column chromatography (silica, 0-50% EtOAc/petroleum ether) to afford the title compound.

¹H NMR (400 MHz, DICHLOROMETHANE-d₂) δ ppm 1.21-1.49 (m, 4H) 1.67-1.82 (m, 2H) 1.84-1.97 (m, 2H) 2.23-2.39 (m, 2H) 4.04-4.18 (m, 1H) 5.77 (br. s., 2H) 6.97-7.07 (m, 3H) 7.10-7.20 (m, 2H) 7.32 (br. s., 1H) 7.90-7.97 (m, 1H) 8.14-8.22 (m, 1H)

MS ES⁺: 372

### Example 42 6-amino-5-cyclohexyl-N-(pyridin-3-yl)-5H-pyrrolo[2,3-b]pyrazine-7-sulfonamide

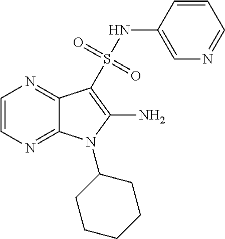 [SMILES: Nc1c(S(=O)(=O)Nc2cccnc2)c2nccnc2n1C1CCCCC1]

To a solution of 5-cyclohexyl-6-(1,3-dioxo-2,3-dihydro-1H-isoindol-2-yl)-N-(pyridin-3-yl)-5H-pyrrolo[2,3-b]pyrazine-7-sulfonamide (Intermediate 43; 20 mg, 0.040 mmol) in EtOH (1 mL) was added hydrazine monohydrate (6 μL, 0.119 mmol) and the reaction mixture stirred at reflux overnight. The reaction mixture was filtered and the resulting solid washed with methanol. The combined filtrates were concentrated in vacuo. Purification was carried out by column chromatography (silica, 0-50% EtOAc/petroleum ether) to afford the title compound. ¹H NMR (4 MHz, DMSO-d₆) δ ppm 1.20-129 (m, 1H) 1.33-1.50 (m, 2H) 0.61-1.73 (m, 3H) 1.75-1.88 (m, 2H) 2.35-2.48 (m, 2H) 4.30-4.43 (m, 1H) 7.12-7.22 (m, 1H) 7.36-7.47 (m, 3H) 7.84-7.93 (m, 1H) 8.02-8.13 (m, 2H) 8.21-8.8 (m, 1H) 10.42 (s, 1H)

MS ES⁺: 373

Examples 43 to 56 (see Table 2 following) were prepared according to one of the procedures 1, 2 or 3 described below.

### Procedure 1

A solution of 2-(benzenesulfonyl)-2-(3-chloropyrazin-2-yl)acetonitrile (Intermediate 1; 100 mg, 0.34 mmol) in NMP (0.5 mL) was treated with a primary amine (0.68 mmol) and heated in the microwave at 170° C. for 1 h. Where the amine was used as a hydrochloride salt, triethylamine (0.095 mL, 0.68 mmol, 2 eq.) was included in the reaction. A further portion of each amine (1.14 mmol, 3 eq) was added and heating was repeated as before. The reaction mixtures were purified directly by preparative HPLC using one of the methods listed below.

### Procedure 2

A solution of 2-(benzenesulfonyl)-2-(3-chloropyrazin-2-yl)acetonitrile (Intermediate 1; 110 mg, 0.326 mmol) in DMSO (1 mL) was treated with a primary amine (1.96 mmol, 6 eq.) and triethylamine (0.045 mL, 0.326 mmol) and heated to 180° C. for 3 h. The reaction mixtures were diluted with DMSO (2 mL), filtered and purified by preparative HPLC using one of the methods listed below.

### Procedure 3

A solution of 2-(benzenesulfonyl)-2-(3-chloropyrazin-2-yl)acetonitrile (Intermediate 1; 70 mg, 0.238 mmol) in NMP (1.0 mL) was treated with a primary amine (1.43 mmol) and triethylamine (0.033 mL, 0.238 mmol) and heated in the microwave at 180° C. for 2.5 h. Where the amine used was a hydrochloride salt, triethylamine (0.196 mmol, 1.43 mmol) was included in the reaction. Samples were typically diluted with DMSO, filtered and purified by preparative HPLC using one of the methods listed below. If an aqueous workup was necessary, the reaction mixture was diluted with water and extracted with EtOAc). The combined extracts were washed with citric acid solution, water, sodium bicarbonate solution, water and brine then dried (H-frit) and evaporated, with the crude product then being purified by preparative HPLC using one of the methods listed below.

| HPLC | Gradient (acetonitrile/water |
|---|---|
| Method | (with 0.1% ammonia)) |
| A | 5-25% |
| B | 5-40% |
| C | 10-50% |
| D | 20-60% |
| E | 30-70% |
| F | 40-80% |
| G | 55-95% |

TABLE 2

| | | | | Syn- | Purifi- | | |
|---|---|---|---|---|---|---|---|
| | | | | thesis | cation | | |
| Ex. | | | | meth- | meth- | MS | ¹H NMR data |
| No. | Compound name | Structure | Starting amine | od | od | ES+ | δ ppm |
| 43 | 5-cyclobutyl-7- (phenylsulfonyl)-5H- pyrrolo[2,3-b]pyrazin-6- amine | 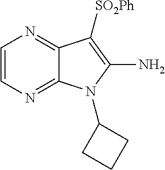 | Cyclobutanamine (CAS 2516-34-9) | 1 | D | 329 | (400 MHz, DMSO-d₆) 1.65-1.82 (m, 1 H) 1.84-1.95 (m, 1 H) 2.24-2.35 (m, 2 H) 3.05-3.22 (m, 2 H) 4.95-5.06 (m, 1 H) 7.50-7.62 (m, 5 H) 7.93 (d, J = 3 Hz, 1 H) 8.00-8.07 (m, 2 H) 8.13 (d, J = 3 Hz, 1 H) |
| 44 | 5-(2-methylcyclohexyl)-7- (phenylsulfonyl)-5H- pyrrolo[2,3-b]pyrazin-6- amine | 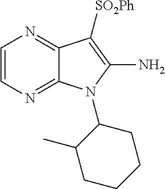 | 2-Methylcyclohexyl- amine, mixture of cis and trans (CAS 7003-32-9) | 1 | E | 371 | Mix of diastereoisomers in ~7:3 ratio (400 MHz, DMSO-d₆) 0.49-0.62 (m, 2.1 H) 0.77-0.84 (m, 0.9 H) 1.26-1.57 (m, 3 H) 1.65-1.75 (m, 2 H) 1.77-1.91 (m, 2 H) 2.31-2.47 (m, 1 H) 2.62-2.71 (m, 1 H) 4.00-4.12 (m, 0.7 H) 4.39-4.50 (m, 0.3 H) 7.52-7.69 (m, 5 H) 7.84-7.90 (m, 1 H) 7.99-8.15 (m, 3 H) |
| 45 | 5-butyl-7-5(phenylsulfonyl)- 5H-pyrrolo[2,3-b]pyrazin-6- amine | 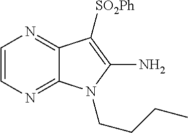 | Butan-1-amine (CAS 109-73-9) | 2 | D | 331 | (400 MHz, METHANOL-d₄) 0.95 (t, J = 7 Hz, 3 H) 1.30-1.42 (m, 2 H) 1.67-1.78 (m, 2 H) 4.17 (t, J = 7 Hz, 2 H) 7.47-7.61 (m, 3 H) 7.92 (d, J = 3 Hz, 1 H) 8.04-8.15 (m, 3 H) |
| 46 | 5-phenethyl-7- (phenylsulfonyl)-5H- pyrrolo[2,3-b]pyrazin-6- amine | 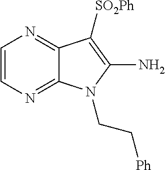 | 2-phenylethanamine (CAS 64-04-0) | 2 | E^{Note 1} | 379 | (400 MHz, METHANOL-d₄) 3.05 (t, J = 7 Hz, 2 H) 4.42 (t, J = 7 Hz, 2 H) 6.97-7.11 (m, 5 H) 7.50-7.64 (m, 3 H) 7.83 (d, J = 3 Hz, 1 H) 8.02 (d, J = 3 Hz, 1 H) 8.07 (d, J = 8 Hz, 2 H) |
| 47 | 2-(6-amino-7- (phenylsulfonyl)-5H- pyrrolo[2,3-b]pyrazin-5- yl)cyclohexanol | 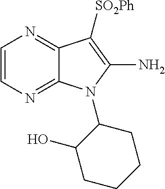 | 2-aminocyclohexanol (CAS 6850-38-0) | 3 | D | 373 | (400 MHz, DMSO-d₆) 1.32-1.51 (m, 3 H) 1.61-1.73 (m, 2 H) 1.76-1.88 (m, 2 H) 4.09-4.17 (m, 1 H) 4.86 (d, J = 13 Hz, 1 H) 7.50-7.64 (m, 3 H) 7.89 (d, J = 3 Hz, 1 H) 8.00-8.09 (m, 2 H) 8.12 (d, J = 3 Hz, 1 H), further 1 H multiplet obscured by DMSO |
| 48 | 5-(2-cyclopropylethyl)-7- (phenylsulfonyl)-5H- pyrrolo[2,3-b]pyrazin-6- amine | 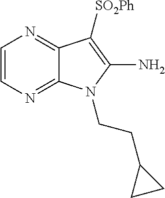 | 2-cyclopropyl- ethanamine (CAS 62893-54-3) | 3 | D | 343 | (400 MHz, DMSO-d₆) −0.19-−0.12 (m, 2 H) 0.16-0.24 (m, 2 H) 0.55-0.67 (m, 1 H) 1.47-1.58 (m, 2 H) 4.15-4.25 (m, 2 H) 7.49-7.71 (m, 5 H) 7.88 (d, J = 3 Hz, 1 H) 7.99-8.06 (m, 2 H) 8.09 (d, J = 3 Hz, 1 H) |
| 49 | 5-(4,4-difluoro-cyclohexyl)- 7-(phenylsulfonyl)-5H- pyrrolo[2,3-b]pyrazin-6- amine | 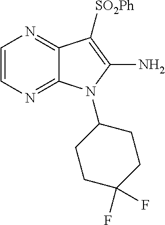 | 4,4-difluoro- cyclohexanamine hydrochloride (CAS 675112-70-6) | 3 | C | 393 | (400 MHz, DICHLOROMETHANE-d₂) 1.88-2.11 (m, 4 H) 2.25-2.39 (m, 2 H) 2.76-2.94 (m, 2 H) 4.29-4.45 (m, 1 H) 6.21 (br. s., 2 H) 7.47-7.63 (m, 3 H) 7.97 (d, J = 3 Hz, 1 H) 8.18 (d, J = 7 Hz, 2 H) 8.24 (d, J = 3 Hz, 1 H) |
| 50 | 5-(2-cyclobutylethyl)-7- (phenylsulfonyl)-5H- pyrrolo[2,3-b]pyrazin-6- amine | 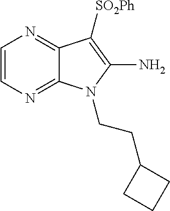 | 2-cyclobutyl- ethanamine (CAS 60637-97-0) | 3^{Note 2} | E | 357 | (400 MHz, DMSO-d₆) 1.46-1.58 (m, 2 H) 1.65-1.79 (m, 4 H) 1.80-1.91 (m, 2 H) 2.13-2.24 (m, 1 H) 4.05 (t, J = 7 Hz, 2 H) 7.51-7.71 (m, 5 H) 7.90 (d, J = 3 Hz, 1 H) 8.03 (d, J = 7 Hz, 2 H) 8.11 (d, J = 3 Hz, 1 H) |
| 51 | 7-(phenylsulfonyl)-5- (tetrahydro-2H-pyran-3-yl)- 5H-pyrrolo[2,3-b]pyrazin-6- amine | 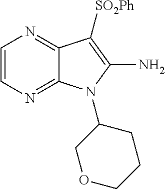 | tetrahydro-2H-pyran- 3-amine hydrochloride (CAS 675112-58-0) | 3^{Note 2} | E | 359 | (400 MHz, DMSO-d₆) 1.69-1.80 (m, 2 H) 1.84-1.94 (m, 1 H) 2.56-2.70 (m, 1 H) 3.36-3.45 (m, 1 H) 3.79-3.91 (m, 2 H) 4.26 (t, J = 10 Hz, 1 H) 4.52-4.62 (m, 1 H) 7.51-7.63 (m, 3 H) 7.71 (br. s., 2 H) 7.90 (d, J = 3 Hz, 1 H) 8.05 (d, J = 7 Hz, 2 H) 8.11 (d, J = 3 Hz, 1 H) |
| 52 | 5-(3,3-dimethylbutyl)-7- (phenylsulfonyl)-5H- pyrrolo[2,3-b]pyrazin-6- amine | 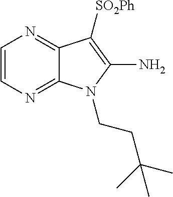 | 3,3-dimethylbutan-1- amine (CAS 15673-00-4) | 3 | E | 359 | (400 MHz, DMSO-d₆) 0.95 (s, 9 H) 1.45-1.56 (m, 2 H) 4.09-4.19 (m, 2 H) 7.50-7.66 (m, 5 H) 7.92 (d, J = 3 Hz, 1 H) 8.01-8.07 (m, 2 H) 8.11 (d, J = 3 Hz, 1 H) |
| 53 | 5-((1R*,2R*,4S*)- bicyclo[2.2.1]heptan-2-yl)-7- (phenylsulfonyl)-5H- pyrrolo[2,3-b)pyrazin-6- amine | 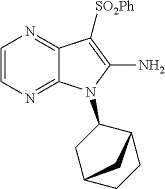 | exo-2- aminonorbornane (CAS 7242-92-4, Sigma-Aldrich cat. no. 179604) | 3 | E | 369 | (400 MHz, DMSO-d₆) 1.14-1.29 (m, 2 H) 1.43-1.59 (m, 3 H) 1.80-1.91 (m, 1 H) 2.29-2.42 (m, 2 H) 2.43-2.48 (m, 1 H) 2.60-2.67 (m, 1 H) 4.22-4.31 (m, 1 H) 7.45-7.64 (m, 5 H) 7.86 (d, J = 3 Hz, 1 H) 8.00-8.11 (m, 3 H) |
| 54 | 5-(cyclopentylmethyl)-7- (phenylsulfonyl)-5H- pyrrolo[2,3-b]pyrazin-6- amine | 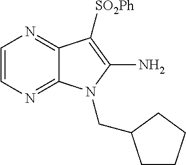 | Cyclopentyl- methanamine (CAS 6053-81-2) | 3 | E | 357 | (400 MHz, DMSO-d₆) 1.17-1.32 (m, 2 H) 1.39-1.69 (m, 6 H) 2.35-2.45 (m, 1 H) 4.08 (d, J = 8 Hz, 2 H) 7.51-7.61 (m, 3 H) 7.65 (br. s, 2 H) 7.89 (d, J = 3 Hz, 1 H) 8.01-8.07 (m, 2 H) 8.10 (d, J = 3 Hz, 1 H) |
| 55 | 5-((1-ethylcyclopropyl)- methyl)-7-(phenylsulfonyl)- 5H-pyrrolo[2,3-b]pyrazin-6- amine | 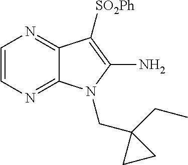 | (1-ethylcyclopropyl) methanamine (CAS 1177326-74-7) | 3 | E | 357 | (400 MHz, DMSO-d₆) 0.27-0.34 (m, 2 H) 0.51-0.58 (m, 2 H) 0.79 (t, J = 7 Hz, 3 H) 1.26 (q, J = 7 Hz, 2 H) 4.17 (s, 2 H) 7.46-7.62 (m, 5 H) 7.91 (d, J = 3 Hz, 1 H) 7.99-8.07 (m, 2 H) 8.11 (d, J = 3 Hz, 1 H) |
| 56 | 5-((2,2- dimethylcyclopropyl)methyl)- 7-(phenylsulfonyl)-5H- pyrrolo[2,3-b]pyrazin-6- amine | 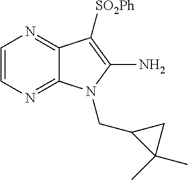 | (2,2- dimethylcyclopropyl) methanamine (CAS 725743-45-3) | 3 | E | 357 | (400 MHz, DMSO-d₆) 0.30-0.40 (m, 2 H) 1.00 (s, 3 H) 1.02-1.11 (m, 1 H) 1.19 (s, 3 H) 3.94-4.05 (m, 1 H) 4.28-4.38 (m, 1 H) 7.51-7.70 (m, 5 H) 7.90 (d, J = 3.03 Hz, 1 H) 8.02-8.08 (m, 2 H) 8.11 (d, J = 3.03 Hz, 1 H) |

^{Note 1}Followed by flash chromatography (12 g silica, 25-60% EtOAc/petrol)
^{Note 2}Aqueous workup

Examples 57 to 107 (see Table 3 following) were prepared according to one of the procedures 4 or 5 as described below.

### Procedure 4

To a solution of 5-cyclohexyl-6-(1,3-dioxo-2,3-dihydro-1H-isoindol-2-yl)-5H-pyrrolo[2,3-b]pyrazine-7-sulfonyl chloride (Intermediate 41; 50 mg, 0.112 mmol) in THF (1 mL) was added triethylamine (0.089 mL, 0.635 mmol) and a primary or secondary amine (0.175 mmol). The reaction was stirred at rt for 3 hours and then ethanol (1 mL) and hydrazine monohydrate (0.635 mmol) were added. The reaction mixture was warmed to 80° C. and maintained at this temperature overnight. The reaction mixtures were filtered and concentrated. The residue was taken up in DCM and washed with water, then the organic phase was separated and concentrated and the resulting crude product was purified via prep HPLC using one of the methods listed below or column chromatography on silica.

### Procedure 5

To a solution of 5-cyclohexyl-6-(1,3-dioxo-2,3-dihydro-1H-isoindol-2-yl)-5H-pyrrolo[2,3-b]pyrazine-7-sulfonyl chloride (Intermediate 41; 55 mg, 0.124 mmol) in THF (1 mL) was added triethylamine (0.052 mL, 0.371 mmol) and a primary or secondary amine (00.247 mmol). After 2 h at room temperature the mixture was diluted with water and extracted with DCM. The organic phase was concentrated, then ethanol (1 mL) and hydrazine monohydrate (0.018 mL, 0.371 mmol) were added and the reaction mixture was warmed to 70° C. for 3 h. The reaction mixture was filtered and concentrated and the residue was purified by column chromatography (silica, 0-100% EtOAc/petroleum ether).

| HPLC | Gradient (acetonitrile/water |
|---|---|
| Method | (with 0.1% ammonia)) |
| A | 5-25% |
| B | 5-40% |
| C | 10-50% |
| D | 20-60% |
| E | 30-70% |
| F | 40-80% |
| G | 55-95% |

TABLE 3

| | | | | Syn- | | | |
|---|---|---|---|---|---|---|---|
| | | | | thesis | | | |
| Ex. | | | | meth- | Purification | MS | ¹H NMR data |
| No. | Name of compound | Structure | Starting amine | od | method | ES+ | δ ppm |
| 57 | 5-cyclohexyl-7- (piperidin-1- ylsulfonyl)-5H- pyrrolo[2,3-b]pyrazin- 6-amine | 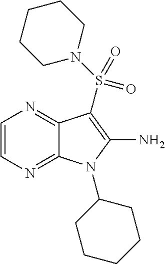 [SMILES: Nc1c(S(=O)(=O)N2CCCCC2)c2nccnc2n1C1CCCCC1] | Piperidine (CAS 110-89- 4) | 2 | 0-100% ethyl acetate/petrol | 364 | (400 MHz, DICHLOROMETHANE- d₂) 1.32-1.57 (m, 5 H) 1.59-1.71 (m, 4 H) 1.74-1.84 (m, 1 H) 1.87-2.07 (m, 4 H) 2.38-2.55 (m, 2 H) 3.08-3.29 (m, 4 H) 4.19-4.39 (m, 1 H) 5.97 (br. s., 2 H) 7.89-7.98 (m, 1 H) 8.14-8.23 (m, 1 H) |
| 58 | 5-cyclohexyl-7- (pyrrolidin-1- ylsulfonyl)-5H- pyrrolo[2,3-b]pyrazin- 6-amine | 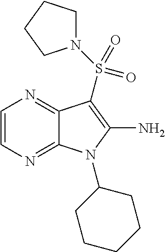 [SMILES: Nc1c(S(=O)(=O)N2CCCC2)c2nccnc2n1C1CCCCC1] | Pyrrolidine (CAS 123-75- 1) | 1 | 0-100% ethyl acetate/petrol | 350 | (400 MHz, DICHLOROMETHANE- d₂) 1.29-1.57 (m, 4 H) 1.73-1.84 (m, 4 H) 1.87-2.04 (m, 4 H) 2.37-2.56 (m, 2 H) 3.39-3.52 (m, 4 H) 4.23-4.38 (m, 1 H) 6.01 (br. s., 2 H) 7.94 (d, J = 3 Hz, 1 H) 8.18 (d, J = 3 Hz, 1 H) |
| 59 | 6-amino-5-cyclohexyl- N-propyl-5H- pyrrolo[2,3- b]pyrazine-7- sulfonamide | 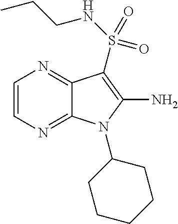 [SMILES: CCCNS(=O)(=O)c1c(N)n(C2CCCCC2)c2nccnc12] | Propan-1- amine (CAS 107-10- 8) | 1 | 0-100% ethyl acetate/petrol | 338 | (400 MHz, DMSO-d₆) 0.74 (t, J = 7 Hz, 3 H) 1.18-1.47 (m, 5 H) 1.66-1.76 (m, 3 H) 1.80-1.89 (m, 2 H) 2.42-2.57 (m, 2 H) 2.70-2.79 (m, 2 H) 4.31-4.44 (m, 1 H) 7.14 (t, J = 6 Hz, 1 H) 7.21 (s, 2 H) 7.88 (d, J = 3 Hz, 1 H) 8.08 (d, J = 3 Hz, 1 H) |
| 60 | 6-amino-5-cyclohexyl- N-methyl-N-propyl- 5H-pyrrolo[2,3- b]pyrazine-7- sulfonamide | 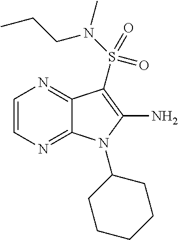 [SMILES: CCCN(C)S(=O)(=O)c1c(N)n(C2CCCCC2)c2nccnc12] | N- methylpropan- 1-amine (CAS 627-35- 0) | 1 | 0-100% ethyl acetate/petrol | 352 | (400 MHz, DICHLOROMETHANE- d₂) 0.94 (t, J = 7 Hz, 3 H) 1.33-1.65 (m, 5 H) 1.76-1.84 (m, 1 H) 1.88-2.06 (m, 4 H) 2.38-2.51 (m, 2 H) 2.84 (s, 3 H) 3.09-3.17 (m, 2 H) 4.23-4.36 (m, 1 H) 6.00 (br. s., 2 H) 7.95 (d, J = 3 Hz, 1 H) 8.18 (d, J = 3 Hz, 1 H) |
| 61 | 5-cyclohexyl-7- (morpholinosulfonyl)- 5H-pyrrolo[2,3- b]pyrazin-6-amine | 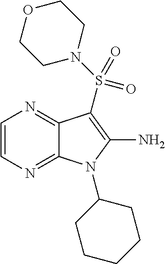 [SMILES: Nc1c(S(=O)(=O)N2CCOCC2)c2nccnc2n1C1CCCCC1] | Morpholine (CAS 110-91- 8) | 1 | 0-100% ethyl acetate/petrol | 366 | (400 MHz, DICHLOROMETHANE- d₂) 1.33-1.59 (m, 3 H) 1.77-1.85 (m, 1 H) 1.88-2.06 (m, 4 H) 2.38-2.57 (m, 2 H) 3.14-3.27 (m, 4 H) 3.70-3.81 (m, 4 H) 4.22-4.40 (m, 1 H) 6.02 (br. s., 2 H) 7.97 (d, J = 3 Hz, 1 H) 8.19 (d, J = 3 Hz, 1 H) |
| 62 | 5-cyclohexyl-7-((4- methylpiperidin-1- yl)sulfonyl)-5H- pyrrolo[2,3-b]pyrazin- 6-amine | 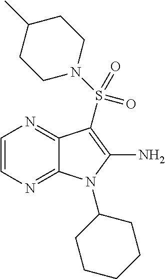 [SMILES: CC1CCN(S(=O)(=O)c2c(N)n(C3CCCCC3)c3nccnc23)CC1] | 4-methyl- piperidine (CAS 626-58- 4) | 1 | 0-100% ethyl acetate/petrol | 378 | (400 MHz, DICHLOROMETHANE- d₂) 0.81-0.97 (m, 3 H) 1.24-1.57 (m, 6 H) 1.64-1.73 (m, 2 H) 1.76-1.84 (m, 1 H) 1.89-2.05 (m, 4 H) 2.38-2.50 (m, 2 H) 2.52-2.62 (m, 2 H) 3.76-3.85 (m, 2 H) 4.20-4.39 (m, 1 H) 6.00 (br. s., 2 H) 7.95 (d, J = 3 Hz, 1 H) 8.18 (d, J = 3 Hz, 1 H) |
| 63 | 5-cyclohexyl-7-((4- methylpiperazin-1- yl)sulfonyl)-5H- pyrrolo[2,3-b]pyrazin- 6-amine | 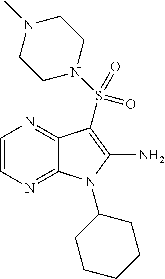 [SMILES: CN1CCN(S(=O)(=O)c2c(N)n(C3CCCCC3)c3nccnc23)CC1] | 1-methyl- piperazine (CAS 109-01- 3) | 1 | 0-100% ethyl acetate/petrol | 379 | (400 MHz, DICHLOROMETHANE- d₂) 1.35-1.57 (m, 3 H) 1.76-1.85 (m, 1 H) 1.89-2.03 (m, 4 H) 2.25 (s, 3 H) 2.37-2.57 (m, 6 H) 3.17-3.30 (m, 4 H) 4.22-4.34 (m, 1 H) 5.92 (br. s., 2H) 7.95 (d, J = 3 Hz, 1 H) 8.18 (d, J = 3 Hz, 1 H) |
| 64 | 5-cyclohexyl-7-((3- methoxyazetidin-1- yl)sulfonyl)-5H- pyrrolo[2,3-b]pyrazin- 6-amine | 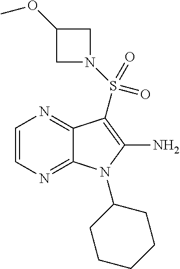 [SMILES: COC1CN(S(=O)(=O)c2c(N)n(C3CCCCC3)c3nccnc23)C1] | 3-metboxy- azetidine (CAS 110925- 17-2) | 1 | 0-100% ethyl acetate/petrol | 366 | (400 MHz, DICHLOROMETHANE- d₂) 1.33-1.58 (m, 3 H) 1.76-1.84 (m, 1 H) 1.90-2.04 (m, 4 H) 2.40-2.55 (m, 2 H) 3.14 (s, 3 H) 3.82-3.90 (m, 2 H) 4.00-4.06 (m, 1 H) 4.07-4.14 (m, 2 H) 4.25-4.38 (m, 1 H) 6.09 (br. s., 2 H) 7.99 (d, J = 3 Hz, 1 H) 8.22 (d, J = 3 Hz, 1 H) |
| 65 | 5-cyclohexyl-7-((4- ethoxypiperidin-1- yl)sulfonyl)-5H- pyrrolo[2,3-b]pyrazin- 6-amine | 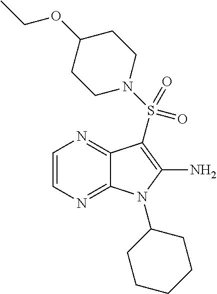 [SMILES: CCOC1CCN(S(=O)(=O)c2c(N)n(C3CCCCC3)c3nccnc23)CC1] | 4-ethoxy- piperidine (CAS 1122-86- 7) | 1 | 0-100% ethyl acetate/petrol | 408 | (400 MHz, DICHLOROMETHANE- d₂) 1.05-1.15 (m, 3 H) 1.26-1.51 (m, 3 H) 1.57-1.66 (m, 2 H) 1.75-1.83 (m, 1 H) 1.84-1.98 (m, 6 H) 2.38-2.53 (m, 2 H) 2.96-3.05 (m, 2 H) 3.28-3.37 (m, 1 H) 3.37-3.48 (m, 4 H) 4.26 (br. s., 1 H) 6.01 (br. s., 2 H) 7.92 (d, J = 3 Hz, 1 H) 8.14 (d, J = 3 Hz, 1 H) |
| 66 | 5-cyclohexyl-7-((4,4- dimethylpiperidin-1- yl)sulfonyl)-5H- pyrrolo[2,3-b]pyrazin- 6-amine | 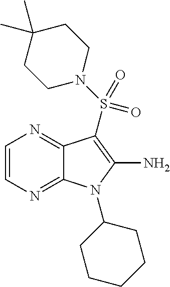 [SMILES: CC1(C)CCN(S(=O)(=O)c2c(N)n(C3CCCCC3)c3nccnc23)CC1] | 4,4-dimethyl- piperidine hydrochloride (CAS 38646- 68-3) | 1 | 0-100% ethyl acetate/petrol | 392 | (400 MHz, DICHLOROMETHANE- d₂) 0.88 (s, 6 H) 1.32-1.56 (m, 7 H) 1.76-1.84 (m, 1 H) 1.89-2.03 (m, 4 H) 2.39-2.53 (m, 2 H) 3.16-3.26 (m, 4 H) 4.23-4.36 (m, 1 H) 5.99 (br. s., 2 H) 7.95 (d, J = 3 Hz, 1 H) 8.19 (d, J = 3 Hz, 1 H) |
| 67 | 5-cyclohexyl-7-((3- methylpyrrolidin-1- yl)sulfonyl)-5H- pyrrolo[2,3-b]pyrazin- 6-amine | 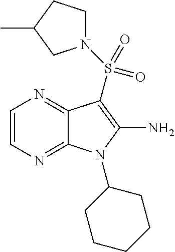 [SMILES: CC1CCN(S(=O)(=O)c2c(N)n(C3CCCCC3)c3nccnc23)C1] | 3-methyl- pyrrolidine (CAS 34375- 89-8) | 1 | 0-100% ethyl acetate/petrol | 364 | (400 MHz, DICHLOROMETHANE- d₂) 0.95 (d, J = 7 Hz, 3 H) 1.34-1.57 (m, 4 H) 1.77-1.85 (m, 1 H) 1.88-2.04 (m, 5 H) 2.08-2.22 (m, 1 H) 2.39-2.54 (m, 2 H) 2.90-2.98 (m, 1 H) 3.39-3.49 (m, 1 H) 3.59-3.73 (m, 2 H) 4.24-4.39 (m, 1 H) 6.05 (br. s., 2 H) 7.95 (d, J = 3 Hz, 1 H) 8.18 (d, J = 3 Hz, 1H) |
| 68 | 5-cyclohexyl-7-((2- methylpyrrolidin-1- yl)sulfonyl)-5H- pyrrolo[2,3-b]pyrazin- 6-amine | 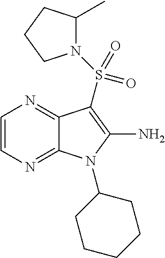 [SMILES: CC1CCCN1S(=O)(=O)c1c(N)n(C2CCCCC2)c2nccnc12] | 2-methyl- pyrrolidine (CAS) | 1 | 0-100% ethyl acetate/petrol | 364 | (400 MHz, DICHLOROMETHANE- d₂) 1.32 (d, J = 7 Hz, 3 H) 1.35-1.63 (m, 5 H) 1.69-1.87 (m, 3 H) 1.89-2.04 (m, 4 H) 2.37-2.51 (m, 2 H) 3.39-3.55 (m, 2 H) 4.18-4.36 (m, 2 H) 6.03 (br. s., 2 H) 7.94 (d, J = 3 Hz, 1 H) 8.18 (d, J = 3 Hz, 1 H) |
| 69 | 5-cyclohexyl-7-((4,4- difluoropiperidin-1- yl)sulfonyl)-5H- pyrrolo[2,3-b]pyrazin- 6-amine | 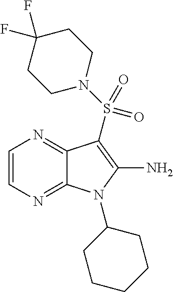 [SMILES: Nc1c(S(=O)(=O)N2CCC(F)(F)CC2)c2nccnc2n1C1CCCCC1] | 4,4-difluoro- piperidine (CAS 21987- 29-1) | 1 | 0-100% ethyl acetate/petrol | 400 | (400 MHz, DICHLOROMETHANE- d₂) 1.34-1.56 (m, 3 H) 1.77-1.84 (m, 1 H) 1.89-2.02 (m, 4 H) 2.04-2.17 (m, 4 H) 2.39-2.54 (m, 2 H) 3.37-3.48 (m, 4 H) 4.21-4.38 (m, 1 H) 6.02 (br. s., 2 H) 7.93-8.01 (m, 1 H) 8.15-8.23 (m, 1 H) |
| 70 | 6-amino-N-benzyl-5- cyclohexyl-5H- pyrrolo[2,3- b]pyrazine-7- sulfonamide | 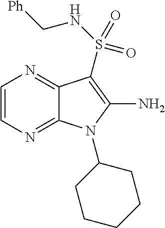 [SMILES: Nc1c(S(=O)(=O)NCc2ccccc2)c2nccnc2n1C1CCCCC1] | Benzylamine (CAS 100-46- 9) | 1 | 0-100% ethyl acetate/petrol | 386 | (400 MHz, DICHLOROMETHANE- d₂) 1.33-1.55 (m, 3 H) 1.75-1.85 (m, 1 H) 1.86-2.06 (m, 4 H) 2.33-2.51 (m, 2 H) 4.10 (d, J = 7 Hz, 2 H) 4.18-4.28 (m, 1 H) 5.51 (t, J = 6 Hz, 1 H) 5.87 (br. s., 2 H) 7.12-7.22 (m, 5 H) 7.95 (d, J = 3 Hz, 1 H) 8.15 (d, J = 3 Hz, 1 H) |
| 71 | 6-amino-N,5- dicyclohexyl-N- methyl-5H- pyrrolo[2,3- b]pyrazine-7- sulfonamide | 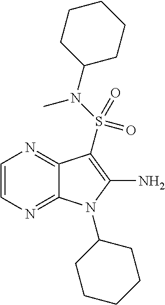 [SMILES: CN(C1CCCCC1)S(=O)(=O)c1c(N)n(C2CCCCC2)c2nccnc12] | N-methyl- cyclohexan- amine (CAS 100-60- 7) | 1 | 0-100% ethyl acetate/petrol | 392 | (400 MHz, DICHLOROMETHANE- d₂) 1.00-1.11 (m, 1 H) 1.27-1.65 (m, 10 H) 1.68-1.83 (m, 3 H) 1.86-2.05 (m, 4 H) 2.38-2.54 (m, 2 H) 2.83 (s, 3 H) 3.83-3.94 (m, 1 H) 4.21-4.35 (m, 1 H) 5.97 (br. s., 2 H) 7.93 (d, J = 3 Hz, 1 H) 8.19 (d, J = 3 Hz, 1 H) |
| 72 | 5-cyclohexyl-7-(1,4- oxazepane-4-sulfonyl)- 5H-pyrrolo[2,3- b]pyrazin-6-amine | 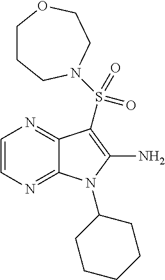 [SMILES: Nc1c(S(=O)(=O)N2CCCOCC2)c2nccnc2n1C1CCCCC1] | 1,4-oxazepane hydrochloride (CAS 178312- 62-4) | 1 | 0-100% ethyl acetate/petrol | 380 | (400 MHz, DICHLOROMETHANE- d₂) 1.33-1.55 (m, 3 H) 1.76-1.86 (m, 1 H) 1.89-2.04 (m, 6 H) 2.37-2.53 (m, 2 H) 3.51-3.61 (m, 4 H) 3.71-3.80 (m, 4 H) 4.20-4.36 (m, 1 H) 5.94 (br. s., 2 H) 7.95 (d, J = 3 Hz, 1 H) 8.18 (d, J = 3 Hz, 1 H) |
| 73 | 5-cyclohexyl-7-(4- methoxypiperidine-1- sulfonyl)-5H- pyrrolo[2,3-b]pyrazin- 6-amine | 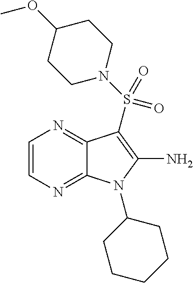 [SMILES: COC1CCN(S(=O)(=O)c2c(N)n(C3CCCCC3)c3nccnc23)CC1] | 4-methoxy- piperidine (CAS 4045-24- 3) | 1 | 0-100% ethyl acetate/petrol | 394 | (400 MHz, DICHLOROMETHANE- d₂) 1.35-1.55 (m, 3 H) 1.64-1.73 (m, 2 H) 1.77-1.84 (m, 1 H) 1.87-2.04 (m, 6 H) 2.39-2.54 (m, 2 H) 3.05-3.13 (m, 2 H) 3.18-3.30 (m, 4 H) 3.36-3.45 (m, 2 H) 4.20-4.38 (m, 1 H) 5.98 (br. s., 2 H) 7.96 (d, J = 3 Hz, 1 H) 8.19 (d, J = 3 Hz, 1 H) |
| 74 | 6-amino-N- (cyclobutylmethyl)-5- cyclohexyl-5H- pyrrolo[2,3- b]pyrazine-7- sulfonamide | 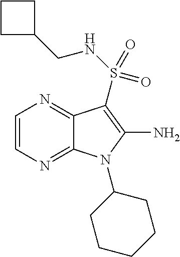 [SMILES: Nc1c(S(=O)(=O)NCC2CCC2)c2nccnc2n1C1CCCCC1] | Cyclobutyl- methanamine hydrochloride (CAS 5454-82- 0) | 1 | 0-100% ethyl acetate/petrol | 364 | (400 MHz, DICHLOROMETHANE- d₂) 1.35-1.55 (m, 3 H) 1.57-1.68 (m, 2 H) 1.75-2.05 (m, 9 H) 2.39-2.54 (m, 3 H) 2.82-2.91 (m, 2 H) 4.21-4.34 (m, 1 H) 5.04 (t, J = 6 Hz, 1 H) 5.88 (br. s., 2 H) 7.98 (d, J = 3 Hz, 1 H) 8.18 (d, J = 3 Hz, 1 H) |
| 75 | 5-cyclohexyl-7-(3,3- dimethylpyrrrolidine- 1-sulfonyl)-5H- pyrrolo[2,3-b]pyrazin- 6-amine | 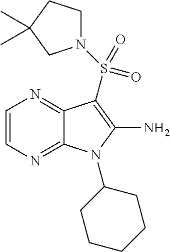 [SMILES: CC1(C)CCN(S(=O)(=O)c2c(N)n(C3CCCCC3)c3nccnc23)C1] | 3,3-Dimethyl- pyrrolidine (CAS 3437-30- 7) | 1 | 0-100% ethyl acetate/petrol | 378 | (400 MHz, DICHLOROMETHANE- d₂) 0.97 (s, 6 H) 1.33-1.53 (m, 3 H) 1.58 (t, J = 7 Hz, 2 H) 1.77-1.85 (m, 1 H) 1.90-2.04 (m, 4 H) 2.38-2.53 (m, 2 H) 3.18 (s, 2 H) 3.61 (t, J = 7 Hz, 2 H) 4.24-4.35 (m, 1 H) 5.96 (br. s., 2 H) 7.95 (d, J = 3 Hz, 1 H) 8.19 (d, J = 3 Hz, 1 H) |
| 76 | 5-cyclohexyl-7-(2,6- dimethylmorpholine-4- sulfonyl)-5H- pyrrolo[2,3-b]pyrazin- 6-amine | 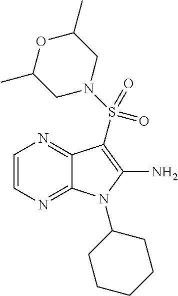 [SMILES: CC1CN(S(=O)(=O)c2c(N)n(C3CCCCC3)c3nccnc23)CC(C)O1] | 2,6-dimethyl- morpholine (mixture of stereoisomers; CAS 141-91-3) | 1 | 0-100% ethyl acetate/petrol | 394 | Mixture of diasteroisomers in ~4:1 ratio. (400 MHz, DICHLOROMETHANE- d₂) 1.11-1.16 (m, 4.5 H) 1.21-1.27 (m, 1.5 H) 1.35-1.58 (m, 3 H) 1.77-1.85 (m, 1 H) 1.89-2.07 (m, 4 H) 2.28-2.39 (m, 1.6 H) 2.41-2.53 (m, 2 H) 2.85-2.95 (m, 0.4 H) 3.20-3.28 (m, 0.4 H) 3.59-3.65 (m, 1.6 H) 3.67-3.75 (m, 1.6 H) 4.01-4.11 (m, 0.4 H) 4.23-4.36 (m, 1 H) 5.96 (br. s., 2 H) 7.92-8.01 (m, 1 H) 8.16-8.23 (m, 1 H) |
| 77 | 7-(azepane-1- sulfonyl)-5- cyclohexyl-5H- pyrrolo[2,3-b]pyrazin- 6-amine | 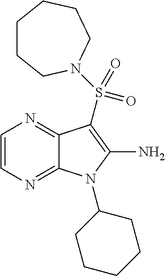 [SMILES: Nc1c(S(=O)(=O)N2CCCCCC2)c2nccnc2n1C1CCCCC1] | Azepane (CAS 111-49- 9) | 1 | 0-100% ethyl acetate/petrol | 378 | (400 MHz, DICHLOROMETHANE- d₂) 1.33-1.57 (m, 3 H) 1.58-1.64 (m, 4 H) 1.69-1.84 (m, 5 H) 1.88-2.04 (m, 4 H) 2.36-2.51 (m, 2 H) 3.37-3.46 (m, 4 H) 4.22-4.34 (m, 1 H) 5.92 (br. s., 2 H) 7.94 (d, J = 3 Hz, 1 H) 8.18 (d, J = 3 Hz, 1 H) |
| 78 | 5-cyclohexyl-7- (thiomorpholine-4- sulfonyl)-5H- pyrrolo[2,3-b]pyrazin- 6-amine | 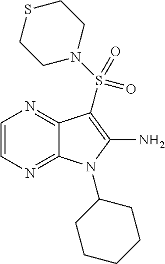 [SMILES: Nc1c(S(=O)(=O)N2CCSCC2)c2nccnc2n1C1CCCCC1] | Thio- morpholine (CAS 123-90- 0) | 1 | E | 382 | (400 MHz, DMSO-d₆) 1.21-1.33 (m, 1 H) 1.35-1.50 (m, 2 H) 1.64-1.78 (m, 3 H) 1.81-1.90 (m, 2 H) 2.43-2.56 (m, 2 H) 2.61-2.70 (m, 4 H) 3.32-3.40 (m, 4 H) 4.35-4.47 (m, 1 H) 7.32 (s, 2 H) 7.91 (d, J = 3 Hz, 1 H) 8.08 (d, J = 3 Hz, 1 H) |
| 79 | N-(1-{6-amino-5- cyclohexyl-5H- pyrrolo[2,3- b]pyrazine-7- sulfonyl}piperidin-4- yl)-N-methylacetamide | 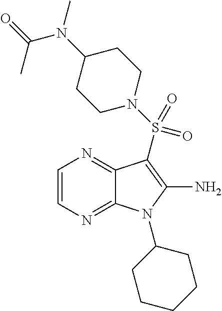 [SMILES: CC(=O)N(C)C1CCN(S(=O)(=O)c2c(N)n(C3CCCCC3)c3nccnc23)CC1] | N-methyl-N- (piperidin-4- yl)acetamide (CAS 83180- 55-6) | 1 | D | 435 | (400 MHz, DMSO-d₆) 1.24-1.33 (m, 1 H) 1.36-1.49 (m, 3 H) 1.56-1.79 (m, 6 H) 1.81-1.88 (m, 2 H) 1.90-1.97 (m, 3 H) 2.52-2.61 (m, 4 H) 2.63-2.72 (m, 2 H) 3.53-3.68 (m, 1 H) 3.75-3.85 (m, 2 H) 4.10-4.22 (m, 1 H) 4.36-4.47 (m, 1 H) 7.25-7.37 (m, 2 H) 7.86-7.94 (m, 1 H) 8.06-8.13 (m, 1 H) |
| 80 | 6-amino-5-cyclohexyl- N-(oxetan-3-ylmethyl)- 5H-pyrrolo[2,3- b]pyrazine-7- sulfonamide | 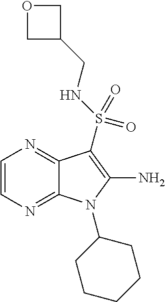 [SMILES: Nc1c(S(=O)(=O)NCC2COC2)c2nccnc2n1C1CCCCC1] | Oxetan-3-yl- methanamine (CAS 6246-05- 5) | 1 | D | 366 | (400 MHz, DMSO-d₆) 1.23-1.33 (m, 1 H) 1.35-1.50 (m, 2 H) 1.66-1.78 (m, 3 H) 1.82-1.90 (m, 2 H) 2.43-2.57 (m, 2 H) 2.89-3.03 (m, 1 H) 3.05-3.12 (m, 2 H) 4.12-4.19 (m, 2 H) 4.34-4.43 (m, 1 H) 4.45-4.51 (m, 2 H) 7.23 (s, 2 H) 7.38 (t, J = 6 Hz, 1 H) 7.90 (d, J = 3 Hz, 1 H) 8.09 (d, J = 3 Hz, 1 H) |
| 81 | 7-(4-benzylpiperidine- 1-sulfonyl)-5- cyclohexyl-5H- pyrrolo[2,3-b]pyrazin- 6-amine | 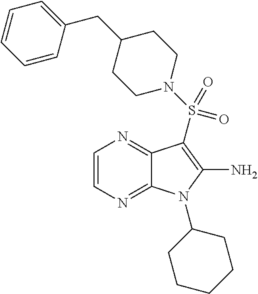 [SMILES: Nc1c(S(=O)(=O)N2CCC(Cc3ccccc3)CC2)c2nccnc2n1C1CCCCC1] | 4-benzyl- piperidine (CAS 31252- 42-3) | 1 | F | 454 | (400 MHz, DICHLOROMETHANE- d₂) 1.29-1.56 (m, 5 H) 1.67-1.73 (m, 2 H) 1.77-1.84 (m, 1 H) 1.87-1.94 (m, 2 H) 1.95-2.05 (m, 3 H) 2.37-2.59 (m, 6 H) 3.76-3.86 (m, 2 H) 4.22-4.34 (m, 1 H) 5.91 (s, 2 H) 7.08-7.15 (m, 2 H) 7.15-7.22 (m, 1 H) 7.24-7.31 (m, 2 H) 7.94 (d, J = 3 Hz, 1 H) 8.17 (d, J = 3 Hz, 1 H) |
| 82 | 6-amino-5-cyclohexyl- N-(3,3,3- trifluoropropyl)-5H- pyrrolo[2,3- b]pyrazine-7- sulfonamide | 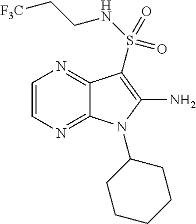 [SMILES: Nc1c(S(=O)(=O)NCCC(F)(F)F)c2nccnc2n1C1CCCCC1] | 3,3,3-trifluoro- propan-1- amine (CAS 460-39- 9) | 1 | E | 392 | (400 MHz, DMSO-d₆) 1.23-1.32 (m, 1 H) 1.34-1.49 (m, 2 H) 1.65-1.78 (m, 3 H) 1.81-1.90 (m, 2 H) 2.34-2.55 (m, 4 H) 3.05-3.13 (m, 2 H) 4.33-4.45 (m, 1 H) 7.26 (s, 2 H) 7.36-7.44 (m, 1 H) 7.90 (d, J = 3 Hz, 1 H) 8.08 (d, J = 3 Hz, 1 H) |
| 83 | 5-cyclohexyl-7-(4- phenylpiperidine-1- sulfonyl)-5H- pyrrolo[2,3-b]pyrazin- 6-amine | 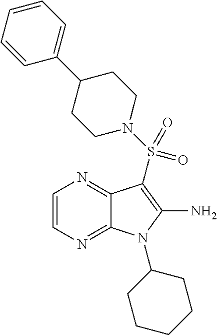 [SMILES: Nc1c(S(=O)(=O)N2CCC(c3ccccc3)CC2)c2nccnc2n1C1CCCCC1] | 4-phenyl- piperidine (CAS 771-99- 3) | 1 | F | 440 | (400 MHz, DICHLOROMETHANE- d₂) 1.34-1.58 (m, 3 H) 1.77-2.07 (m, 9 H) 2.39-2.56 (m, 3 H) 2.68-2.78 (m, 2 H) 3.92-4.07 (m, 2 H) 4.25-4.37 (m, 1 H) 5.96 (s, 2 H) 7.15-7.25 (m, 3 H) 7.28-7.34 (m, 2 H) 7.98 (d, J = 3 Hz, 1 H) 8.22 (d, J = 3 Hz, 1 H) |
| 84 | 6-amino-5-cyclohexyl- N-(2-phenylethyl)-5H- pyrrolo[2,3- b]pyrazine-7- sulfonamide | 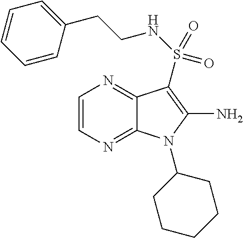 [SMILES: Nc1c(S(=O)(=O)NCCc2ccccc2)c2nccnc2n1C1CCCCC1] | 2-phenyl- ethanamine (CAS 64-04-0) | 1 | F | 400 | (400 MHz, DMSO-d₆) 1.21-1.32 (m, 1 H) 1.35-1.49 (m, 2 H) 1.64-1.77 (m, 3 H) 1.80-1.90 (m, 2 H) 2.43-2.55 (m, 2 H) 2.61-2.69 (m, 2 H) 3.01-3.09 (m, 2 H) 4.32-4.44 (m, 1 H) 7.04-7.16 (m, 3 H) 7.17-7.26 (m, 5 H) 7.88 (d, J = 3 Hz, 1 H) 8.06 (d, J = 3 Hz, 1 H) |
| 85 | 5-cyclohexyl-7-(4- phenoxypiperidine-1- sulfonyl)-5H- pyrrolo[2,3-b]pyrazin- 6-amine | 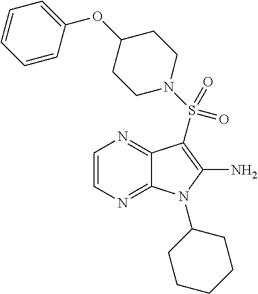 [SMILES: Nc1c(S(=O)(=O)N2CCC(Oc3ccccc3)CC2)c2nccnc2n1C1CCCCC1] | 4-phenoxy- piperidine (CAS 3202-33- 3) | 1 | F | 456 | (400 MHz, DMSO-d₆) 1.25-1.33 (m, 1 H) 1.36-1.50 (m, 2 H) 1.56-1.79 (m, 5 H) 1.83-1.90 (m, 2 H) 1.94-2.04 (m, 2 H) 2.46-2.58 (m, 2 H) 2.85-2.96 (m, 2 H) 3.44-3.53 (m, 2 H) 4.35-4.48 (m, 2 H) 6.82-6.91 (m, 3 H) 7.17-7.24 (m, 2 H) 7.31 (s, 2 H) 7.91 (d, J = 3 Hz, 1 H) 8.10 (d, J = 3 Hz, 1 H) |
| 86 | 5-cyclohexyl-7-(3- phenylpyrrolidine-1- sulfonyl)-5H- pyrrolo[2,3-b]pyrazin- 6-amine | 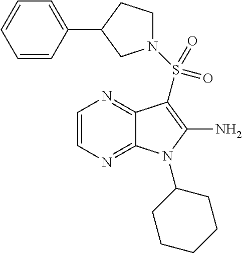 [SMILES: Nc1c(S(=O)(=O)N2CCC(c3ccccc3)C2)c2nccnc2n1C1CCCCC1] | 3-phenyl- pyrrolidine (CAS 936-44- 7) | 1 | F | 426 | (400 MHz, DMSO-d₆) 1.22-1.35 (m, 1 H) 1.35-1.51 (m, 2 H) 1.66-1.79 (m, 4 H) 1.82-1.93 (m, 2 H) 2.03-2.13 (m, 1 H) 2.46-2.57 (m, 2 H) 3.13-3.28 (m, 2 H) 3.33-3.43 (m, 1 H) 3.61-3.70 (m, 1 H) 3.89-3.98 (m, 1 H) 4.36-4.51 (m, 1 H) 7.04-7.13 (m, 2 H) 7.14-7.28 (m, 3 H) 7.31 (s, 2 H) 7.92 (d, J = 3 Hz, 1 H) 8.11 (d, J = 3 Hz, 1 H) |
| 87 | 5-cyclohexyl-7-[4- (trifluoromethyl) piperidine-1-sulfonyl]- 5H-pyrrolo[2,3- b]pyrazin-6-amine | 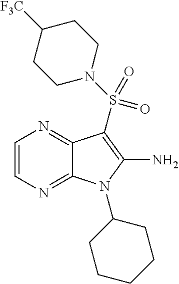 [SMILES: Nc1c(S(=O)(=O)N2CCC(C(F)(F)F)CC2)c2nccnc2n1C1CCCCC1] | 4-(trifluoro- methyl)- piperidine (CAS 657-36- 3) | 1 | F | 432 | (400 MHz, DICHLOROMETHANE- d₂) 1.34-1.54 (m, 3 H) 1.66-1.75 (m, 2 H) 1.77-1.86 (m, 1 H) 1.89-2.11 (m, 7 H) 2.39-2.52 (m, 2 H) 2.60-2.70 (m, 2 H) 3.93-4.03 (m, 2 H) 4.24-4.35 (m, 1 H) 5.94 (br. s., 2 H) 7.97 (d, J = 3 Hz, 1 H) 8.19 (d, J = 3 Hz, 1 H) |
| 88 | 5-cyclohexyl-7-[3- (methoxyrnethyl) pyrrolidine-1- sulfonyl]-5H- pyrrolo[2,3-b]pyrazin- 6-amine | 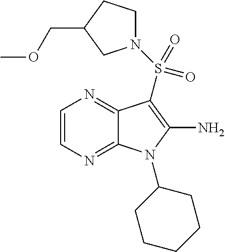 [SMILES: COCC1CCN(S(=O)(=O)c2c(N)n(C3CCCCC3)c3nccnc23)C1] | 3-(methoxy- methyl)- pyrrolidine (CAS 936940- 38-4) | 1 | E | 394 | (400 MHz, DMSO-d₆) 1.24-1.33 (m, 1 H) 1.36-1.50 (m, 3 H) 1.65-1.80 (m, 4 H) 1.81-1.90 (m, 2 H) 2.20-2.30 (m, 1 H) 2.43-2.56 (m, 2 H) 2.95-3.13 (m, 6 H) 3.23-3.29 (m, 1 H) 3.38-3.50 (m, 2 H) 4.34-4.45 (m, 1 H) 7.29 (s, 2 H) 7.90 (d, J = 3 Hz, 1 H) 8.08 (d, J = 3 Hz, 1 H) |
| 89 | 6-amino-5-cyclohexyl- N- (cyclopropylmethyl)- 5H-pyrrolo[2,3- b]pyrazine-7- sulfonamide | 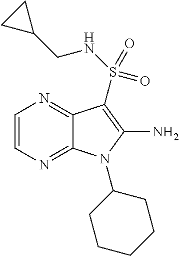 [SMILES: Nc1c(S(=O)(=O)NCC2CC2)c2nccnc2n1C1CCCCC1] | Cyclopropyl- methanamine (CAS 2516-47- 4) | 1 | E | 350 | (400 MHz, DMSO-d₆) −0.03-0.05 (m, 2 H) 0.20-0.29 (m, 2 H) 0.72-0.80 (m, 1 H) 1.22-1.31 (m, 1 H) 1.34-1.47 (m, 2 H) 1.62-1.74 (m, 3 H) 1.78-1.90 (m, 2 H) 2.40-2.52 (m, 2 H) 2.67-2.73 (m, 2 H) 4.31-4.43 (m, 1 H) 7.11-7.24 (m, 3 H) 7.86 (d, J = 3 Hz, 1 H) 8.06 (d, J = 3 Hz, 1 H) |
| 90 | 6-amino-5-cyclohexyl- N-(2-methoxyethyl)- 5H-pyrrolo[2,3- b]pyrazine-7- sulfonamide | 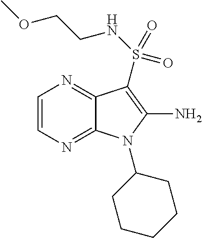 [SMILES: COCCNS(=O)(=O)c1c(N)n(C2CCCCC2)c2nccnc12] | 2-methoxy- ethanamine (CAS 109-85- 3) | 1 | D | 354 | (400 MHz, DMSO-d₆) 1.24-1.34 (m, 1 H) 1.36-1.49 (m, 2 H) 1.66-1.78 (m, 3 H) 1.81-1.90 (m, 2 H) 2.44-2.56 (m, 2 H) 2.95-3.03 (m, 2 H) 3.11 (s, 3 H) 3.25-3.30 (m, 2 H) 4.34-4.44 (m, 1 H) 7.06-7.13 (m, 1 H) 7.21 (s, 2 H) 7.89 (d, J = 3 Hz, 1 H) 8.08 (d, J = 3 Hz, 1 H) |
| 91 | 5-cyclohexyl-7-(3- methoxypyrrolidine-1- sulfonyl)-5H- pyrrolo[2,3-b]pyrazin- 6-amine | 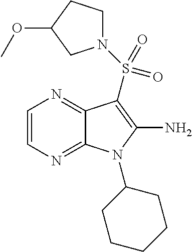 [SMILES: COC1CCN(S(=O)(=O)c2c(N)n(C3CCCCC3)c3nccnc23)C1] | 3-methoxy- pyrrolidine (CAS 62848- 20-8) | 1 | E | 380 | (400 MHz, DICHLOROMETHANE- d₂) 1.34-1.44 (m, 1 H) 1.46-1.60 (m, 2 H) 1.78-1.84 (m, 1 H) 1.86-2.03 (m, 6 H) 2.39-2.55 (m, 2 H) 3.14 (s, 3 H) 3.45-3.63 (m, 4 H) 3.83-3.91 (m, 1 H) 4.31-4.44 (m, 1 H) 6.22-6.45 (m, 2 H) 7.97 (d, J = 3 Hz, 1 H) 8.18 (d, J = 3 Hz, 1 H) |
| 92 | 5-cyclohexyl-7-(3,3- dimethylpiperidine-1- sulfonyl)-5H- pyrrolo[2,3-b]pyrazin- 6-amine | 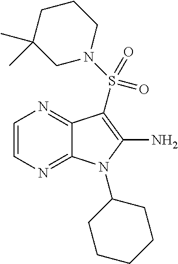 [SMILES: CC1(C)CCCN(S(=O)(=O)c2c(N)n(C3CCCCC3)c3nccnc23)C1] | 3,3-dimethyl- piperidine (CAS 1193-12- 0) | 1 | F | 392 | (400 MHz, DICHLOROMETHANE- d₂) 0.99 (s, 6 H) 1.22-1.28 (m, 2 H) 1.35-1.55 (m, 3 H) 1.66-1.73 (m, 2 H) 1.77-1.86 (m, 1 H) 1.89-2.04 (m, 4 H) 2.37-2.54 (m, 2 H) 2.82 (s, 2 H) 3.09-3.19 (m, 2 H) 4.19-4.35 (m, 1 H) 5.91 (br. s., 2 H) 7.95 (d, J = 3 Hz, 1 H) 8.19 (d, J = 3 Hz, 1 H) |
| 93 | 1-{6-amino-5- cyclohexyl-5H- pyrrolo[2,3- b]pyrazine-7- sulfonyl}piperidin-4-ol | 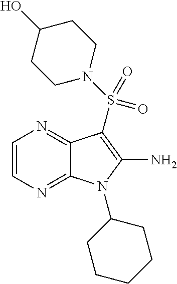 [SMILES: Nc1c(S(=O)(=O)N2CCC(O)CC2)c2nccnc2n1C1CCCCC1] | piperidin-4-ol (CAS 5382-16- 1) | 1 | D | 380 | (400 MHz, DMSO-d₆) 1.22-1.33 (m, 1 H) 1.35-1.48 (m, 4 H) 1.68-1.78 (m, 5 H) 1.80-1.91 (m, 2 H) 2.43-2.56 (m, 2 H) 2.77-2.87 (m, 2 H) 3.27-3.37 (m, 2 H) 3.47-3.56 (m, 1 H) 4.34-4.45 (m, 1 H) 4.59 (d, J = 4 Hz, 1 H) 7.27 (s, 2 H) 7.89 (d, J = 3 Hz, 1 H) 8.08 (d, J = 3 Hz, 1 H) |
| 94 | 5-cyclohexyl-7- (1,2,3,4- tetrahydroisoquinoline- 2-sulfonyl)-5H- pyrrolo[2,3-b]pyrazin- 6-amine | 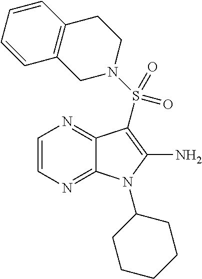 [SMILES: Nc1c(S(=O)(=O)N2CCc3ccccc3C2)c2nccnc2n1C1CCCCC1] | 1,2,3,4- tetrahydro- isoquinoline (CAS 91-21-4) | 1 | F | 412 | (400 MHz, DMSO-d₆) 1.19-1.32 (m, 1 H) 1.35-1.48 (m, 2 H) 1.65-1.77 (m, 3 H) 1.79-1.90 (m, 2 H) 2.39-2.48 (m, 2 H) 2.80-2.88 (m, 2 H) 3.40-3.47 (m, 2 H) 4.31-4.43 (m, 3 H) 6.99-7.14 (m, 4 H) 7.35 (s, 2 H) 7.85 (d, J = 3 Hz, 1 H) 8.05 (d, J = 3 Hz, 1 H) |
| 95 | 6-amino-N-(butan-2- yl)-5-cyclohexyl-5H- pyrrrolo[2,3- b]pyrazine-7- sulfonamide | 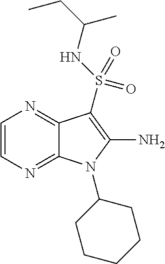 [SMILES: CCC(C)NS(=O)(=O)c1c(N)n(C2CCCCC2)c2nccnc12] | butan-2-amine (CAS 13952- 84-6) | 1 | E | 352 | (400 MHz, DMSO-d₆) 0.64 (t, J = 7 Hz, 3 H) 0.88 (d, J = 7 Hz, 3 H) 1.21-1.33 (m, 3 H) 1.36-1.49 (m, 2 H) 1.65-1.77 (m, 3 H) 1.80-1.92 (m, 2 H) 2.44-2.57 (m, 2 H) 3.08-3.19 (m, 1 H) 4.31-4.44 (m, 1 H) 7.02-7.11 (m, 1 H) 7.19 (s, 2 H) 7.88 (d, J = 3 Hz, 1 H) 8.08 (d, J = 3 Hz, 1 H) |
| 96 | 6-amino-5-cyclohexyl- N-(oxolan-2- ylmethyl)-5H- pyrrolo[2,3- b]pyrazine-7- sulfonamide | 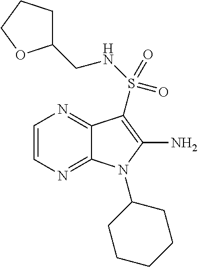 [SMILES: Nc1c(S(=O)(=O)NCC2CCCO2)c2nccnc2n1C1CCCCC1] | (tetrahydro- furan-2-yl)- methanamine (CAS 4795-29- 3) | 1 | E | 380 | (400 MHz, DMSO-d₆) 1.23-1.33 (m, 1 H) 1.36-1.55 (m, 3 H) 1.64-1.91 (m, 8 H) 2.42-2.54 (m, 2 H) 2.77-2.94 (m, 2 H) 3.46-3.56 (m, 1 H) 3.57-3.67 (m, 1 H) 3.73-3.86 (m, 1 H) 4.31-4.46 (m, 1 H) 7.07-7.15 (m, 1 H) 7.20 (s, 2 H) 7.89 (d, J = 3 Hz, 1 H) 8.08 (d, J = 3 Hz, 1 H) |
| 97 | 5-cyclohexyl-7-(2,3- dihydro-1H-isoindole- 2-sulfonyl)-5H- pyrrolo[2,3-b]pyrazin- 6-amine | 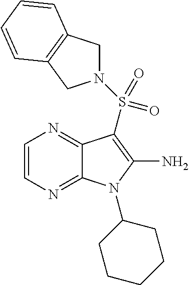 [SMILES: Nc1c(S(=O)(=O)N2Cc3ccccc3C2)c2nccnc2n1C1CCCCC1] | Isoindoline (CAS 496-12- 8) | 1 | F | 398 | (400 MHz, DMSO-d₆) 1.19-1.31 (m, 1 H) 1.34-1.47 (m, 2 H) 1.61-1.78 (m, 3 H) 1.78-1.89 (m, 2 H) 2.38-2.48 (m, 2 H) 4.30-4.44 (m, 1 H) 4.73 (s, 4 H) 7.13-7.26 (m, 4 H) 7.38 (s, 2 H) 7.82-7.85 (m, 1 H) 7.99-8.06 (m, 1 H) |
| 98 | 5-cyclohexyl-7-{4-[(4- fluorophenyl)carbonyl] piperazine-1-sulfonyl}- 5H-pyrrolo[2,3- b]pyrazin-6-amine | 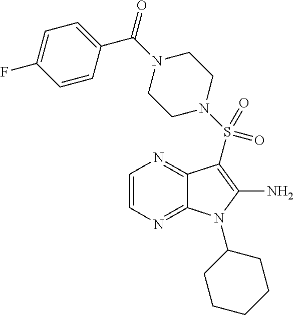 [SMILES: Nc1c(S(=O)(=O)N2CCN(C(=O)c3ccc(F)cc3)CC2)c2nccnc2n1C1CCCCC1] | (4- fluorophenyl) (piperazin-1- yl)methanone (CAS 102391- 98-0) | 1 | E | 487 | (400 MHz, DICHLOROMETHANE- d₂) 1.35-1.57 (m, 3 H) 1.78-1.87 (m, 1 H) 1.91-2.06 (m, 4 H) 2.39-2.53 (m, 2 H) 3.21-3.34 (m, 4 H) 3.55-3.85 (m, 4 H) 4.23-4.35 (m, 1 H) 5.95 (br. s., 2 H) 7.08-7.18 (m, 2 H) 7.33-740 (m, 2 H) 7.98 (d, J = 3 Hz, 1 H) 8.19 (d, J = 3 Hz, 1 H) |
| 99 | 5-cyclohexyl-7-(3- phenoxyazetidine-1- sulfonyl)-5H- pyrrolo[2,3-b]pyrazin- 6-amine | 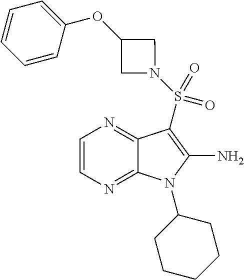 [SMILES: Nc1c(S(=O)(=O)N2CC(Oc3ccccc3)C2)c2nccnc2n1C1CCCCC1] | 3-phenoxy- azetidine (CAS 76263- 18-8 | 1 | F | 428 | (400 MHz, DICHLOROMETHANE- d₂) 1.35-1.56 (m, 3 H) 1.77-1.85 (m, 1 H) 1.92-2.06 (m, 4 H) 2.41-2.56 (m, 2 H) 4.06-4.16 (m, 2 H) 4.25-4.34 (m, 1 H) 4.36-4.41 (m, 2 H) 4.75-4.83 (m, 1 H) 6.00 (br. s., 2 H) 6.55-6.60 (m, 2 H) 6.93-7.01 (m, 1 H) 7.21-7.29 (m, 2 H) 7.99 (d, J = 3 Hz, 1 H) 8.18 (d, J = 3 Hz, 1 H) |
| 100 | 5-cyclohexyl-7-[3- (piperidin-1- yl)azetidine-1- sulfonyl]-5H- pyrrolo[2,3-b]pyrazin- 6-amine | 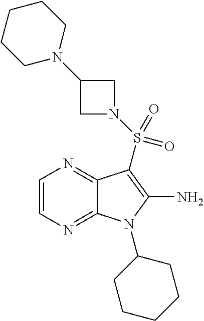 [SMILES: Nc1c(S(=O)(=O)N2CC(N3CCCCC3)C2)c2nccnc2n1C1CCCCC1] | 1-(azetidin-3- yl)piperidine (CAS 138022- 86-3) | 1 | E | 419 | (400 MHz, DMSO-d₆) 1.13-1.34 (m, 7 H) 1.37-1.52 (m, 2 H) 1.65-1.80 (m, 3 H) 1.83-1.97 (m, 6 H) 2.54-2.60 (m, 2 H) 2.84-2.95 (m, 1 H) 3.69-3.76 (m, 2 H) 3.76-3.83 (m, 2 H) 4.37-4.50 (m, 1 H) 7.32 (s, 2 H) 7.93 (d, J = 3 Hz, 1 H) 8.11 (d, J = 3 Hz, 1 H) |
| 101 | 5-cyclohexyl-7-[3-(1H- pyrazol-1-yl)azetidine- 1-sulfonyl]-5H- pyrrolo[2,3-b]pyrazin- 6-amine | 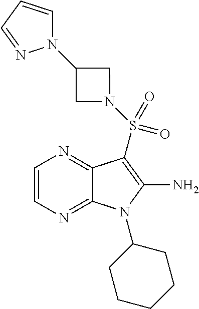 [SMILES: Nc1c(S(=O)(=O)N2CC(n3cccn3)C2)c2nccnc2n1C1CCCCC1] | 1-(azetidin-3- yl)-1H- pyrazole (CAS 1107627-16-6) | 1 | E | 402 | (400 MHz, DMSO-d₆) 1.24-1.35 (m, 1 H) 1.37-1.52 (m, 2 H) 1.65-1.94 (m, 5 H) 2.53-2.62 (m, 2 H) 4.22 (d, J = 7 Hz, 4 H) 4.38-4.51 (m, 1 H) 4.97-5.09 (m, 1 H) 6.06-6.14 (m, 1 H) 7.11-7.19 (m, 1 H) 7.35 (s, 2 H) 7.46-7.56 (m, 1 H) 7.91 (d, J = 3 Hz, 1 H) 8.05 (d, J = 3 Hz, 1 H) |
| 102 | 5-cyclohexyl-7-(3- methylpiperidine-1- sulfonyl)-5H- pyrrolo[2,3-b]pyrazin- 6-amine | 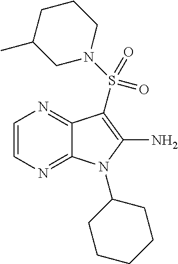 [SMILES: CC1CCCN(S(=O)(=O)c2c(N)n(C3CCCCC3)c3nccnc23)C1] | 3-methyl- piperidine (CAS 626-56- 2) | 1 | F | 378 | (400 MHz, DICHLOROMETHANE- d₂) 0.80-0.98 (m, 4 H) 1.33-1.67 (m, 4 H) 1.68-1.86 (m, 4 H) 1.90-2.05 (m, 4 H) 2.18-2.29 (m, 1 H) 2.40-2.54 (m, 2 H) 2.54-2.63 (m, 1 H) 3.64-3.77 (m, 2 H) 4.24-4.38 (m, 1 H) 6.00 (br. s., 2 H) 7.95 (d, J = 3 Hz, 1 H) 8.19 (d, J = 3 Hz, 1 H) |
| 103 | 6-amino-5-cyclohexyl- N-[2-(1,3-thiazol-2- yl)ethyl]-5H- pyrrolo[2,3- b]pyrazine-7- sulfonamide | 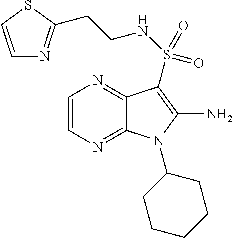 [SMILES: Nc1c(S(=O)(=O)NCCc2nccs2)c2nccnc2n1C1CCCCC1] | 2-(thiazol-2- yl)-ethanamine (CAS 18453- 07-1) | 1 | D | 407 | (400 MHz, DICHLOROMETHANE- d₂) 1.29-1.56 (m, 3 H) 1.76-1.85 (m, 1 H) 1.89-2.04 (m, 4 H) 2.38-2.54 (m, 2 H) 3.17-3.26 (m, 2 H) 3.34-3.46 (m, 2 H) 4.19-4.35 (m, 1 H) 5.82-6.05 (m, 3 H) 7.20 (d, J = 3 Hz, 1 H) 7.64 (d, J = 3 Hz, 1 H) 7.95 (d, J = 3 Hz, 1 H) 8.12 (d, J = 3 Hz, 1 H) |
| 104 | 8-{6-amino-5- cyclohexyl-5H- pyrrolo[2,3- b]pyrazine-7- sulfonyl}-8- azabicyclo[3.2.1]octan- 3-ol | 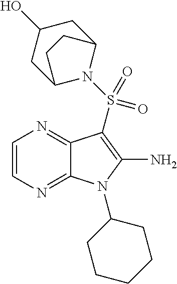 [SMILES: Nc1c(S(=O)(=O)N2C3CCC2CC(O)C3)c2nccnc2n1C1CCCCC1] | 8-azabicyclo- [3.2.1]octan-3- ol | 1 | D | 406 | (400 MHz, DICHLOROMETHANE- d₂) 1.33-1.58 (m, 6 H) 1.76-1.85 (m, 3 H) 1.86-1.94 (m, 2 H) 1.95-2.05 (m, 4 H) 2.17-2.25 (m, 2 H) 2.37-2.53 (m, 2 H) 4.05-4.15 (m, 1 H) 4.24-4.40 (m, 3 H) 5.96 (br. s., 2 H) 7.95 (d, J = 3 Hz, 1 H) 8.22 (d, J = 3 Hz, 1 H) |
| 105 | 5-cyclohexyl-7-[4- (2,2,2-trifluoroethyl)- piperazine-1-sulfonyl]- 5H-pyrrolo[2,3- b]pyrazin-6-amine | 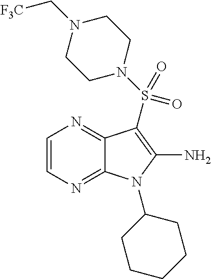 [SMILES: Nc1c(S(=O)(=O)N2CCN(CC(F)(F)F)CC2)c2nccnc2n1C1CCCCC1] | 1-(2,2,2- trifluoroethyl)- piperazine (CAS 13349- 90-1) | 1 | Reverse phase C18 5-95% water (+0.05% NH₃)/MeOH | 447 | (400 MHz, DICHLOROMETHANE- d₂) 1.34-1.55 (m, 3 H) 1.78-1.86 (m, 1 H) 1.89-2.05 (m, 4 H) 2.39-2.52 (m, 2 H) 2.73-2.83 (m, 4 H) 2.94-3.05 (m, 2 H) 3.19-3.31 (m, 4 H) 4.23-4.37 (m, 1 H) 5.94 (br. s., 2 H) 7.97 (d, J = 3 Hz, 1 H) 8.19 (d, J = 3 Hz, 1 H) |
| 106 | (1-{6-amino-5- cyclohexyl-5H- pyrrolo[2,3- b]pyrazine-7- sulfonyl}piperidin-4- yl)methanol | 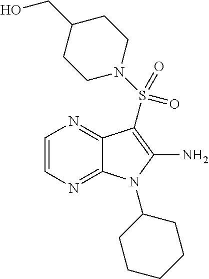 [SMILES: Nc1c(S(=O)(=O)N2CCC(CO)CC2)c2nccnc2n1C1CCCCC1] | piperidin-4-yl- methanol (CAS 6457-49- 4) | 1 | Reverse phase C18 5-95% water (+0.05% NH₃)/MeOH | 394 | (400 MHz, DICHLOROMETHANE- d₂) 1.31-1.52 (m, 6 H) 1.77-1.84 (m, 3 H) 1.90-2.04 (m, 4 H) 2.38-2.53 (m, 2 H) 2.56-2.66 (m, 3 H) 3.42-3.51 (m, 2 H) 3.83-3.91 (m, 2 H) 4.23-4.35 (m, 1 H) 5.92 (br. s., 2 H) 7.95 (d, J = 3 Hz, 1 H) 8.19 (d, J = 3 Hz, 1 H) |
| 107 | 5-cyclohexyl-7-[4- (cyclopropylmethoxy) piperidine-1-sulfonyl]- 5H-pyrrolo[2,3- b]pyrazin-6-amine | 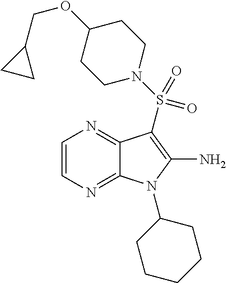 [SMILES: Nc1c(S(=O)(=O)N2CCC(OCC3CC3)CC2)c2nccnc2n1C1CCCCC1] | 4- (cyclopropyl- methoxy)- piperidine (CAS 865106- 51-0) | 1 | Reverse phase C18 5-95% water (+0.05% NH₃)/MeOH | 434 | (400 MHz, DICHLOROMETHANE- d₂) −0.05-0.07 (m, 2 H) 0.27-0.37 (m, 2 H) 0.77-0.90 (m, 1 H) 1.21-1.44 (m, 3 H) 1.50-1.59 (m, 2 H) 1.65-1.72 (m, 1 H) 1.74-1.90 (m, 6 H) 2.25-2.39 (m, 2 H) 2.87-2.94 (m, 2 H) 3.06-3.12 (m, 2 H) 3.20-3.29 (m, 1 H) 3.30-3.41 (m, 2 H) 4.12-4.25 (m, 1 H) 5.78-5.97 (m, 2 H) 7.84 (d, J = 3 Hz, 1 H) 8.06 (d, J = 3 Hz, 1 H) |

Examples 108 to 118 (see Table 4 following) were prepared using the general procedure 6 described below.

### Procedure 6

A solution of 2-(5-cyclohexyl-7-((4-methoxyphenyl)sulfonyl)-5H-pyrrolo[2,3-b]pyrazin-6-yl)isoindoline-1,3-dione (Intermediate 44; 56 mg, 0.108 mmol) and hydrazine monohydrate (11 μL, 0.22 mmol) in ethanol (1 mL) was stirred at 70° C. in a sealed tube for 1 h. The mixture was allowed to cool then (except where noted otherwise) diluted with DCM and filtered. The filtrate was concentrated in vacuo and the crude product was purified by column chromatography on silica with the indicated eluent, or by preparative reverse phase HPLC as indicated in the table to afford the title compound.

TABLE
Reverse phase preparative HPLC methods

| | Gradient (acetonitrile/water |
|---|---|
| Method | (with 0.1% ammonia unless indicated)) |
| A | 5-25% |
| B | 5-40% |
| C | 10-50% |
| D | 20-60% |
| E | 30-70% |
| F | 40-80% |
| G | 55-95% |
| H | 30-70% (0.1% formic acid) |

TABLE 4

| Ex- | | | | Puri- | | |
|---|---|---|---|---|---|---|
| am- | | | | fication | MS | ¹H NMR |
| ple | Compound Name | Structure | Starting material | Method | ES+ | data δ ppm |
| 108 | 5-cyclohexyl-7-[(4- methoxybenzene)- sulfonyl]-5H- pyrrolo[2,3-b]pyrazin- 6-amine | 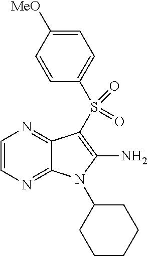 [SMILES: COc1ccc(S(=O)(=O)c2c(N)n(C3CCCCC3)c3nccnc23)cc1] | 2-(5-cyclohexyl-7-((4- methoxyphenyl)sulfonyl)- 5H-pyrrolo[2,3-b]pyrazin- 6-yl)isoindoline-1,3-dione (Intermediate 44) | 0-100% ethyl acetate/ petrol | 387 | (400 MHz, METHANOL-d₄) 1.32- 1.43 (m, 1 H) 1.45-1.57 (m, 2 H) 1.73- 1.83 (m, 3 H) 1.89-1.98 (m, 2 H) 2.52-2.64 (m, 2 H) 3.84 (s, 3 H) 4.27- 4.39 (m, 1 H) 6.99-7.06 (m, 2 H) 7.90 (d, J = 3 Hz, 1 H) 8.00-8.07 (m, 3 H) |
| 109 | 5-cyclohexyl-7- (cyclopropanesulfonyl)- 5H-pyrrolo[2,3- b]pyrazin-6-amine | 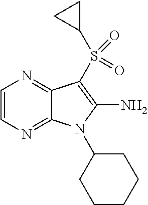 [SMILES: Nc1c(S(=O)(=O)C2CC2)c2nccnc2n1C1CCCCC1] | 2-(5-cyclohexyl-7- (cyclopropylsulfonyl)-5H- pyrrolo[2,3-b]pyrazin-6- yl)isoindoline-1,3-dione (Intermediate 45) | 0-100% ethyl acetate/ petrol | 321 | (400 MHz, DICHLOROMETHANE- d₂) 0.81-0.89 (m, 2 H) 1.20-1.46 (m, 5 H) 1.65-1.74 (m, 1 H) 1.78- 1.93 (m, 4 H) 2.25-2.43 (m, 2 H) 2.67-2.78 (m, 1 H) 4.09-4.23 (m, 1 H) 5.89 (br. s., 2 H) 7.82-7.89 (m, 1 H) 8.06-8.12 (m, 1 H) |
| 110 | 5-cyclohexyl-7-[(3- fluorobenzene)sulfonyl]- 5H-pyrrolo[2,3- b]pyrazin-6-amine | 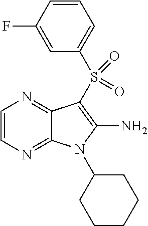 [SMILES: Nc1c(S(=O)(=O)c2cccc(F)c2)c2nccnc2n1C1CCCCC1] | 2-(5-cyclohexyl-7-((3- fluorophenyl)sulfonyl)-5H- pyrrolo[2,3-b]pyrazin-6- yl)isoindoline-1,3-dione (Intermediate 46) | 0-100% ethyl acetate/ petrol | 375 | (400 MHz, DICHLOROMETHANE- d₂) 1.17-1.41 (m, 3 H) 1.61-1.70 (m, 1 H) 1.71-1.89 (m, 4 H) 2.23-2.39 (m, 2 H) 4.05-4.21 (m, 1 H) 6.13 (br. s., 2 H) 7.09-7.19 (m, 1 H) 7.34- 7.42 (m, 1 H) 7.74-7.91 (m, 3 H) 8.06-8.13 (m, 1 H) |
| 111 | 5-cyclohexyl-7-[(2- fluorobenzene)sulfonyl]- 5H-pyrrolo[2,3- b]pyrazin-6-amine | 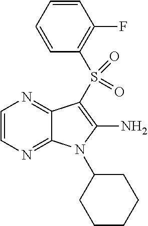 [SMILES: Nc1c(S(=O)(=O)c2ccccc2F)c2nccnc2n1C1CCCCC1] | 2-(5-cyclohexyl-7-((2- fluorophenyl)sulfonyl)-5H- pyrrolo[2,3-b]pyrazin-6- yl)isoindoline-1,3-dione (Intermediate 47) | 0-100% ethyl acetate/ petrol | 375 | (400 MHz, DICHLOROMETHANE- d₂) 1.16-1.47 (m, 3 H) 1.64-1.72 (m, 1 H) 1.76-1.94 (m, 4 H) 2.25-2.46 (m, 2 H) 4.09-4.26 (m, 1 H) 6.17 (br. s., 2 H) 6.95-7.05 (m, 1 H) 7.19- 7.30 (m, 1 H) 7.40-7.50 (m, 1 H) 7.76-7.83 (m, 1 H)7.94-8.01 (m, 1 H) 8.07-8.14 (m, 1 H) |
| 112 | 5-cyclohexyl-7-[(3- methoxybenzene)- sulfonyl]-5H- pyrrolo[2,3-b]pyrazin- 6-amine | 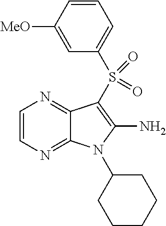 [SMILES: COc1cccc(S(=O)(=O)c2c(N)n(C3CCCCC3)c3nccnc23)c1] | 2-(5-cyclohexyl-7-((3- methoxyphenyl)sulfonyl)- 5H-pyrrolo[2,3-b]pyrazin- 6-yl)isoindoline-1,3-dione (Intermediate 48) | H | 387 | (400 MHz, DICHLOROMETHANE- d₂) 1.27-1.56 (m, 3 H) 1.75-1.81 (m, 1 H) 1.83-2.02 (m, 4 H) 2.35-2.49 (m, 2 H) 3.87 (s, 3 H) 4.20-4.31 (m, 1 H) 6.21 (br. s., 2 H) 7.05-7.12 (m, 1 H) 7.36-7.45 (m, 1 H) 7.69-7.73 (m, 1 H) 7.78-7.83 (m, 1 H) 7.95 (d, J = 3 Hz, 1 H) 8.22 (d, J = 3 Hz, 1 H) |
| 113 | 4-{6-amino-5- cyclohexyl-5H- pyrrolo[2,3-b]pyrazine- 7-sulfonyl}benzonitrile | 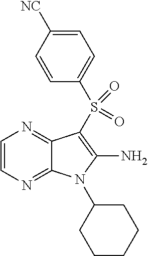 [SMILES: N#Cc1ccc(S(=O)(=O)c2c(N)n(C3CCCCC3)c3nccnc23)cc1] | 4-((5-cyclohexyl-6-(1,3- dioxoisoindolin-2-yl)-5H- pyrrolo[2,3-b]pyrazin-7- yl)sulfonyl)benzonitrile (Intermediate 49) | H | 382 | (400 MHz, DICHLOROMETHANE- d₂) 1.32-1.53 (m, 3 H) 1.76-1.83 (m, 1 H) 1.85-1.93 (m, 2 H) 1.94-2.05 (m, 2 H) 2.36-2.49 (m, 2 H) 4.18- 4.32 (m, 1 H) 6.20 (br. s., 2 H) 7.77- 7.84 (m, 2 H) 7.97 (d, J = 3 Hz, 1 H) 8.22 (d, J = 3 Hz, 1 H) 8.27-8.35 (m, 2 H) |
| 114 | 7-[(3-chloro-4- methoxybenzene)- sulfonyl]-5-cyclohexyl- 5H-pyrrolo[2,3- b]pyrazin-6-amine | 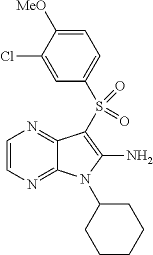 [SMILES: COc1ccc(S(=O)(=O)c2c(N)n(C3CCCCC3)c3nccnc23)cc1Cl] | 2-(7-((3-chloro-4- methoxyphenyl)sulfonyl)-5- cyclohexyl-5H-pyrrolo[2,3- b]pyrazin-6-yl)isoindoline- 1,3-dione (Intermediate 50) | Note 1 | 421 | (400 MHz, DICHLOROMETHANE- d₂) 1.32-1.56 (m, 3 H) 1.73-2.02 (m, 5 H) 2.34-2.51 (m, 2 H) 3.95 (s, 3 H) 4.19-4.35 (m, 1 H) 6.26 (br. s., 2 H) 7.04 (d, J = 9 Hz, 1 H) 7.95 (d, J = 3 Hz, 1 H) 8.09-8.17 (m, 2 H) 8.20 (d, J = 3 Hz, 1 H) |
| 115 | 5-cyclohexyl-7-(6- methoxypyridine-3- sulfonyl)-5H- pyrrolo[2,3-b]pyrazin- 6-amine | 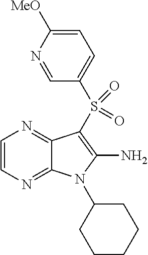 [SMILES: COc1ccc(S(=O)(=O)c2c(N)n(C3CCCCC3)c3nccnc23)cn1] | 2-(5-cyclohexyl-7-((6- methoxypyridin-3- yl)sulfonyl)-5H- pyrrolo[2,3-b]pyrazin-6- yl)isoindoline-1,3-dione (Intermediate 51) | 0-4% methanol/ DCM | 388 | (400 MHz, DICHLOROMETHANE- d₂) 1.28-1.55 (m, 3 H) 1.79 (d, J = 12 Hz, 1 H) 1.85-2.03 (m, 4 H) 2.36- 2.52 (m, 2 H) 3.58 (s, 3 H) 4.19-4.33 (m, 1 H) 6.18 (br. s., 2 H) 6.47 (d, J = 10 Hz, 1 H) 7.82-7.90 (m, 1 H) 7.96 (d, J = 3 Hz, 1 H) 8.19 (d, J = 3 Hz, 1 H) 8.41 (d, J = 3 Hz, 1 H) |
| 116 | 5-cyclohexyl-7-{[4- (trifluoromethoxy)- benzene]sulfonyl}-5H- pyrrolo[2,3-b]pyrazin- 6-amine | 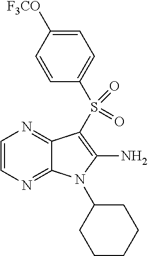 [SMILES: Nc1c(S(=O)(=O)c2ccc(OC(F)(F)F)cc2)c2nccnc2n1C1CCCCC1] | 2-(5-cyclohexyl-7-((4- (trifluoromethoxy)phenyl) sulfonyl)-5H-pyrrolo[2,3- b]pyrazin-6-yl)isoindoline- 1,3-dione (Intermediate 52) | F | 441 | (400 MHz, DICHLOROMETHANE- d₂) 1.25-1.51 (m, 3 H) 1.71-2.01 (m, 5 H) 2.30-2.48 (m, 2 H) 4.12-4.33 (m, 1 H) 6.15 (br. s., 2 H) 7.31 (d, J = 8 Hz, 2 H) 7.92 (d, J = 3 Hz, 1 H) 8.13- 8.28 (m, 3 H) |
| 117 | 5-cyclohexyl-7-(2,3- dihydro-1,4- benzodioxine-6- sulfonyl)-5H- pyrrolo[2,3-b]pyrazin- 6-amine | 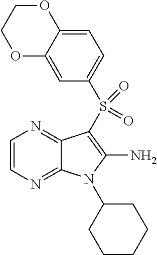 [SMILES: Nc1c(S(=O)(=O)c2ccc3c(c2)OCCO3)c2nccnc2n1C1CCCCC1] | 2-(5-cyclohexyl-7-((2,3- dihydrobenzo[b][1,4]dioxin- 6-yl)sulfonyl)-5H- pyrrolo[2,3-b]pyrazin-6- yl)isoindoline-1,3-dione (Intermediate 53) | E | 415 | (400 MHz, DICHLOROMETHANE- d₂) 1.24-1.51 (m, 3 H) 1.71-1.80 (m, 1 H) 1.81-1.99 (m, 4 H) 2.30-2.46 (m, 2 H) 4.15-4.30 (m, 5 H) 6.12 (br. s., 2 H) 6.91 (d, J = 8 Hz, 1 H) 7.58- 7.65 (m, 2 H) 7.90 (d, J = 3 Hz, 1 H) 8.17 (d, J = 3 Hz, 1 H) |
| 118 | 5-cyclohexyl-7-{[4- (difluoromethoxy)- benzene]sulfonyl}-5H- pyrrolo[2,3-b]pyrazin- 6-amine | 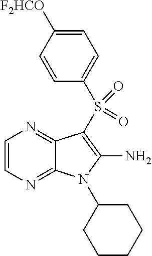 [SMILES: Nc1c(S(=O)(=O)c2ccc(OC(F)F)cc2)c2nccnc2n1C1CCCCC1] | 2-(5-cyclohexyl-7-((4- (difluoromethoxy)phenyl)- sulfonyl)-5H-pyrrolo[2,3- b]pyrazin-6-yl)isoindoline- 1,3-dione (Intermediate 54) | Note 2 | 443 | (400 MHz, DICHLOROMETHANE- d₂) 1.26-1.52 (m, 3 H) 1.71-2.00 (m, 5 H) 2.27-2.47 (m, 2 H) 4.12-4.32 (m, 1 H) 6.19 (br. s., 2 H) 6.59 (t, J = 74 Hz, 1 H) 7.19 (d, J = 9 Hz, 2 H) 7.92 (d, J = 3 Hz, 1 H) 8.14-8.23 (m, 3 H) |

Note 1
Alternative workup: The mixture was filtered and the precipitate was washed with ethanol. The filtrate was diluted with ether then concentrated in vacuo and the crude material was purified by column chromatography (silica, 10-55% EtOAc/petroleum ether gradient) to afford the title compound.
Note 2
Alternative workup: The reaction mixture was evaporated and the residue was dissolved in ethyl acetate then washed with dilute NaOH(aq), water, and saturated brine. The organic phase was dried (H-frit) and concentrated to afford the title compound.

### 3. Biological Efficacy of Compounds of the Invention

### Screening Protocol:

### Ca-Flux Functional Assay: Determination of Agonist/Positive Allosteric Modulator (PAM) Activity

GPR43 agonist/PAM activity was determined by measuring changes in intracellular calcium levels using a Ca²⁺ sensitive fluorescent dye. The changes in fluorescent signal were monitored by FLIPR (manufactured by Molecular Devices). GPR43 mediated increases in intracellular Ca²⁺ concentration were readily detected upon activation with sodium acetate. Prior to the assay (24 hours), CHO-K1 Gα16 cells stably expressing human GPR43 were-seeded in cell culture medium in black, clear-bottom 384-well plates (Corning Inc) and grown overnight at 37° C., 5% CO₂. On the day of the assay, cell culture media was removed and cells were loaded with Calcium 5 Dye (Molecular Devices) diluted in HBSS containing 25 mM HEPES, 2.5 mM Probenecid, 0.1% BSA for 1 hour at 37° C., 5% CO₂. 10 point half log concentration response curves of sodium acetate from 10 mM were conducted prior to the testing of compounds to calculate the sodium acetate concentration that produces 20% of the maximal response (EC₂₀). Test compounds (at to point half log concentration response curves from 10 μM) were added in the presence of sodium acetate to achieve a final concentration that produces approximately 20% maximal response as calculated from the previous experiment. The changes in fluorescent signal were monitored by FLIPR upon addition of the compound/EC₂₀ sodium acetate mix. The EC₅₀ values were determined from ten point concentration response curves. Curves were generated using the average of two wells for each data point.

The above assay detects both GPR43 receptor agonists and positive allosteric modulators of the GPR43 receptor, without distinguishing between the two. Activity in either regard is useful in the treatment of conditions associated with GPR43 receptor activity.

### Results:

| Compound | Mean | Compound | Mean | Compound | Mean |
|---|---|---|---|---|---|
| of Example | EC₅₀ | of Example | EC₅₀ | of Example | EC₅₀ |
| No. | (nM) | No. | (nM) | No. | (nM) |
| 1 | 261 | 2 | 218 | 3 | 122 |
| 4 | 908 | 5 | 195 | 6 | 301 |
| 7 | 172 | 8 | 118 | 9 | 1249 |
| 10 | 2298 | 11 | 753 | 12 | 671 |
| 13 | 129 | 14 | 138 | 15 | 381 |
| 16 | 337 | 17 | 559 | | |
| 19 | 1775 | 20 | 2359 | 21 | 282 |
| 22 | 167 | 23 | 1052 | 24 | 2586 |
| 25 | 1099 | 26 | 79 | 27 | 277 |
| 28 | 707 | 29 | 31 | 30 | 199 |
| 31 | 6936 | 32 | 689 | 33 | 2722 |
| 34 | 5985 | 35 | 1861 | 36 | 245 |
| 37 | 468 | 38 | 169 | 39 | 657 |
| 40 | 7377 | 41 | 585 | 42 | 2296 |
| 43 | 6985 | 44 | 1075 | 45 | 2892 |
| 46 | 5257 | 47 | 6486 | 48 | 3261 |
| 49 | 211 | 50 | 760 | 51 | 5998 |
| 52 | 822 | 53 | 275 | 54 | 1536 |
| 55 | 7169 | 56 | 1305 | 57 | 259 |
| 58 | 2235 | 59 | 792 | 60 | 1408 |
| 61 | 4316 | 62 | 303 | 63 | 7946 |
| 64 | 7131 | 65 | 210 | 66 | 327 |
| 67 | 830 | 68 | 1465 | 69 | 547 |
| 70 | 3276 | 71 | 8378 | 72 | 1795 |
| 73 | 488 | 74 | 321 | 75 | 1187 |
| 76 | 1528 | 77 | 290 | 78 | 337 |
| 79 | 5968 | 80 | 6066 | 81 | 1253 |
| 82 | 821 | 83 | 931 | 84 | 3288 |
| 85 | 1060 | 86 | 1715 | 87 | 1553 |
| 88 | 3145 | 89 | 658 | 90 | 6184 |
| 91 | 8352 | 92 | 440 | 93 | 5697 |
| 94 | 308 | 95 | 334 | 96 | 1955 |
| 97 | 173 | 98 | 2812 | 99 | 1551 |
| 100 | 3931 | 101 | 7031 | 102 | 258 |
| 103 | 471 | 104 | 3077 | 105 | 647 |
| 106 | 3052 | 107 | 320 | 108 | 78 |
| 109 | 2902 | 110 | 343 | 111 | 209 |
| 112 | 295 | 113 | 486 | 114 | 206 |
| 115 | 4564 | 116 | 1082 | 117 | 124 |
| 118 | 177 | | | | |

It will be understood that the present invention has been described above by way of example only. The examples are not intended to limit the scope of the present invention. Various modifications and embodiments can be made without departing from the scope and spirit of the invention, which is defined by the following claims.

1. A compound of formula (I): 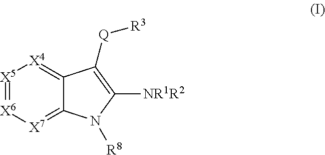
or a pharmaceutically acceptable salt thereof, wherein
Q represents —O—, —S—, —SO—, —SO₂—, —SO₂NR—, —SO₂(CH₂)ₘ— or —SO₂O—;
R represents a hydrogen atom or a C₁-C₆ alkyl group;
m is 1 or 2;
X⁴ represents N or CR⁴;
X⁵ represents N or CR⁵;
X⁶ represents N or CR⁶;
X⁷ represents N or CR⁷;
provided that one or two of X⁴, X⁵, X⁶ and X⁷ represents a nitrogen atom;
R¹ and R² each independently represent a hydrogen atom or a C₁-C₆ alkyl, C₃-C₈ cycloalkyl or C₁-C₆ alkoxycarbonyl group, each of which may be optionally substituted by at least one halogen atom;
R³ represents a saturated or unsaturated 3- to 10-membered ring system which may comprise at least one ring heteroatom independently selected from nitrogen, oxygen and sulphur, wherein the 3- to 10-membered ring system is optionally substituted by at least one substituent independently selected from halogen, hydroxyl, cyano, C₁-C₆ alkyl, C₁-C₆ haloalkyl, C₁-C₆ hydroxyalkyl, C₁-C₆ alkoxy, C₁-C₆ haloalkoxy, C₃-C₆ cycloalkylC₁-C₆ alkoxy, C₁-C₆ alkoxyC₁-C₆ alkyl, C₁-C₆ alkylC(O)NR¹⁴—, phenyl, (halo)phenylcarbonyl, phenoxy, benzyl, benzyloxycarbonyl and a saturated or unsaturated 4- to 6-membered heterocyclyl group, which heterocyclyl group is itself optionally substituted by at least one C₁-C₆ alkyl group,
and when Q represents —SO₂NR—, R³ may additionally represent a C₁-C₆ alkyl group optionally substituted by at least one substituent independently selected from halogen, C₁-C₆ alkoxy, C₃-C₆ cycloalkyl, phenyl and a saturated or unsaturated 4- to 6-membered heterocyclyl group;
R⁴, R⁵ and R⁶ each independently represent a hydrogen or a halogen atom, or a C₁-C₆ alkyl, C₁-C₆ alkoxy, C₁-C₆ alkylthio, C₁-C₆ haloalkyl, NR¹²R¹³, C₃-C₈ cycloalkyl or C₅-C₈ cycloalkenyl group;
R⁷ represents a hydrogen or a halogen atom, hydroxyl, cyano, NR⁹R¹⁰, or a C₁-C₆ alkyl, C₃-C₈ cycloalkyl, C₂-C₆ alkenyl, C₅-C₈ cycloalkenyl, C₁-C₆ alkoxy, C₃-C₈ cycloalkyloxy, benzyloxy, 3-to 11-membered saturated heterocyclyl, 3-to 11-membered saturated heterocyclyloxy, C₆-C₁₀ aryl or heteroaryl group, each of which may be optionally substituted by at least one substituent independently selected from halogen, cyano, C₁-C₆ alkyl, C₁-C₆ alkoxy, C₃-C₈ cycloalkyl, phenyl and a saturated or unsaturated 4- to 6-membered heterocyclyl group wherein each C₁-C₆ alkyl, C₁-C₆ alkoxy, C₃-C₈ cycloalkyl, phenyl or saturated or unsaturated 4- to 6-membered heterocyclyl substituent group may itself be optionally substituted by at least one substituent independently selected from halogen, C₁-C₃ alkyl, C₁-C₃ alkoxy and C₃-C₆ cycloalkyl;
either R⁸ represents a saturated 3- to 8-membered ring system which may comprise at least one ring heteroatom independently selected from nitrogen, oxygen and sulphur, wherein the 3- to 8-membered ring system is optionally substituted by at least one substituent independently selected from halogen, hydroxyl and C₁-C₆ alkyl, or R⁸ represents a C₁-C₆ alkyl group optionally substituted by at least one substituent independently selected from phenyl and C₃-C₆ cycloalkyl, the cycloalkyl group itself being optionally substituted by at least one C₁-C₆ alkyl group;
R⁹ and R¹⁰ each independently represent a hydrogen atom, or a C₁-C₆ alkyl or —(CH2)ₚ—R¹¹ group, each of which may be optionally substituted by at least one substituent independently selected from halogen, C₁-C₃ alkyl and C₁-C₃ alkoxy;
p is 0 or 1;
R¹¹ represents C₃-C₆ cycloalkyl, phenyl or a saturated or unsaturated 5- to 6-membered heterocyclyl group; and
R¹², R¹³ and R¹⁴ each independently represent a hydrogen atom or a C₁-C₆ alkyl group.

2. A compound according to claim 1, wherein X⁴ and X⁷ are N, X⁵ is CR⁵ and X⁶ is CR⁶.

3. A compound according to claim 1, wherein X⁴ and X⁶ are both N, X⁵ is CR⁵ and X⁷ is CR⁷.

4. A compound according to claim 1, wherein Q represents —SO₂—.

5. A compound according to claim 1, wherein R¹ and R² are both hydrogen.

6. A compound according to claim 1, wherein R³ represents a saturated or unsaturated 3- to 10-membered ring system which may comprise at least one ring heteroatom independently selected from nitrogen, oxygen and sulphur, optionally substituted as defined in claim 1, wherein the ring system is selected from phenyl, thienyl, cyclopropyl, cyclohexyl, pyridinyl, pyrrolidinyl, piperidinyl, piperazinyl, morpholinyl, azetidinyl, 1,4-oxazepanyl, azepanyl, thiomorpholinyl, 1,2,3,4-tetrahydroisoquinolinyl, 2,3-dihydroisoindolyl, azabicyclo[3.2.1]octanyl and 2,3-dihydro-1,4-benzodioxinyl.

7. A compound according to claim 1, wherein R³ represents phenyl optionally substituted by one or two substituents independently selected from fluorine, chlorine, cyano, methyl, trifluoromethyl, difluoromethoxy, trifluoromethoxy and C₁-C₃ alkoxy.

8. A compound according to claim 1, wherein R⁸ represents a saturated 4- to 7-membered ring system which may comprise at least one ring heteroatom independently selected from nitrogen, oxygen and sulphur, wherein the 4- to 7-membered ring system is optionally substituted by at least one substituent independently selected from halogen, hydroxyl and C₁-C₂ alkyl, or R⁸ represents a C₁-C₂ alkyl group optionally substituted by at least one substituent independently selected from phenyl and C₃-C₆ cycloalkyl, the cycloalkyl group itself being optionally substituted by at least one C₁-C₂ alkyl group.

9. A compound according to claim 1, wherein R⁸ represents a C₄-C₆ cycloalkyl group optionally substituted by at least one substituent independently selected from fluorine, hydroxyl and methyl.

10. A compound according to claim 1, selected from the group consisting of:
7-(benzenesulfonyl)-5-cyclohexyl-5H-pyrrolo[2,3-b]pyrazin-6-amine,
5-cycloheptyl-7-[(4-methylbenzene)sulfonyl]-5H-pyrrolo[2,3-b]pyrazin-6-amine,
5-cycloheptyl-7-[(4-methylbenzene)sulfonyl]-5H-pyrrolo[2,3-b]pyrazin-6-amine,
5-cyclopentyl-7-[(4-methylbenzene)sulfonyl]-5H-pyrrolo[2,3-b]pyrazin-6-amine,
7-[(4-chlorobenzene)sulfonyl]-5-cyclohexyl-5H-pyrrolo[2,3-b]pyrazin-6-amine,
5-cyclohexyl-7-[(4-fluorobenzene)sulfonyl]-5H-pyrrolo[2,3-b]pyrazin-6-amine,
5-cyclohexyl-7-{[4-(propan-2-yloxy)benzene]sulfonyl}-5H-pyrrolo[2,3-b]pyrazin-6-amine,
5-cyclohexyl-7-(thiophene-2-sulfonyl)-5H-pyrrolo[2,3-b]pyrazin-6-amine,
3-(benzenesulfonyl)-1-cyclohexyl-1H-pyrrolo[3,2-b]pyridin-2-amine,
1-cyclopentyl-3-[(4-methylbenzene)sulfonyl]-1H-pyrrolo[3,2-b]pyridin-2-amine,
1-cyclohexyl-3-[(4-methylbenzene)sulfonyl]-1H-pyrrolo[2,3-b]pyridin-2-amine,
7-(cyclohexanesulfonyl)-5-cyclohexyl-5H-pyrrolo[2,3-b]pyrazin-6-amine,
5-(4,4-difluorocyclohexyl)-7-[(4-methoxybenzene)sulfonyl]-5H-pyrrolo[2,3-b]pyrazin-6-amine,
1-(4,4-difluorocyclohexyl)-3-[(4-methoxybenzene)sulfonyl]-1H-pyrrolo[2,3-b]pyridin-2-amine,
3-(benzenesulfonyl)-1-cyclohexyl-1H-pyrrolo[2,3-b]pyridin-2-amine,
3-(benzenesulfonyl)-1-cyclohexyl-1H-pyrrolo[2,3-b]pyridin-2-amine,
3-(benzenesulfonyl)-1-(4,4-difluorocyclohexyl)-1H-pyrrolo[2,3-b]pyridin-2-amine,
7-(benzenesulfonyl)-5-cyclohexyl-5H-pyrrolo[3,2-d]pyrimidin-6-amine,
3-(benzenesulfonyl)-1-cyclohexyl-1H-pyrrolo[2,3-c]pyridin-2-amine,
3-(benzenesulfonyl)-1-(4,4-difluorocyclohexyl)-1H-pyrrolo[3,2-b]pyridin-2-amine,
1-(4,4-difluorocyclohexyl)-3-[(4-methoxybenzene)sulfonyl]-1H-pyrrolo[3,2-b]pyridin-2-amine,
3-(benzenesulfonyl)-1-cyclohexyl-1H-pyrrolo[3,2-c]pyridin-2-amine,
methyl N-[7-(benzenesulfonyl)-5-cyclohexyl-5H-pyrrolo[2,3-b]pyrazin-6-yl]carbamate,
3-(benzenesulfonyl)-1-(4,4-difluorocyclohexyl)-6-methyl-1H-pyrrolo[2,3-b]pyridin-2-amine,
7-(benzenesulfonyl)-5-cyclohexyl-4-methoxy-5H-pyrrolo[3,2-d]pyrimidin-6-amine,
5-(benzenesulfonyl)-3-chloro-7-cyclohexyl-7H-pyrrolo[2,3-c]pyridazin-6-amine,
5-(benzenesulfonyl)-7-cyclohexyl-7H-pyrrolo[2,3-c]pyridazin-6-amine,
7-(benzenesulfonyl)-5-(4,4-difluorocyclohexyl)-4-ethoxy-5H-pyrrolo[3,2-d]pyrimidin-6-amine,
7-(benzenesulfonyl)-4-(benzyloxy)-5-(4,4-difluorocyclohexyl)-5H-pyrrolo[3,2-d]pyrimidin-6-amine,
6-amino-5-(4,4-difluorocyclohexyl)-7-(phenylsulfonyl)-5H-pyrrolo[3,2-d]pyrimidin-4-ol,
7-(benzenesulfonyl)-4-chloro-5-(4,4-difluorocyclohexyl)-5H-pyrrolo[3,2-d]pyrimidin-6-amine,
7-(benzenesulfonyl)-5-(4,4-difluorocyclohexyl)-4-N-methyl-5H-pyrrolo[3,2-d]pyrimidine-4,6-diamine,
7-(benzenesulfonyl)-5-cyclohexyl-4-N,4-N-dimethyl-5H-pyrrolo[3,2-d]pyrimidine-4,6-diamine,
7-(benzenesulfonyl)-5-cyclopentyl-4-methoxy-5H-pyrrolo[3,2-d]pyrimidin-6-amine,
3-(benzenesulfonyl)-1-cyclohexyl-7-methoxy-1H-pyrrolo[2,3-c]pyridin-2-amine,
6-amino-7-(benzenesulfonyl)-5-cyclohexyl-5H-pyrrolo[3,2-d]pyrimidine-carbonitrile,
5-cyclohexyl-7-(2-fluorobenzenesulfonyl)-4-methoxy-2-methyl-5H-pyrrolo[3,2-d]pyrimidin-6-amine,
5-cyclohexyl-7-(3-fluorobenzenesulfonyl)-4-methoxy-2-methyl-5H-pyrrolo[3,2-d]pyrimidin-6-amine,
7-(benzenesulfonyl)-4-methoxy-5-(oxan-4-yl)-5H-pyrrolo[3,2-d]pyrimidin-6-amine,
6-amino-5-cyclohexyl-N-phenyl-5H-pyrrolo[2,3-b]pyrazine-7-sulfonamide,
6-amino-5-cyclohexyl-N-(pyridin-3-yl)-5H-pyrrolo[2,3-b]pyrazine-7-sulfonamide,
5-cyclobutyl-7-(phenylsulfonyl)-5H-pyrrolo[2,3-b]pyrazin-6-amine,
5-(2-methylcyclohexyl)-7-(phenylsulfonyl)-5H-pyrrolo[2,3-b]pyrazin-6-amine,
5-butyl-7-(phenylsulfonyl)-5H-pyrrolo[2,3-b]pyrazin-6-amine,
5-phenethyl-7-(phenylsulfonyl)-5H-pyrrolo[2,3-b]pyrazin-6-amine,
2-(6-amino-7-(phenylsulfonyl)-5H-pyrrolo[2,3-b]pyrazin-5-yl)cyclohexanol,
5-(2-cyclopropylethyl)-7-(phenylsulfonyl)-5H-pyrrolo[2,3-b]pyrazin-6-amine,
5-(4,4-difluoro-cyclohexyl)-7-(phenylsulfonyl)-5H-pyrrolo[2,3-b]pyrazin-6-amine,
5-(2-cyclobutylethyl)-7-(phenylsulfonyl)-5H-pyrrolo[2,3-b]pyrazin-6-amine,
7-(phenylsulfonyl)-5-(tetrahydro-2H-pyran-3-yl)-5H-pyrrolo[2,3-b]pyrazin-6-amine,
5-(3,3-dimethylbutyl)-7-(phenylsulfonyl)-5H-pyrrolo[2,3-b]pyrazin-6-amine,
5-((1R*,2R*,4S*)-bicyclo[2.2.1]heptan-2-yl)-7-(phenylsulfonyl)-5H-pyrrolo[2,3-b]pyrazin-6-amine,
5-(cyclopentylmethyl)-7-(phenylsulfonyl)-5H-pyrrolo[2,3-b]pyrazin-6-amine,
5-((1-ethyl cyclopropyl)-methyl)-7-(phenylsulfonyl)-5H-pyrrolo[2,3-b]pyrazin-6-amine,
5-((2,2-dimethylcyclopropyl)methyl)-7-(phenylsulfonyl)-5H-pyrrolo[2,3-b]pyrazin-6-amine,
5-cyclohexyl-7-(piperidin-1-ylsulfonyl)-5H-pyrrolo[2,3-b]pyrazin-6-amine,
5-cyclohexyl-7-(pyrrolidin-1-ylsulfonyl)-5H-pyrrolo[2,3-b]pyrazin-6-amine,
6-amino-5-cyclohexyl-N-propyl-5H-pyrrolo[2,3-b]pyrazine-7-sulfonamide,
6-amino-5-cyclohexyl-N-methyl-N-propyl-5H-pyrrolo[2,3-b]pyrazine-7-sulfonamide,
5-cyclohexyl-7-(morpholinosulfonyl)-5H-pyrrolo[2,3-b]pyrazin-6-amine,
5-cyclohexyl-7-((4-methylpiperidin-1-yl)sulfonyl)-5H-pyrrolo[2,3-b]pyrazin-6-amine,
5-cyclohexyl-7-((4-methylpiperazin-1-yl)sulfonyl)-5H-pyrrolo[2,3-b]pyrazin-6-amine,
5-cyclohexyl-7-((3-methoxyazetidin-1-yl)sulfonyl)-5H-pyrrolo[2,3-b]pyrazin-6-amine,
5-cyclohexyl-7-((4-ethoxypiperidin-1-yl)sulfonyl)-5H-pyrrolo[2,3-b]pyrazin-6-amine,
5-cyclohexyl-7-((4,4-dimethylpiperidin-1-yl)sulfonyl)-5H-pyrrolo[2,3-b]pyrazin-6-amine,
5-cyclohexyl-7-((3-methylpyrrolidin-1-yl)sulfonyl)-5H-pyrrolo[2,3-b]pyrazin-6-amine,
5-cyclohexyl-7-((2-methylpyrrolidin-1-yl)sulfonyl)-5H-pyrrolo[2,3-b]pyrazin-6-amine,
5-cyclohexyl-7-((4,4-difluoropiperidin-1-yl)sulfonyl)-5H-pyrrolo[2,3-b]pyrazin-6-amine,
6-amino-N-benzyl-5-cyclohexyl-5H-pyrrolo[2,3-b]pyrazine-7-sulfonamide,
6-amino-N,5-dicyclohexyl-N-methyl-5H-pyrrolo[2,3-b]pyrazine-7-sulfonamide,
5-cyclohexyl-7-(1,4-oxazepane-4-sulfonyl)-5H-pyrrolo[2,3-b]pyrazin-6-amine,
5-cyclohexyl-7-(4-methoxypiperidine-1-sulfonyl)-5H-pyrrolo[2,3-b]pyrazin-6-amine,
6-amino-N-(cyclobutylmethyl)-5-cyclohexyl-5H-pyrrolo[2,3-b]pyrazine-7-sulfonamide,
5-cyclohexyl-7-(3,3-dimethylpyrrolidine-1-sulfonyl)-5H-pyrrolo[2,3-b]pyrazin-6-amine,
5-cyclohexyl-7-(2,6-dimethylmorpholine-4-sulfonyl)-5H-pyrrolo[2,3-b]pyrazin-6-amine,
7-(azepane-1-sulfonyl)-5-cyclohexyl-5H-pyrrolo[2,3-b]pyrazin-6-amine,
5-cyclohexyl-7-(thiomorpholine-4-sulfonyl)-5H-pyrrolo[2,3-b]pyrazin-6-amine,
N-(1-{6-amino-5-cyclohexyl-5H-pyrrolo[2,3-b]pyrazine-7-sulfonyl}piperidin-4-yl)-N-methylacetamide,
6-amino-5-cyclohexyl-N-(oxetan-3-ylmethyl)-5H-pyrrolo[2,3-b]pyrazine-7-sulfonamide,
7-(4-benzylpiperidine-1-sulfonyl)-5-cyclohexyl-5H-pyrrolo[2,3-b]pyrazin-6-amine,
6-amino-5-cyclohexyl-N-(3,3,3-trifluoropropyl)-5H-pyrrolo[2,3-b]pyrazine-7-sulfonamide,
5-cyclohexyl-7-(4-phenylpiperidine-1-sulfonyl)-5H-pyrrolo[2,3-b]pyrazin-6-amine,
6-amino-5-cyclohexyl-N-(2-phenylethyl)-5H-pyrrolo[2,3-b]pyrazine-7-sulfonamide,
5-cyclohexyl-7-(4-phenoxypiperidine-1-sulfonyl)-5H-pyrrolo[2,3-b]pyrazin-6-amine,
5-cyclohexyl-7-(3-phenylpyrrolidine-1-sulfonyl)-5H-pyrrolo[2,3-b]pyrazin-6-amine,
5-cyclohexyl-7-[4-(trifluoromethyl)piperidine-1-sulfonyl]-5H-pyrrolo[2,3-b]pyrazin-6-amine,
5-cyclohexyl-7-[3-(methoxymethyl)pyrrolidine-1-sulfonyl]-5H-pyrrolo[2,3-b]pyrazin-6-amine,
6-amino-5-cyclohexyl-N-(cyclopropylmethyl)-5H-pyrrolo[2,3-b]pyrazine-7-sulfonamide,
6-amino-5-cyclohexyl-N-(2-methoxyethyl)-5H-pyrrolo[2,3-b]pyrazine-7-sulfonamide,
5-cyclohexyl-7-(3-methoxypyrrolidine-1-sulfonyl)-5H-pyrrolo[2,3-b]pyrazin-6-amine,
5-cyclohexyl-7-(3,3-dimethylpiperidine-1-sulfonyl)-5H-pyrrolo[2,3-b]pyrazin-6-amine,
1-{6-amino-5-cyclohexyl-5H-pyrrolo[2,3-b]pyrazine-7-sulfonyl}piperidin-4-ol,
5-cyclohexyl-7-(1,2,3,4-tetrahydroisoquinoline-2-sulfonyl)-5H-pyrrolo[2,3-b]pyrazin-6-amine,
6-amino-N-(butan-2-yl)-5-cyclohexyl-5H-pyrrolo[2,3-b]pyrazine-7-sulfonamide,
6-amino-5-cyclohexyl-N-(oxolan-2-ylmethyl)-5H-pyrrolo[2,3-b]pyrazine-7-sulfonamide,
5-cyclohexyl-7-(2,3-dihydro-1H-isoindole-2-sulfonyl)-5H-pyrrolo[2,3-b]pyrazin-6-amine,
5-cyclohexyl-7-{4-[(4-fluorophenyl)carbonyl]piperazine-1-sulfonyl}-5H-pyrrolo[2,3-b]pyrazin-6-amine,
5-cyclohexyl-7-(3-phenoxyazetidine-1-sulfonyl)-5H-pyrrolo[2,3-b]pyrazin-6-amine,
5-cyclohexyl-7-[3-(piperidin-1-yl)azetidine-1-sulfonyl]-5H-pyrrolo[2,3-b]pyrazin-6-amine,
5-cyclohexyl-7-[3-(1H-pyrazol-1-yl)azetidine-1-sulfonyl]-5H-pyrrolo[2,3-b]pyrazin-6-amine,
5-cyclohexyl-7-(3-methylpiperidine-1-sulfonyl)-5H-pyrrolo[2,3-b]pyrazin-6-amine,
6-amino-5-cyclohexyl-N-[2-(1,3-thiazol-2-yl)ethyl]-5H-pyrrolo[2,3-b]pyrazine-7-sulfonamide,
8-{6-amino-5-cyclohexyl-5H-pyrrolo[2,3-b]pyrazine-7-sulfonyl}-8-azabicyclo[3.2.1]octan-3-ol,
5-cyclohexyl-7-[4-(2,2,2-trifluoroethyl)-piperazine-1-sulfonyl]-5H-pyrrolo[2,3-b]pyrazin-6-amine,
(1-{6-amino-5-cyclohexyl-5H-pyrrolo[2,3-b]pyrazine-7-sulfonyl}piperidin-4-yl)methanol,
5-cyclohexyl-7-[4-(cyclopropylmethoxy)piperidine-1-sulfonyl]-5H-pyrrolo[2,3-b]pyrazin-6-amine,
5-cyclohexyl-7-[(4-methoxybenzene)-sulfonyl]-5H-pyrrolo[2,3-b]pyrazin-6-amine,
5-cyclohexyl-7-(cyclopropanesulfonyl)-5H-pyrrolo[2,3-b]pyrazin-6-amine,
5-cyclohexyl-7-[(3-fluorobenzene)sulfonyl]-5H-pyrrolo[2,3-b]pyrazin-6-amine,
5-cyclohexyl-7-[(2-fluorobenzene)sulfonyl]-5H-pyrrolo[2,3-b]pyrazin-6-amine,
5-cyclohexyl-7-[(3-methoxybenzene)-sulfonyl]-5H-pyrrolo[2,3-b]pyrazin-6-amine,
4-{6-amino-5-cyclohexyl-5H-pyrrolo[2,3-b]pyrazine-7-sulfonyl}benzonitrile,
7-[(3-chloro-4-methoxybenzene)-sulfonyl]-5-cyclohexyl-5H-pyrrolo[2,3-b]pyrazin-6-amine,
5-cyclohexyl-7-(6-methoxypyridine-3-sulfonyl)-5H-pyrrolo[2,3-b]pyrazin-6-amine,
5-cyclohexyl-7-{[4-(trifluoromethoxy)-benzene]sulfonyl}-5H-pyrrolo[2,3-b]pyrazin-6-amine,
5-cyclohexyl-7-(2,3-dihydro-1,4-benzodioxine-6-sulfonyl)-5H-pyrrolo[2,3-b]pyrazin-6-amine,
5-cyclohexyl-7-{[4-(difluoromethoxy)-benzene]sulfonyl}-5H-pyrrolo[2,3-b]pyrazin-6-amine,
and pharmaceutically acceptable salts of any one thereof.

11. A process for the preparation of a compound of formula (I) or a pharmaceutically acceptable salt thereof, as defined in claim 1 which comprises
(a) when NR¹R² represents NH₂, reacting a compound of formula 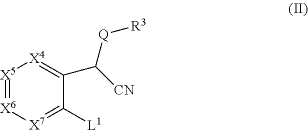
wherein L¹ represents a leaving group and X⁴, X⁵, X⁶, X⁷, Q and R³ are as defined in formula (I), with a compound of formula (III), H₂NR⁸, or a salt thereof wherein R⁸ is as defined in formula (I); or
(b) when NR¹R² represents NH₂, reacting a compound of formula 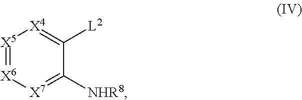
wherein L² represents a leaving group and X⁴, X⁵, X⁶, X⁷ and R⁸ are as defined in formula (I), with a compound of formula 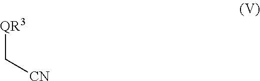
wherein Q and R³ are as defined in formula (I);
wherein any of compounds (II), (III), (IV) or (V) may optionally be protected;
and optionally thereafter carrying out one or more of the following procedures:
removing any protecting groups
converting a compound of formula (I) into another compound of formula (I)
forming a pharmaceutically acceptable salt.

12. A pharmaceutical composition comprising a compound of formula (I) or a pharmaceutically acceptable salt thereof, as claimed in claim 1, in association with a pharmaceutically acceptable adjuvant, diluent or carrier, and optionally one or more other therapeutic agents.

13. (canceled)

14. A method for treating a condition whose development or symptoms are linked to GPR43 receptor activity, comprising administering a compound of formula (I) as claimed in claim 1 or a pharmaceutically acceptable salt thereof.

15. A method for treating obesity and/or diabetes, comprising administering a compound of formula (I) as claimed in claim 1 or a pharmaceutically acceptable salt thereof.

16. A method for treating inflammatory bowel disease, comprising administering a compound of formula (I) as claimed in claim 1 or a pharmaceutically acceptable salt thereof.
